(19) United States
(12) Patent Application Publication
(10) Pub. No.: US 2024/0355465 A1
(43) Pub. Date: Oct. 24, 2024

(54) Information Management and Inventory Tracking for Medical Devices

(71) Applicant: PSIP2 LLC, Manchester, NH (US)

(72) Inventors: Stephen AMOS, Portsmouth, NH (US); Colleen A. Cassidy, Nolensville, TN (US); John M. Cronk, Strafford, NH (US); Larry Foster, Memphis, TN (US); Justin Y. Selleck, Victor, NY (US); Peter J. Tarsney, Northbrook, IL (US); Blaine Warkentine, Boulder, CO (US)

(73) Assignee: PSIP2 LLC, Manchester, NH (US)

(21) Appl. No.: 18/403,439

(22) Filed: Jan. 3, 2024

Related U.S. Application Data
(63) Continuation-in-part of application No. 18/370,375, filed on Sep. 19, 2023.
(60) Provisional application No. 63/538,485, filed on Sep. 14, 2023.
(60) Provisional application No. 63/534,855, filed on Aug. 27, 2023.
(60) Provisional application No. 63/531,239, filed on Aug. 7, 2023.
(60) Provisional application No. 63/437,115, filed on Jan. 4, 2023.

(51) Int. Cl. G16H 40/40

(52) U.S. Cl. CPC G16H 40/40

(57) ABSTRACT

During manufacture of a medical device, parameters if the device are measured, and results of that measurement are stored in a database. For example, parameters of an endoscope's image sensor may be measured and stored in the database. The medical device has a machine-readable serial number. A computer reads the serial number and records a reduction of an on-hand inventory of the medical devices. When the on-hand inventory warrants reordering to replenish the on-hand inventory. When warranted, the computer places an order with a supplier of medical devices of the class to replenish inventory. A computer controlling the medical device may read the database to obtain the measured properties. The obtained data may be used to calibrate or adjust the device's function. For example, white balance and color correction of an endoscope may be used to compute normalized video.

This application claims benefit as a continuation-in-part (C-I-P) of U.S. application Ser. No. 18/370,375, filed Sep. 19, 2023, titled Image Enhancement for Endoscope; claims benefit of U.S. Provisional application Ser. No. 63/538,485, filed Sep. 14, 2023, titled Endoscope; claims benefit of U.S. Provisional application Ser. No. 63/534,855, filed Aug. 27, 2023, titled Endoscope; claims benefit of U.S. Provisional application Ser. No. 63/531,239, filed Aug. 7, 2023, titled Endoscope; claims benefit of U.S. Provisional application Ser. No. 63/437,115, filed Jan. 4, 2023, titled Endoscope with Identification and Configuration Information. The benefit applications are incorporated by reference.

### BACKGROUND

This application relates to endoscopes, laparoscopes, arthroscopes, colonoscopes, and similar surgical devices or appliances specially adapted or intended to be used for evaluating, examining, measuring, monitoring, studying, or testing living or dead human and animal bodies for medical purposes, or for use in operative surgery upon the body or in preparation for operative surgery, together with devices designed to assist in operative surgery.

An endoscope may be an arthroscope (for joint surgery), a laparoscope (for abdominal surgery), colonoscope (rectum, colon, and lower small intestine), cystoscope (bladder and urethra), encephaloscope (brain), hysteroscope (vagina, cervix, uterus, and fallopian tubes), sinuscope (ear, nose, throat), thoracoscope (chest outside the lungs), tracheoscope (trachea and bronchi), esophageoscope (esophagus and stomach), etc. An endoscope may have a rigid shaft or a flexible insertion tube.

### SUMMARY

In general, in a first aspect, the invention features a method. A computer reads a machine-readable serial number of a medical device as the medical device is put into use. Based on the reading of the serial number, a computer records a reduction of an on-hand inventory level of a class of medical devices of which the medical device is a member. A computer computes when the reduction of on-hand inventory of the class indicates a level that warrants reordering to replenish the on-hand inventory. When warranted, a computer places an order with a supplier of medical devices of the class to replenish on-hand inventory of medical devices of the class.

In general, in a second aspect, the invention features a computer system. The system has one or more processors and a machine readable, non-transitory memory having stored therein instructions. The instructions are programmed to cause the processor(s) to read a machine-readable serial number of a medical device as the medical device is put into use, and based on the reading of the serial number, to record a reduction of an on-hand inventory level of a class of medical devices of which the medical device is a member. The instructions are programmed to cause the processor(s) to compute when the reduction of on-hand inventory of the class indicates a level that warrants reordering to replenish the on-hand inventory. The instructions are programmed to cause the processor(s) to place an order with a supplier of medical devices of the class to replenish on-hand inventory of medical devices of the class.

In general, in a third aspect, the invention features an endoscope. The endoscope has an image sensor designed to gather photons to produce video. The image sensor is of a type that has properties that vary from one sensor to another within manufacturing tolerances. The endoscope has a connector to connect the endoscope to a computer image processor. The image processor is programmed to obtain data from a database designed to store properties of the endoscope's image sensor and/or the image sensor's behavior, the database storing properties specific to specific sensors or a specific class of sensors. The obtained data describes the image sensor's properties from the database. The image processor is programmed to compute normalized video based on the obtained properties and the video from the image sensor.

In general, in a fourth aspect, the invention features a method. A computer processor programmed to receive image date from an image sensor of an endoscope, obtains data from a database designed to store properties of endoscopes' image sensors and/or the image sensors' behavior. The image sensor is of a type that has properties that vary from one sensor to another, the database storing properties specific to specific sensors or a specific class of sensors. A computer computes normalized video based on the obtained properties and the video from the image sensor.

In general, in a fifth aspect, the invention features a method. A database stores information relating to specific individual medical devices, including physical location and/or ownership. An optical reader reads 2D identification codes on packaging of the medical devices. Database records are updated show current physical location and/or ownership based on the read 2D identification codes.

In general, in a sixth aspect, the invention features a computer system. The system has one or more processors, and a machine readable, non-transitory memory having stored therein instructions. The instructions are programmed to cause the processor(s) to store information relating to specific individual medical devices, including physical location and/or ownership. An optical reader reads 2D identification codes on packaging of the medical devices. Database records are updated to show current physical location and/or ownership based on the read 2D identification codes.

Embodiments may include one or more of the following features. A computer may read a machine-readable serial number of a endoscope as the endoscope is put into use. Based on the reading of the serial number, an on-hand inventory level of a class of endoscopes may be reduced and recorded. A reorder level may be computed based on the reduction in on-hand inventory level. A computer may compute when on-hand inventory level warrants reordering to replenish the on-hand inventory. Based on the computed warrant to reorder, a computer may place an order with a supplier of endoscopes of the class to replenish on-hand inventory of endoscopes of the class. The medical device may be an endoscope. The endoscope may have an image sensor. The image sensor may be designed to gather photons to produce video. The image sensor may be of a type that has properties that vary from one sensor to another within manufacturing tolerances. The programs may be programmed to connect a one of the medical devices or endoscopes to a computer processor. The processor may be programmed to obtain properties of the connected medical device from a database. The database may be designed to store information relating to specific individual medical devices, endoscopes, or image sensors. The database may be accessed based on the read serial number. The database may store properties of the endoscope's image sensor. The database may store properties of the image sensor's behavior in a database. The database may store properties specific to specific sensors. The database may provide properties of the one endoscope's image sensor from the database, and to compute normalized video based on the obtained properties and the video from the image sensor. The database may store physical location of individual medical devices, endoscopes, or components. The database may store physical location and/or ownership of individual medical devices, endoscopes, or components. An optical reader may read identification codes on packaging of the medical devices. Database records of physical location and/or ownership may be updated to show current physical location and/or ownership based on the read identification codes. When a medical device or endoscope may be connected to its control computer, the computer may be programmed to obtain properties of the connected device from a database. The identification code may be a machine-readable 2D optical code. The identification code may be stored in machine-readable electronic memory. The database stores information relating to a device model of a class of medical devices manufactured to be interchangeable. The database may be designed to index to the device model information based on the read identification code of an individual medical device. The database may be designed to store data describing a color spectrum capability of the image sensor. The database may be designed to store data describing an image plane resolution of the image sensor. The database may be designed to store calibration properties of the endoscope's image sensor specific to the specific image sensor of the specific endoscope. The image processor may be programmed to compute normalized video based on the obtained calibration properties and the video from the image sensor. The calibration properties may describe a white balance of the image sensor, or a color correction gamma curve of the image sensor, or a distortion correction of the image sensor, or any two or more, or any three or more of these properties. The computer may compute a prediction of delivery date based on the read 2D identification codes. The computer may update the physical location and/or ownership database records to show current physical location based on the read 2D identification codes or read of the machine-readable memory.

The above advantages and features are of representative embodiments only, and are presented only to assist in understanding the invention. It should be understood that they are not to be considered limitations on the invention as defined by the claims. Additional features and advantages of embodiments of the invention will become apparent in the following description, from the drawings, and from the claims.

### DESCRIPTION OF THE DRAWINGS

FIGS. 1A to 1H, 1J, 2A to 2Q, 3A to 3C, 3E to 3G, 4C to 4Q, 5C to 5J, 5L to 5P, 6B, 6D to 6F, 7A, 7C, 7E to 7Z, 8F, 9A to 9D, 9J, 10A, 10D to 10O, 10Q, are perspective or perspective cutaway views of endoscopes and/or endoscope related apparatus.

FIGS. 3D, 4A, 4B, 5A, 5B, 5K, 6A, 6C, 6G to 6J, 7B, 7D, 8A to 8E, 9E to 9I, 9K, 9L, 10B, 10C, 10P, 11E are plan, plan section, or plan partially cut away views of endoscopes and/or endoscope related apparatus.

FIGS. 11, 11A to 11D, 11G, 12E, and 12H are block diagrams of computers or processors.

FIG. 11F is a time sequence of video frames.

FIGS. 12A-12D, 12F, and 12G are screen shots.

### DESCRIPTION

The Description is organized as follows.
I. Overview
   I.A. Endoscopic surgery
   I.B. Overall architecture
   I.C. Integrated sterile packaging
   I.D. Single-use handpiece
II. An endoscope that is partially-reusable, partially disposable/replaceable, and a coupling joint between
III. Extendable, bendable, or articulated camera tip
IV. Additional features of an endoscope
V. Endoscope tip
   V.A. Molding and assembly of components of the endoscope tip
   V.B. Fiber optic illumination for a single-use use scope tip
   V.C. A tip design with light guides
   V.D. Diffusion terminal surface
VI. Antifouling and antifogging
   VI.A. Heating
   VI.B. For endoscope's delivery packaging, vial of fluid to protective a coating on the endoscope's lens/window
VII. Lens cap with optical correction for use during insertion of offset-view endoscope
VIII. Avoiding fasteners, springs, and other small components
   VIII.A. A button with embedded springs
   VIII.B. Overmolding of case over circuit board
   VIII.C. Avoiding internal fasteners
   VIII.D. Rotational resistance via O-rings
   VIII.E. Ultrasonic welding of the two halves of the outer handle shell
   VIII.F. Thermoplastic elastomer coating handle
IX. Liquid flow
   IX.A. Liquid-tight seal
   IX.B. Inducing spiral flow
X. Molding and Joining
   X.A. Diagonal slots to connect dissimilar materials
   X.B. Joining component parts of obturator
   X.C. Twist-locking the parts together
XI. Endoscope tip
   XI.A. Molding and assembly of components of the endoscope tip
XII. Image processing unit
   XII.A. Image processing
   XII.B. HDR exposure fusion to preserve frame rate
   XII.C. Auto Exposure
   XII.D. Video processing for superresolution
   XII.E. Diagnosis and lesion detection
   XII.F. Scope control
   XII.G. Flexboard and electronics in the endoscope handle
   XII.H. Cable
   XII.I. Wireless communication in place of cable
   XII.J. Isolation
   XII.K. Other peripherals
      XII.K.1. Monitor
      XII.K.2. USB port
      XII.K.3. Connections to cloud storage
      XII.K.4. USB connection for keyboard and mouse
      XII.K.5. Microphones
      XII.K.6. Insufflation tubing
XIII. Machine-readable serial number
   XIII.A. Machine-readable serial number
   XIII.B. Use of machine-readable serial number to reduce errors, ensure non-expiration and ensure sterile single-use
   XIII.C. Use of machine-readable serial number to communicate patient data into electronic medical record
   XIII.D. Use of machine-readable serial number for inventory control, location tracking, reordering, and stock management
   XIII.E. Configuration/capability information
   XIII.F. Calibration information
   XIII.G. Implementation
XIV. Embodiments

### I. Overview

### I.A. Endoscopic Surgery

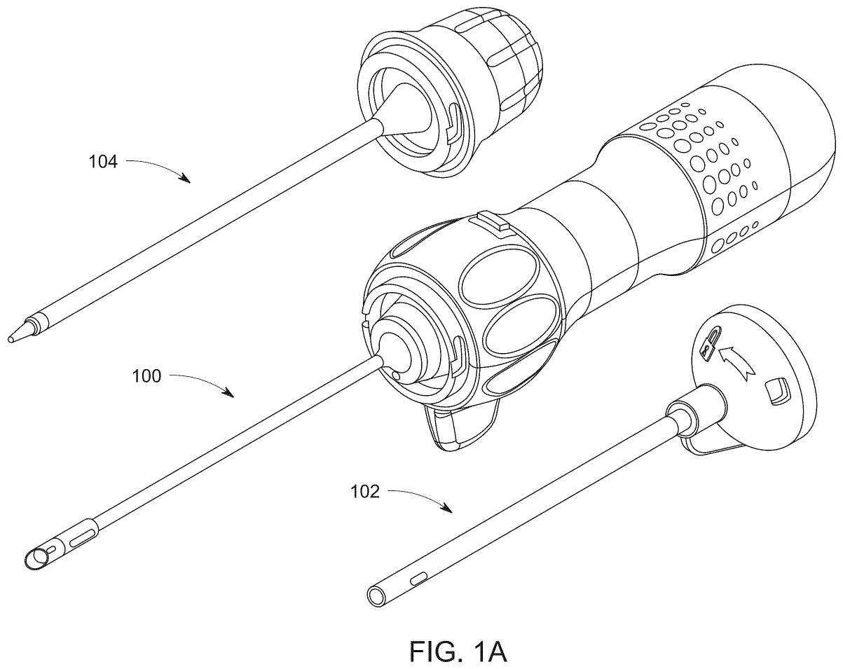

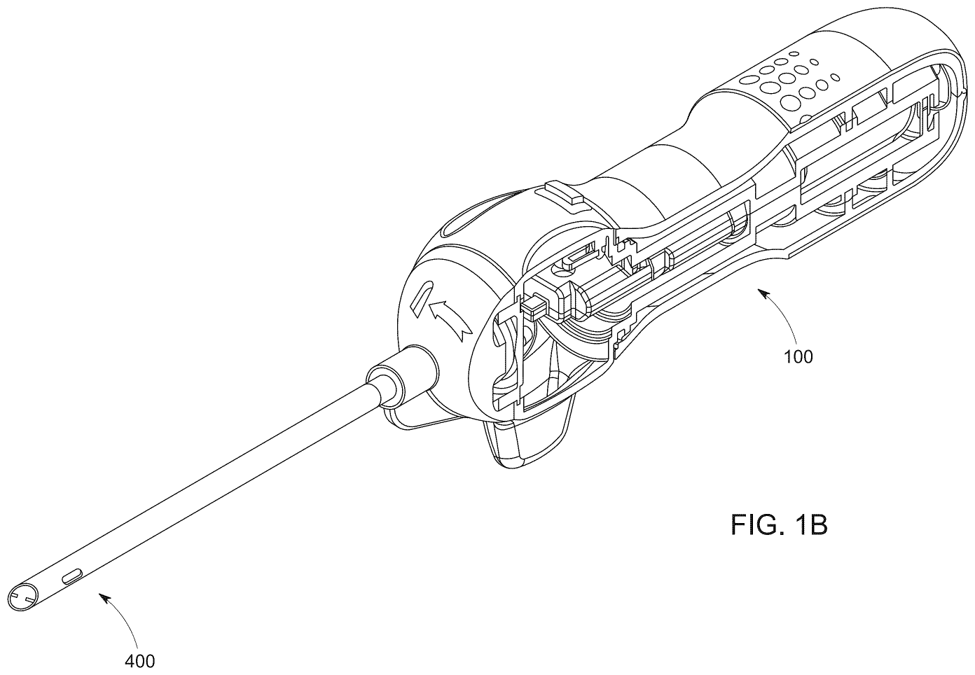

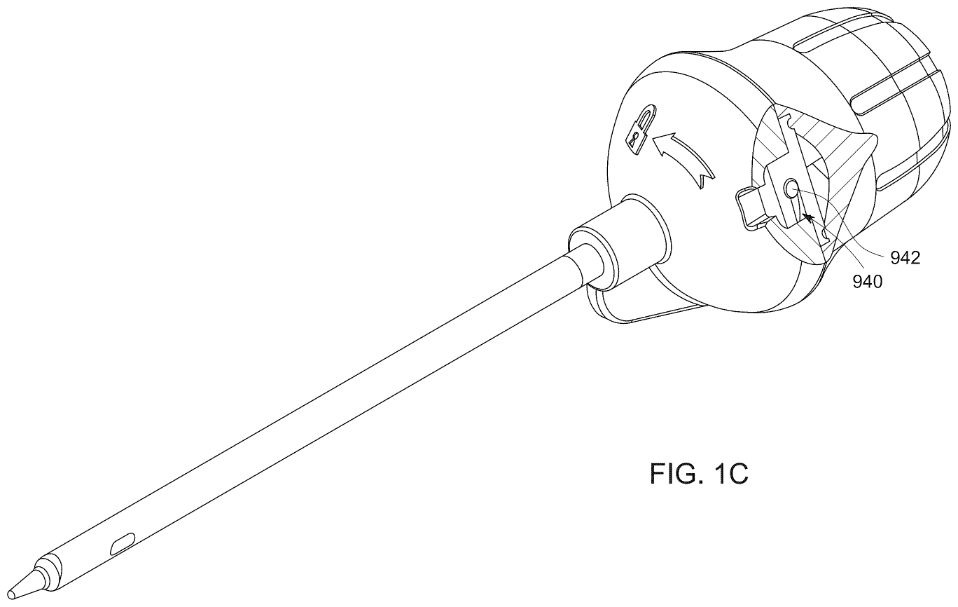

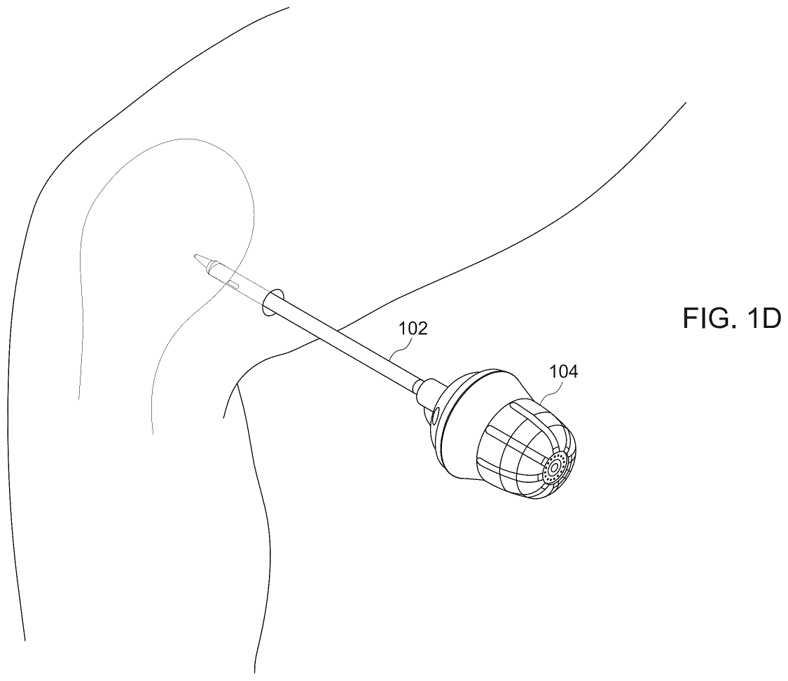

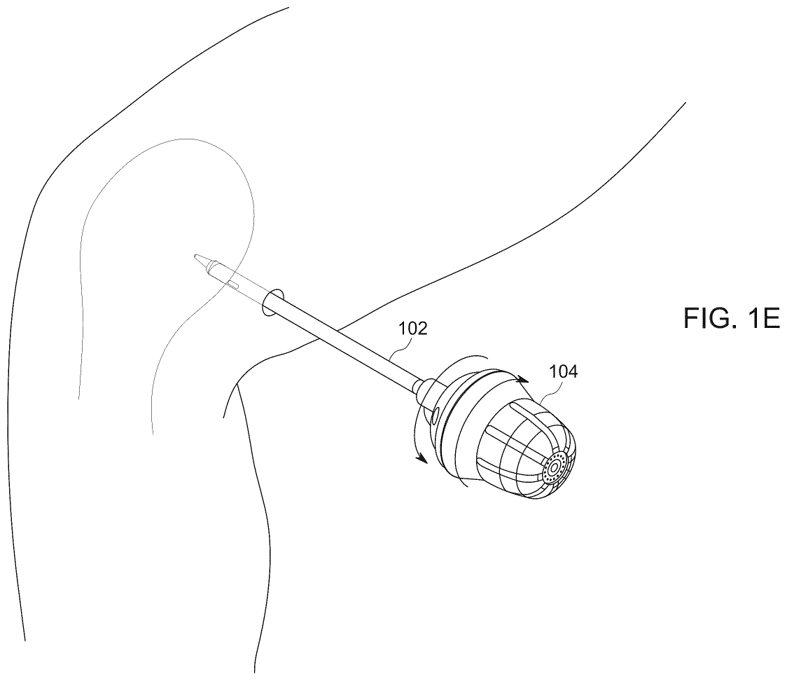

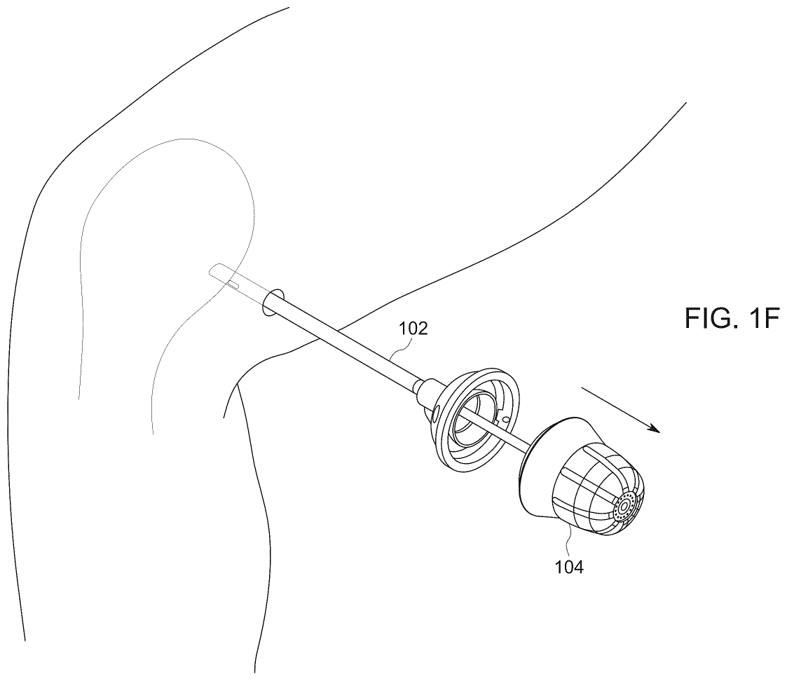

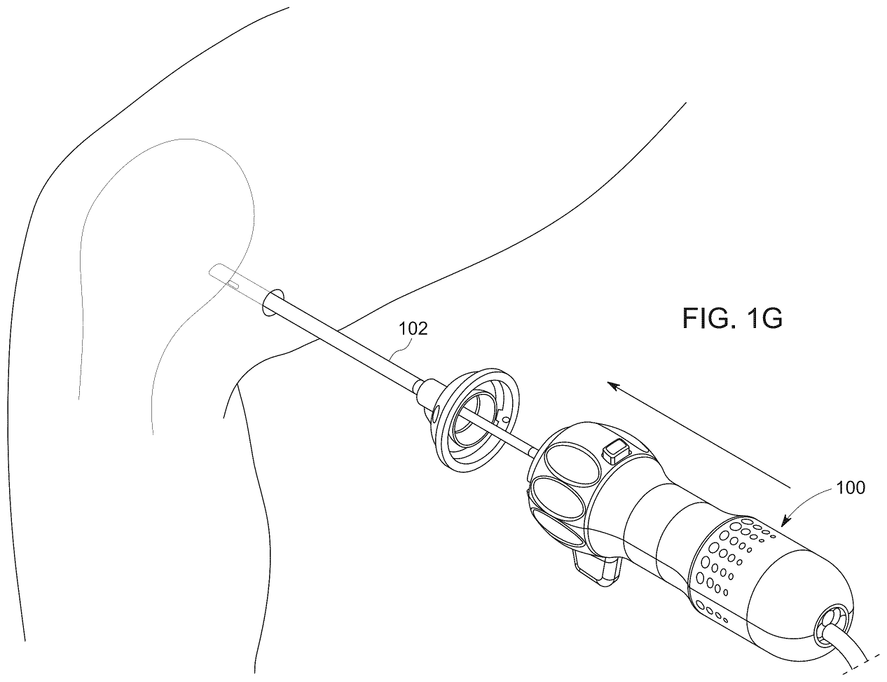

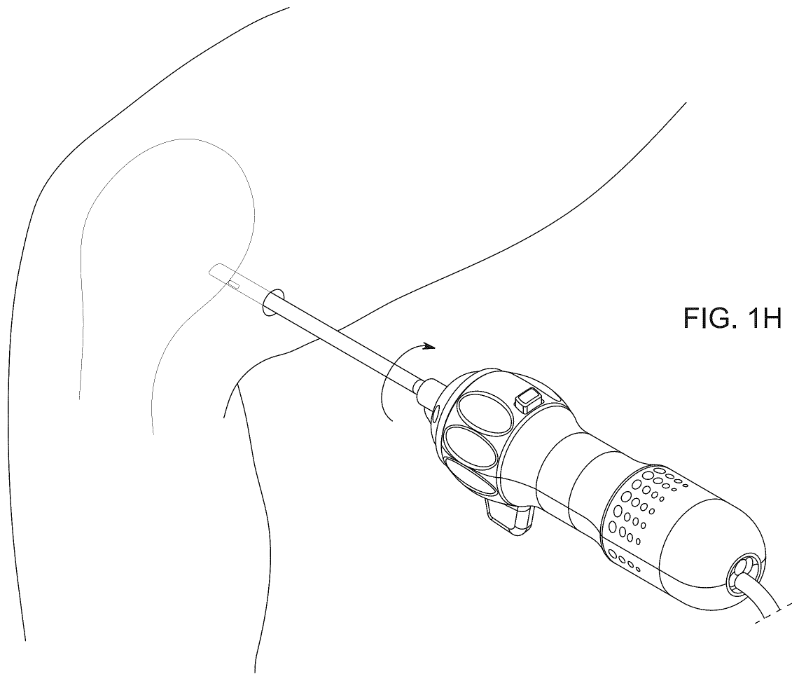

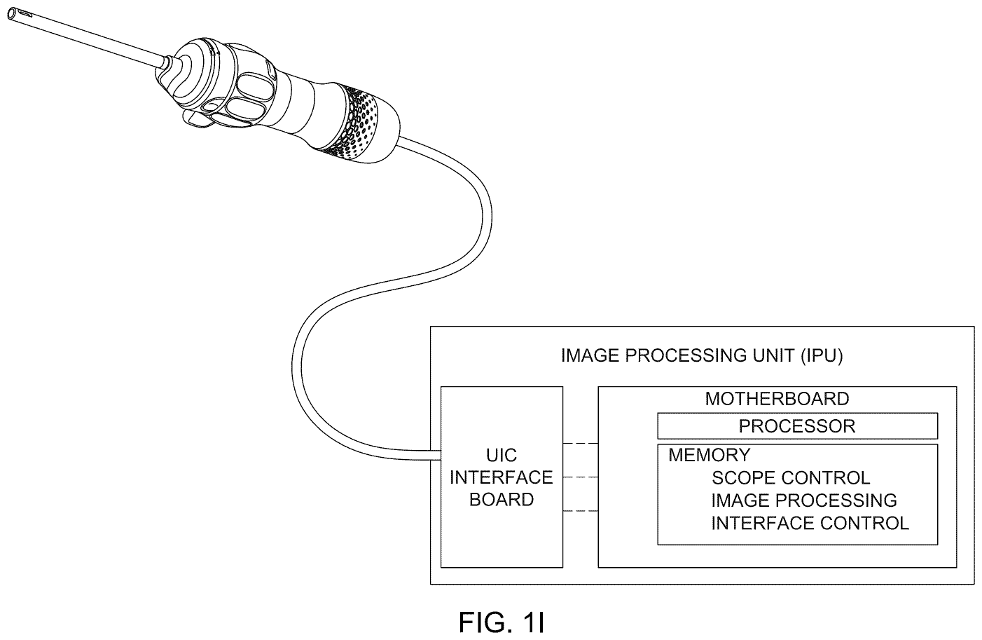

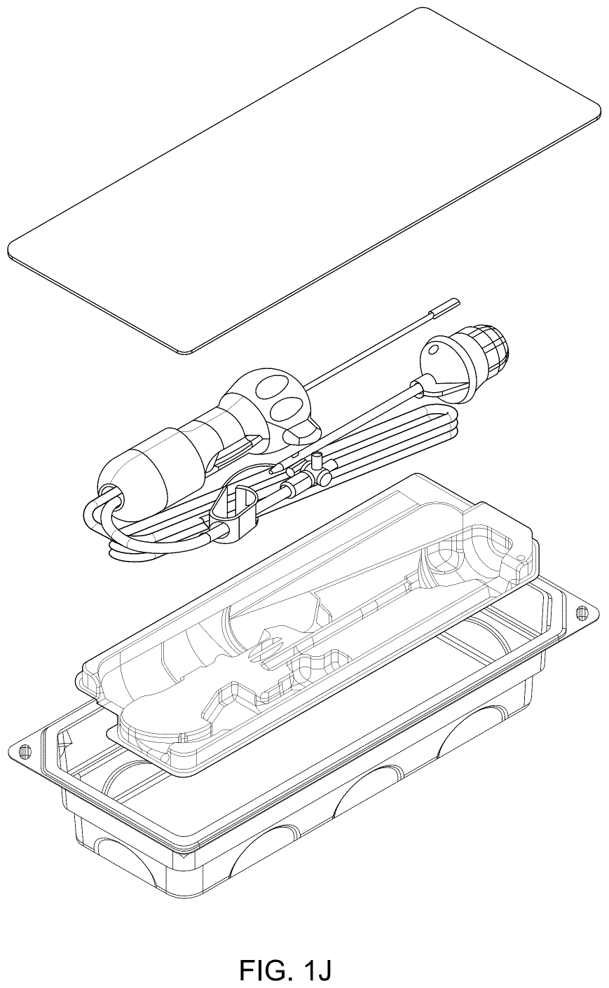

Referring to FIG. 1A, endoscope 100 (such as an arthroscope, laparoscope, or other), trocar 102, and obturator 104 may be used for joint surgery, joint access, or other minimally-invasive surgery. A minimally-invasive surgery generally begins with penetration to the surgical site using a pointed instrument such as trocar 102 or obturator 104. Trocar 102 is a hollow tube used to pierce from the skin to the surgical site; trocar 102 will remain in place during the surgery to maintain an access port. During piercing, trocar 102 may have obturator 104 inserted through the lumen. Obturator 104 and/or trocar 102 may have sharp points for piercing into the tissue of a joint, abdominal cavity, or other surgical site to create an access portal. Obturator 104 may be inserted into trocar 102, used as a unit to pierce skin and other tissues, and once the space of interest is accessed, obturator 104 may be pulled out, leaving the “trocar cannula.” The scope may be placed through the cannula for visualization of the surgical site. The endoscope may be a “chip on a tip” scope, having an illumination source and camera 410. After an access portal is cut via obturator 104 and trocar 102, the endoscope may be inserted through trocar 102 to the surgical site, to provide the visual access to a surgeon who performs the surgery with other instruments. Other trocars may placed for access by other instruments, such as laparoscopic scalpels, shavers, staplers, suture machines, and the like.

In some cases, endoscope 100 may be inserted down the inner lumen of obturator 104, and the point of obturator 104 may be transparent. The endoscope-within-obturator configuration may provide visual guidance to guide obturator 104 or trocar 102 to the correct surgical site. In other cases, trocar 102 may have a sharp point, and the endoscope may be inserted down the inner lumen of trocar 102, to guide trocar 102.

Referring to FIG. 1B, endoscope 100 may have a number of features that ensure reliability, reduce manufacturing costs to the point that the entire endoscope, or portions thereof, may be disposable. Resterilization of endoscopes is costly, and always imperfect, which increases risk of cross-infection. Also, as endoscope 100 is reused, the optics become scratched and clouded, reducing the precision of the view available to the surgeon. Sections II to X will discuss multiple features of the endoscope that provide such cost reduction and disposability.

Referring to FIG. 1C, obturator 104 may be locked into the outer sheath or trocar 102 via a twist lock 940, 942.

Referring to FIG. 1D, obturator 104 and trocar 102 may be used to pierce into the surgical site, for example, a region of a knee behind the patella.

Referring to FIGS. 1E and 1F, obturator 104 may be unlocked from outer sheath/trocar 102. Obturator 104 and endoscope may be withdrawn, leaving hollow trocar 102 as a port to the site.

Referring to FIG. 1G, instruments, such as endoscope 100, etc. may be inserted through the outer sheath/trocar 102 to perform the surgery.

Referring to FIG. 1H, trocar/sheath 102 may be locked to the endoscope via the same twist lock motion as was used to hold the outer sheath/trocar 102 to obturator 104.

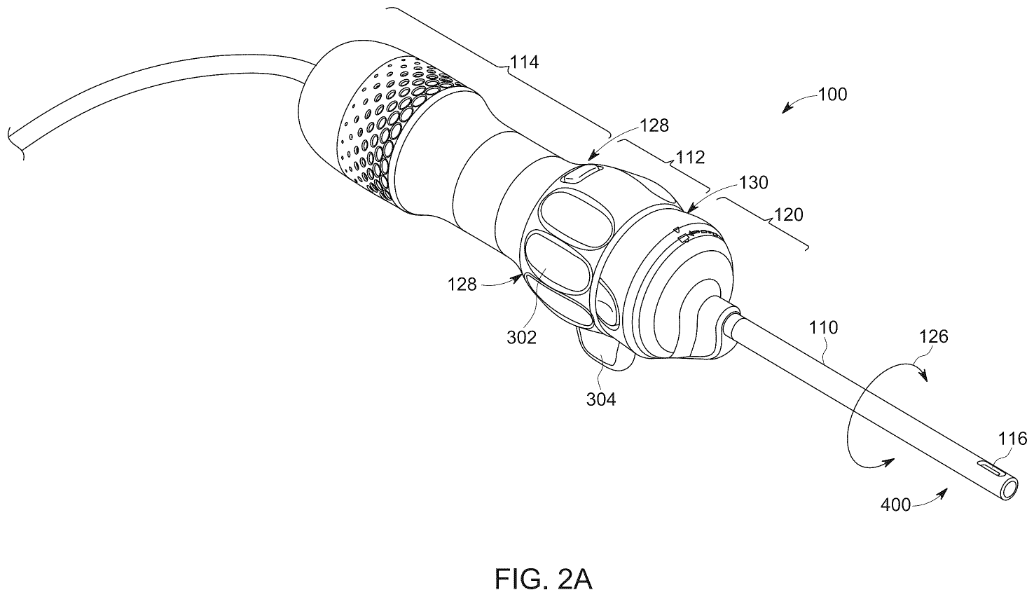

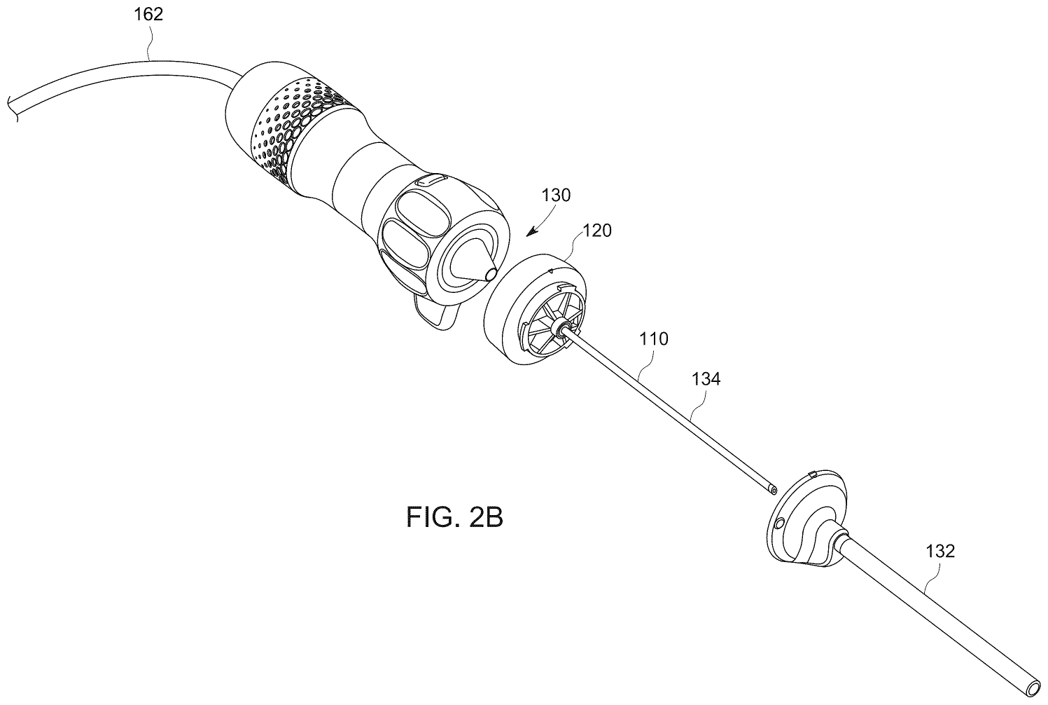

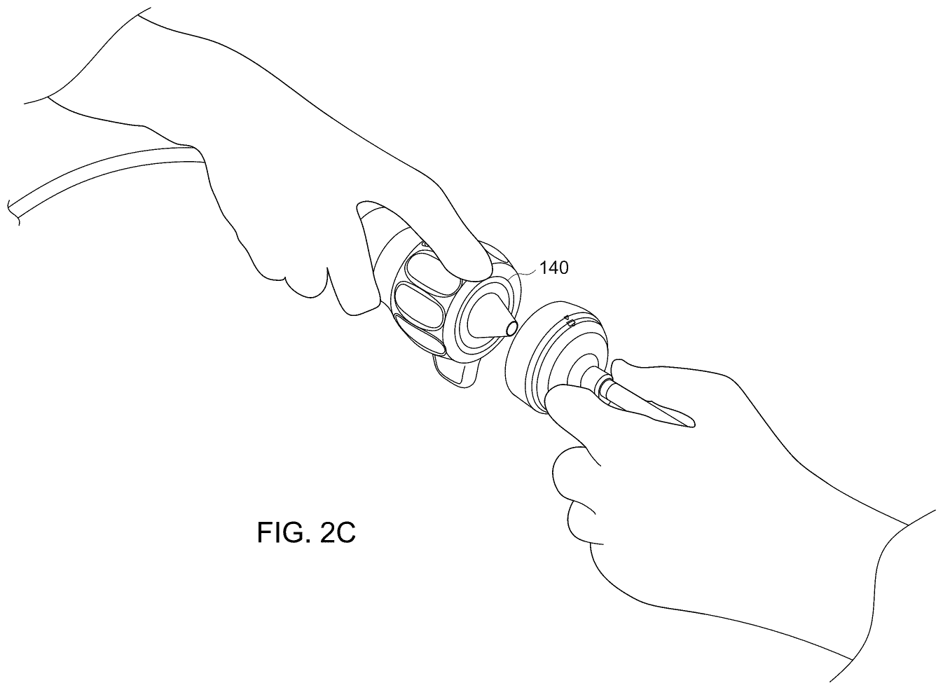

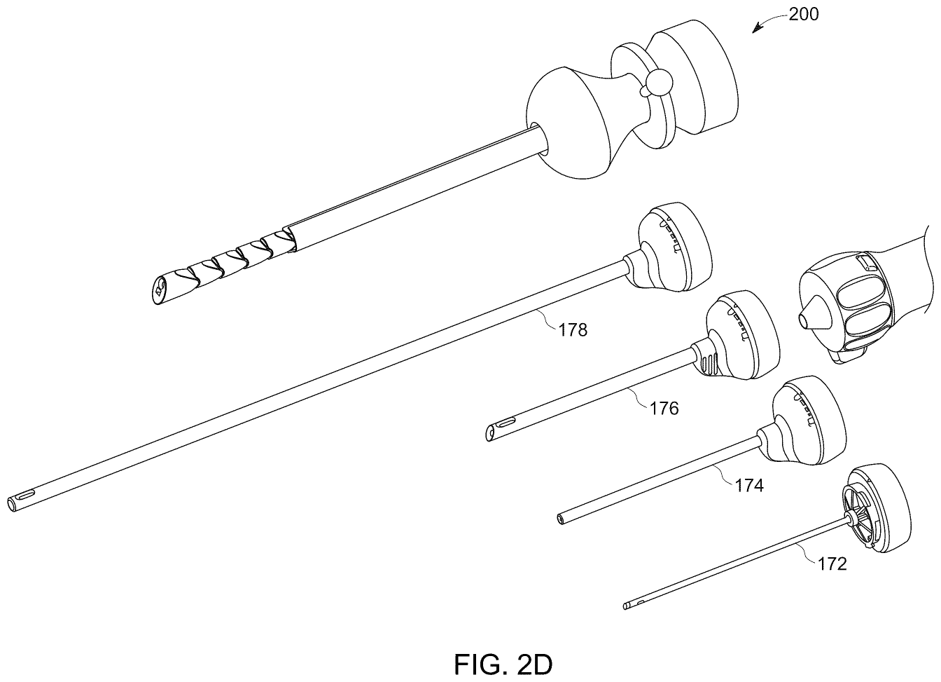

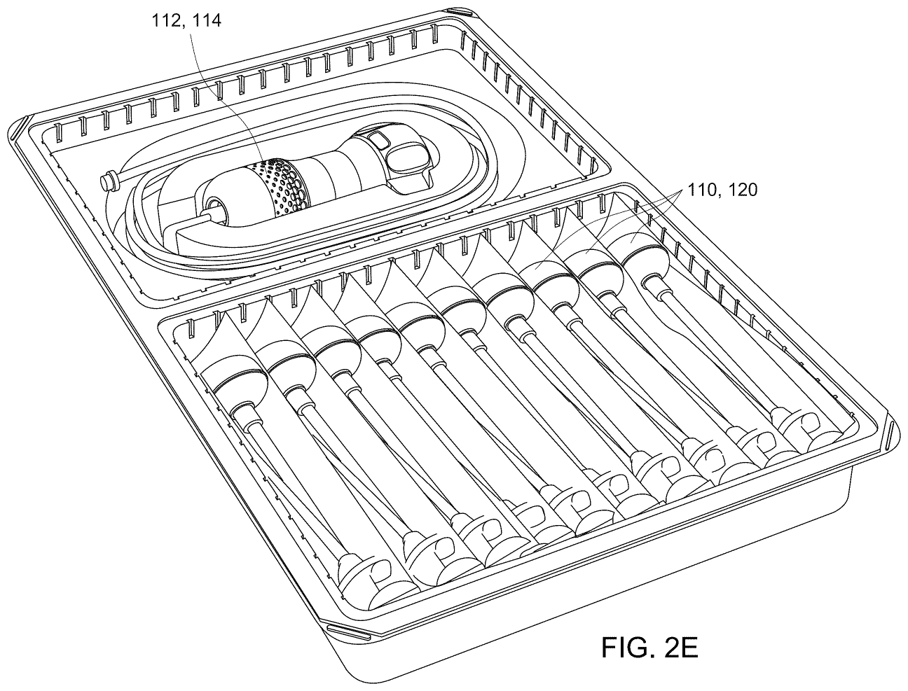

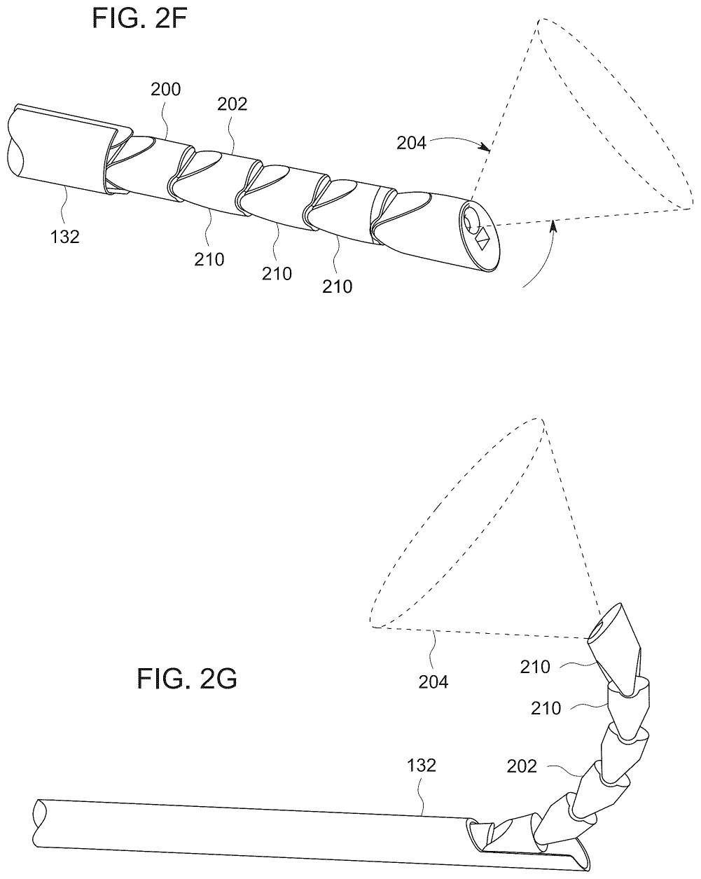

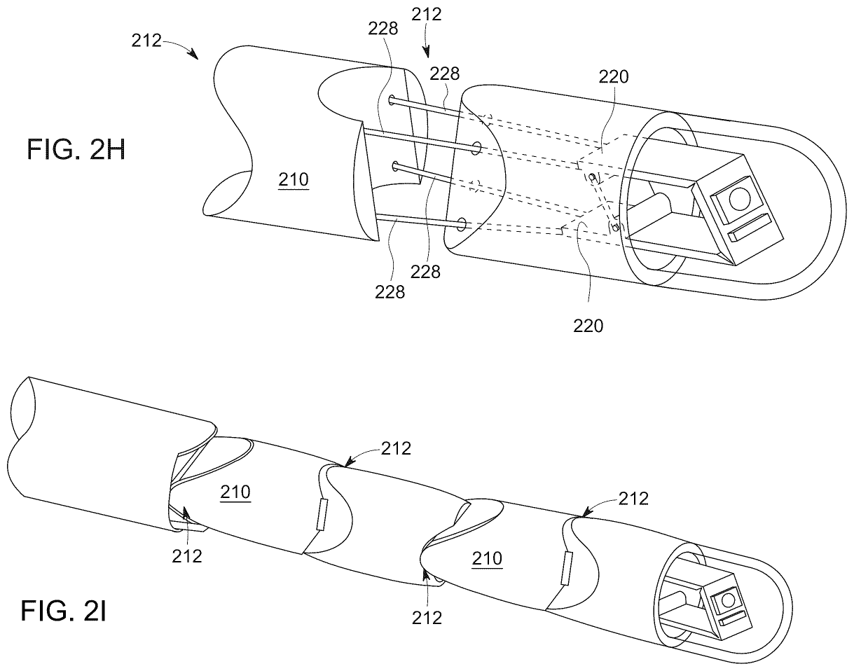

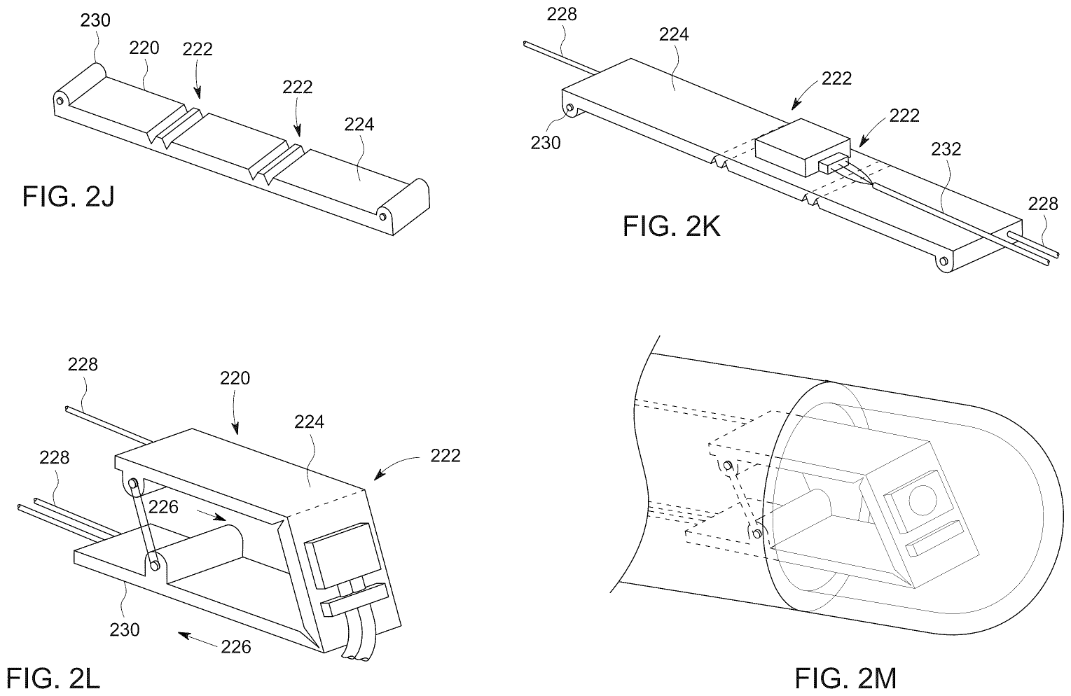

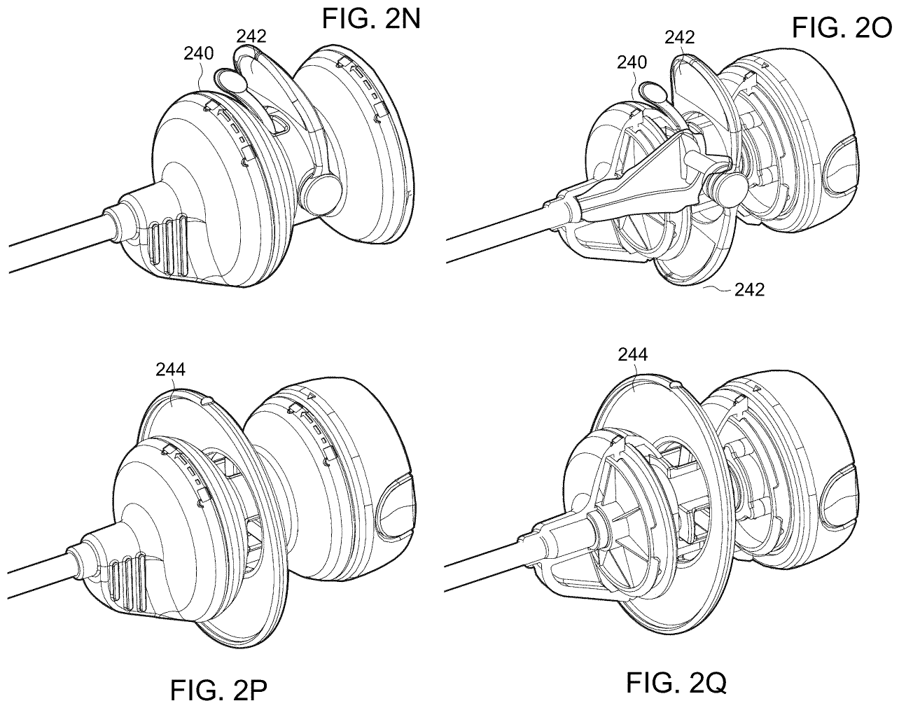

The various endoscope tip designs (FIGS. 2F-2M, 4A-4K, 4M-4A, and FIGS. 10A to 10Q), may have the following properties. The overall tip may be small enough to meet the dimensions of the endoscope, typically the dimensions in the table of paragraph [0040] below. In some cases, the tip may be slightly larger or smaller in diameter than shaft The tip may hold camera 410, illumination, fluid injection or evacuation ports, procedural tools, etc. mechanically stable, within that diameter. The tip may seal against elevated pressures that are typically used to distract tissues out of the view of the scope, to prevent intrusion of bodily tissues and fluids and insufflation fluid. The tip may deliver or allow delivery of illumination light, either via an LED 418 mounted in the tip, or using fiber optics 430 to convey light from the handle or a controller. Opaque parts of the tip assembly may exclude stray light, from non-desirable lights paths within the tip from the illumination fibers/LEDs/light guides, and reflected light from the surgical cavity. The tip may be manufacturable at desired quantities and cost. The tip may have a configuration that is atraumatic to surrounding tissue, for instance, without sharp points or edges. The scope may be formed of biocompatible materials, such as stainless steel and/or certain plastics. In some cases, the tip may have a piercing point. The tip may be designed to resist fogging or fouling. The tip may permit cleaning, preferably while in situ the surgical site.

### I.B. Overall Architecture

Referring to FIGS. 11, 1J, and 10A, endoscope 100 may be part of an overall system designed to deliver high-definition video for use in endoscopic surgeries. The system may provide live high-definition video to be displayed on a video monitor, and to be captured as stored video and still images; illumination of the surgical cavity, irrigation and/or inflation (insufflation) of the surgical site, and image refinement such as zoom, rotation, removal or reduction of hotspots and other artifacts, etc.

The system may include an endoscope, including insufflation tubing, a communications/control/power/illumination cable, a cannula, and an obturator. An image processing unit (IPU) or master controller may be reusable over multiple procedures. If illumination is provided via fiber optics, there may in addition be a light box, typically near the IPU so that the fiber optics fibers are aligned with the other necessary cords and hoses. One or more of the endoscope, tubing, cable, cannula, and obturator may be designed for disposable single use, and sold together as an integrated kit.

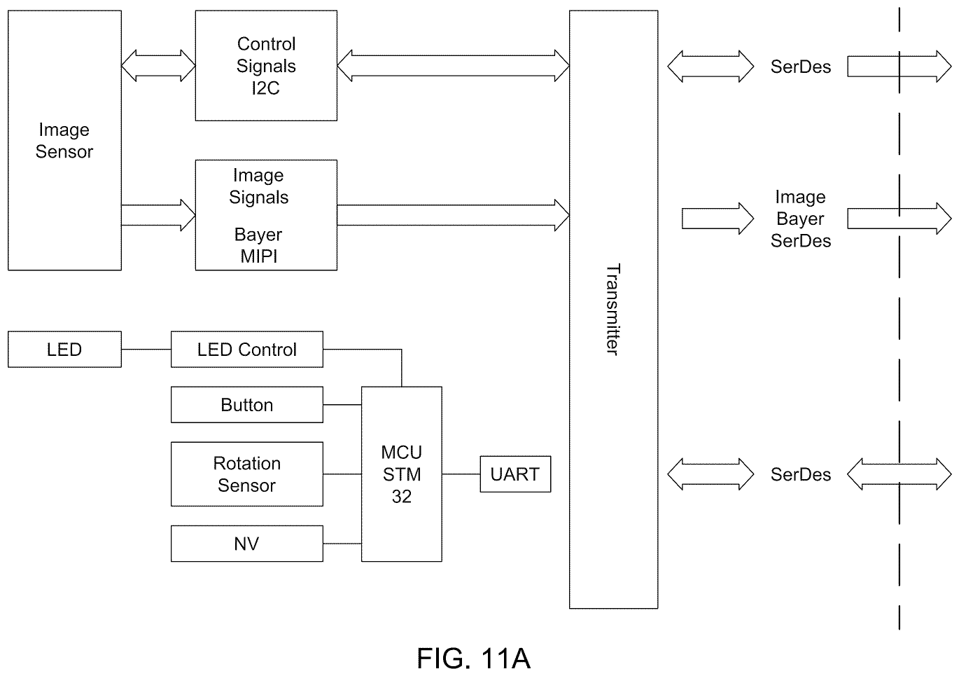

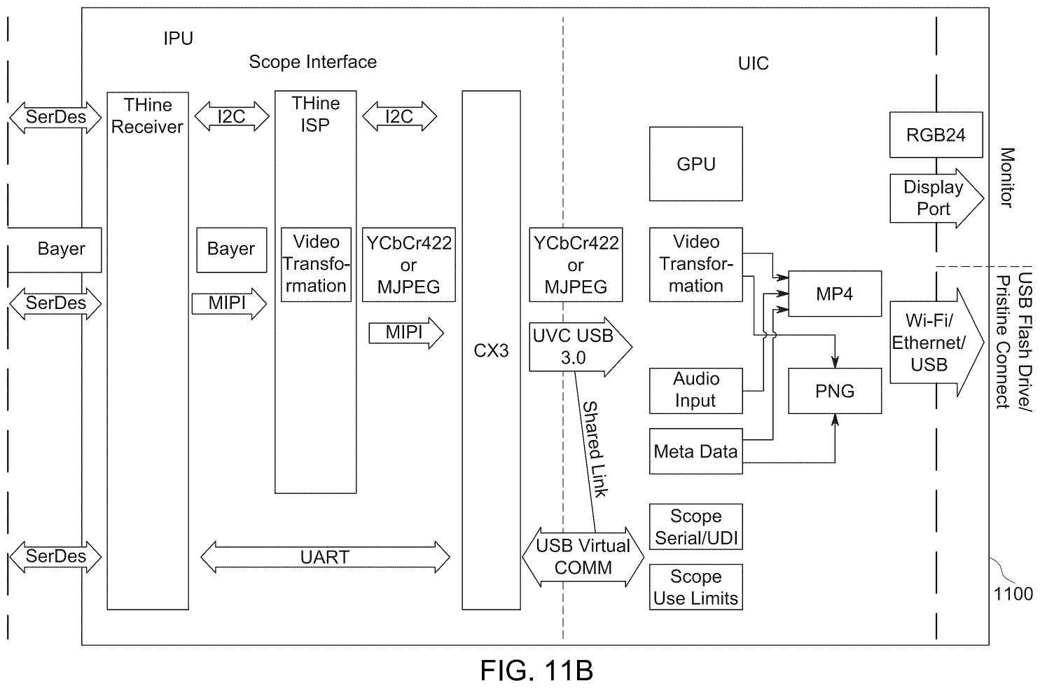

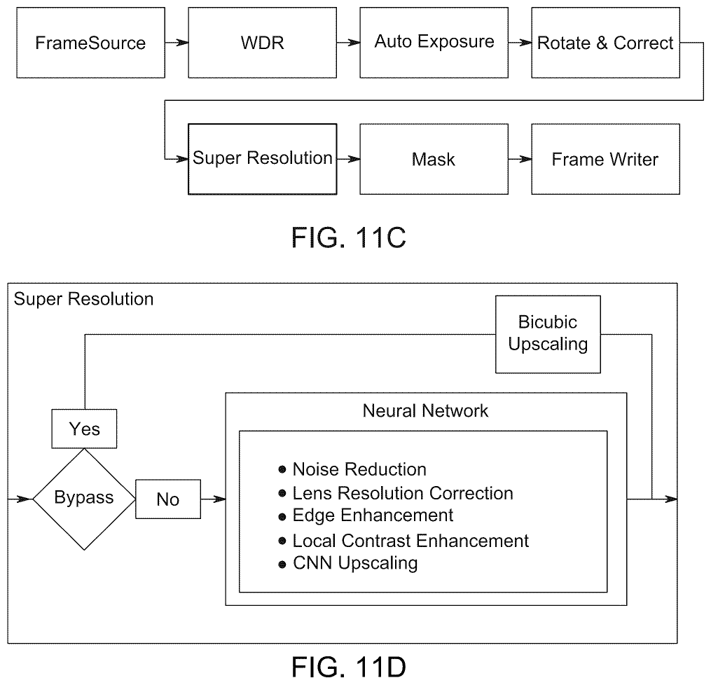

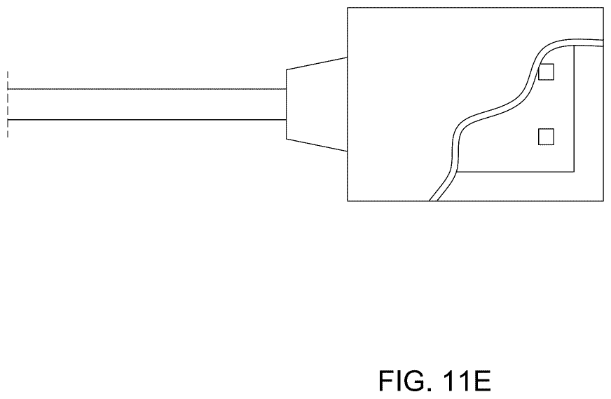

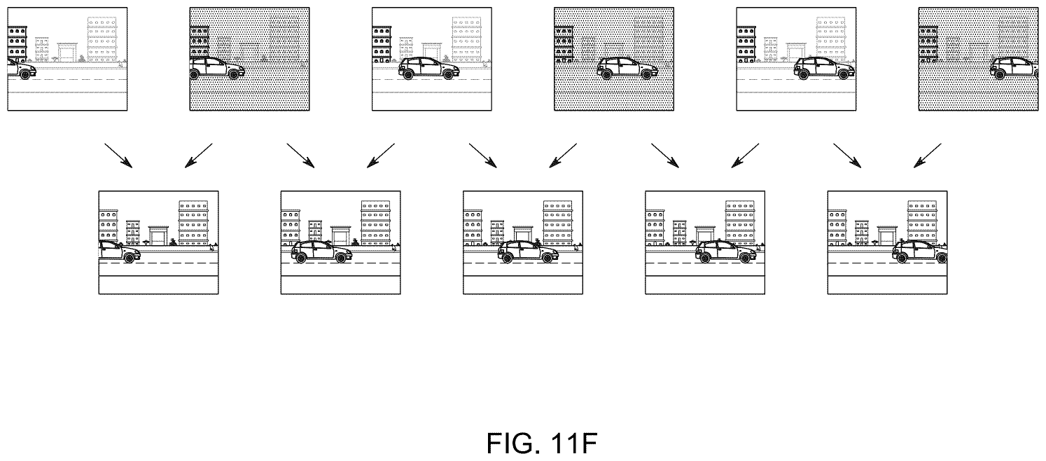

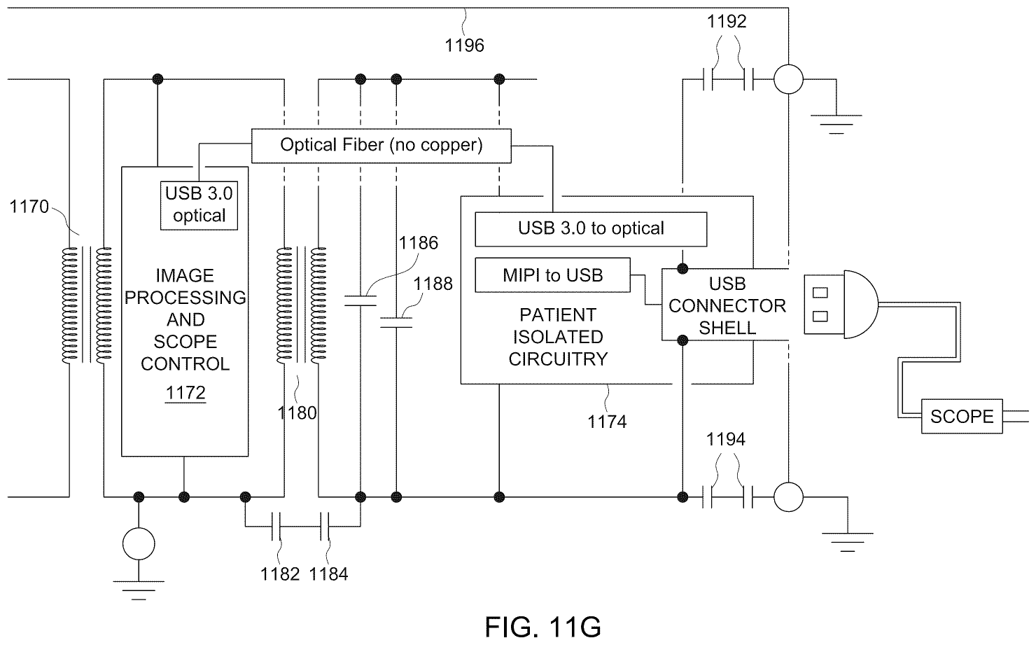

Referring to FIGS. 11A and 11B, the endoscope may have electronics in the handle that controls the camera and illumination (LED or fiber optics). The IPU may have a computer processor for various image processing functions, and controllers for the electromechanical devices in the endoscope, Wi-Fi or similar radio communication, USB and cloud storage, and the like. Because the scope is single-use, sterility is easily provided. The connecting cable may be single-use as well, so that it can be delivered in the sterile packaging. The IPU is higher cost, and cannot easily be sterilized, so it is outside the sterile field.

Referring to FIG. 11G, various isolation couplers may provide electrical isolation between the wall-voltage components for the IPU and the patient.

### I.C. Integrated Sterile Packaging

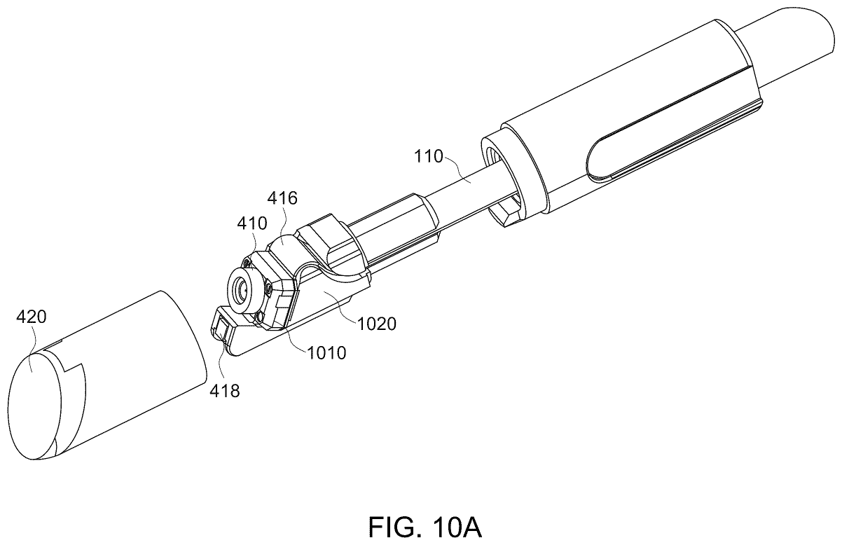

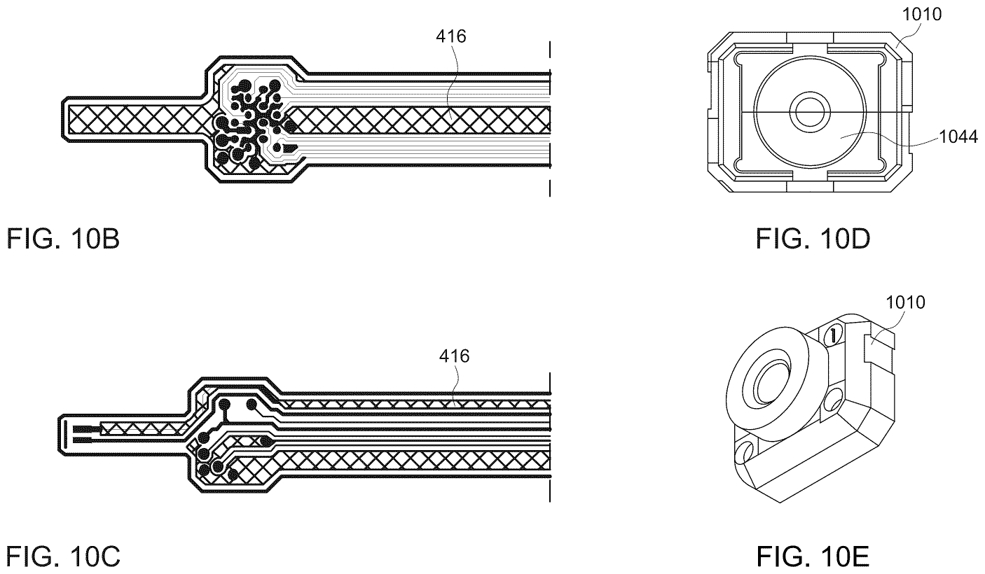

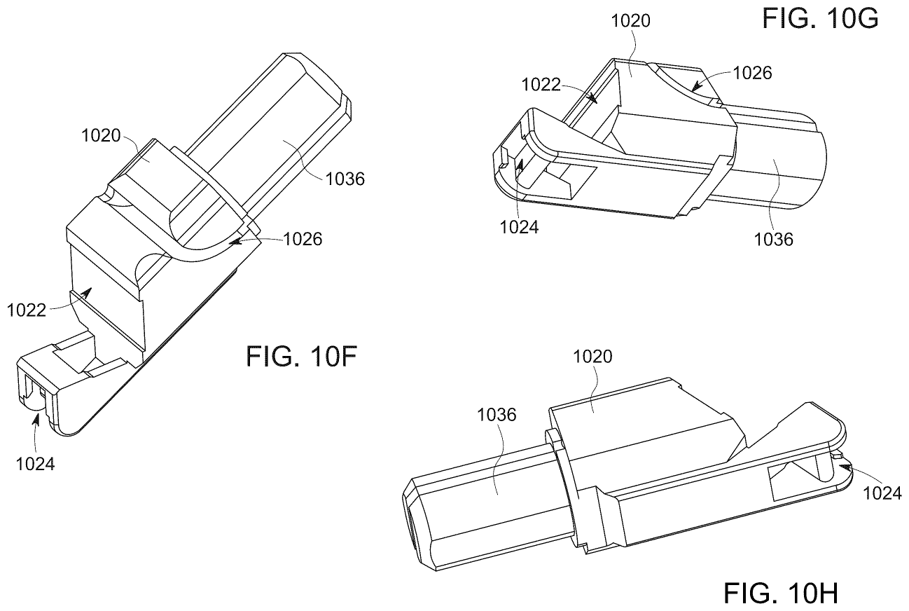

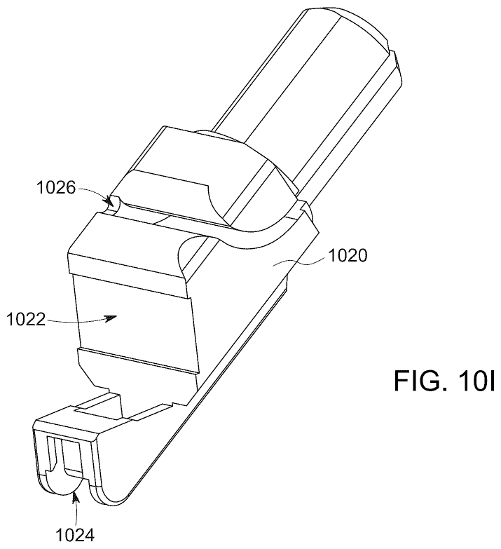

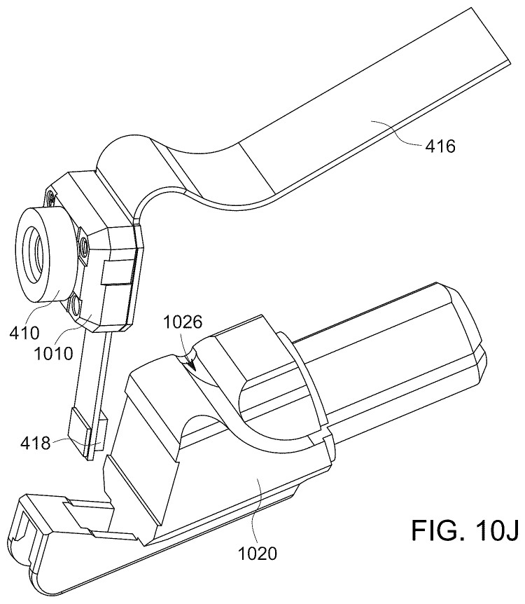

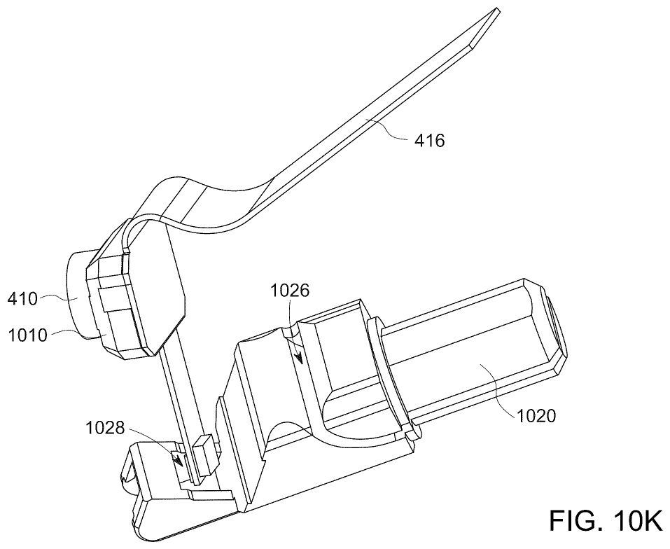

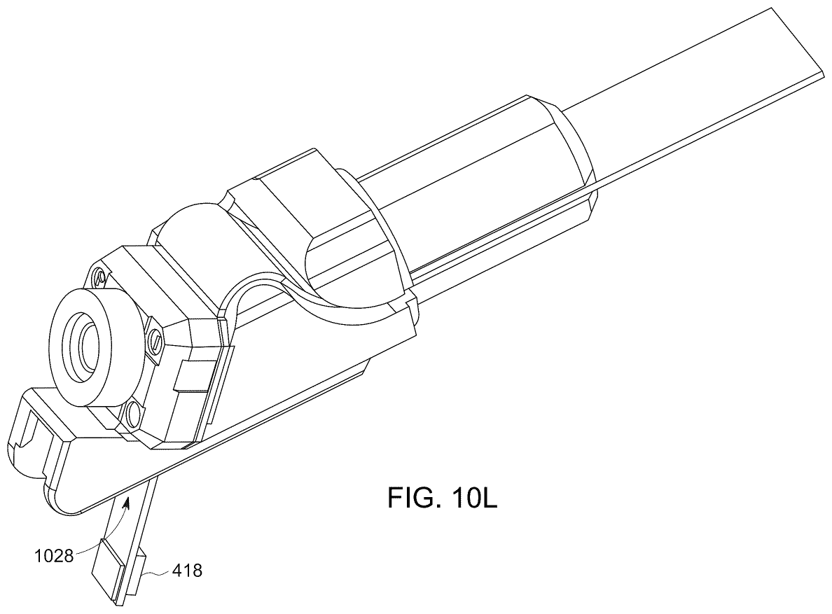

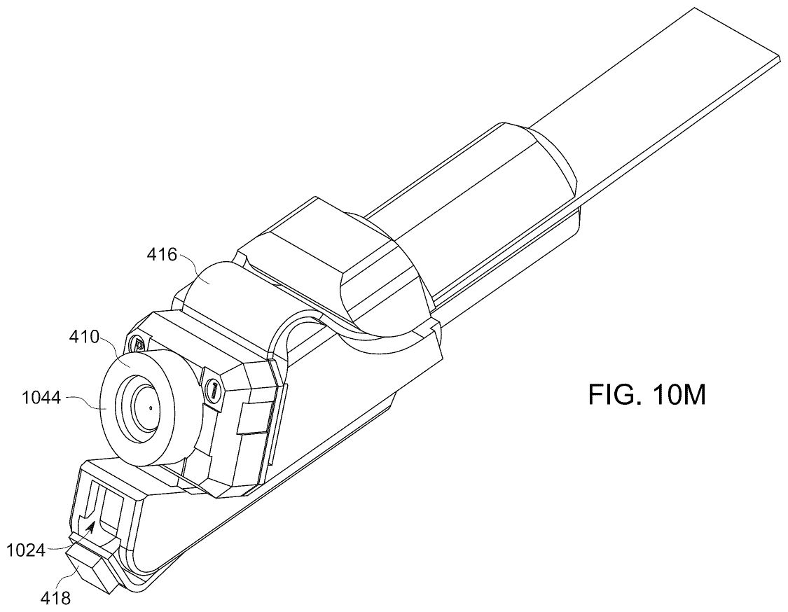

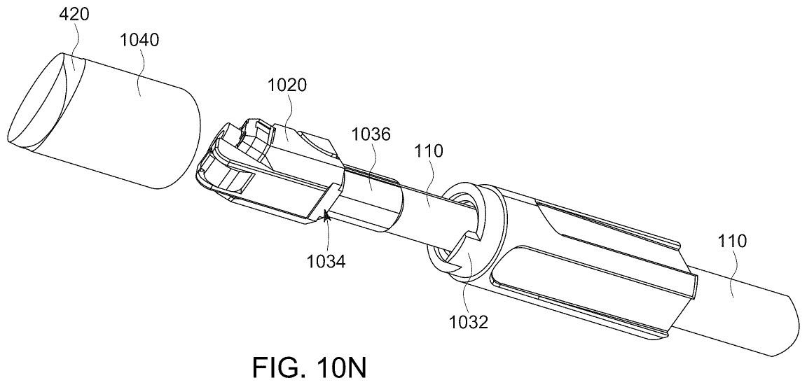

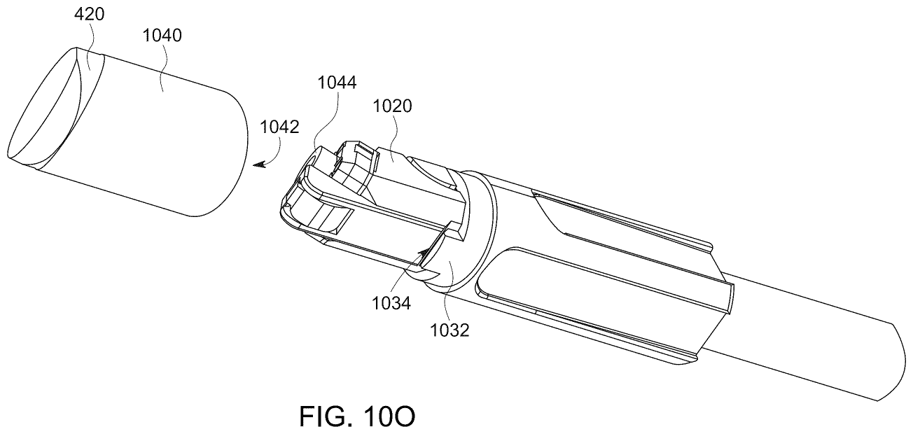

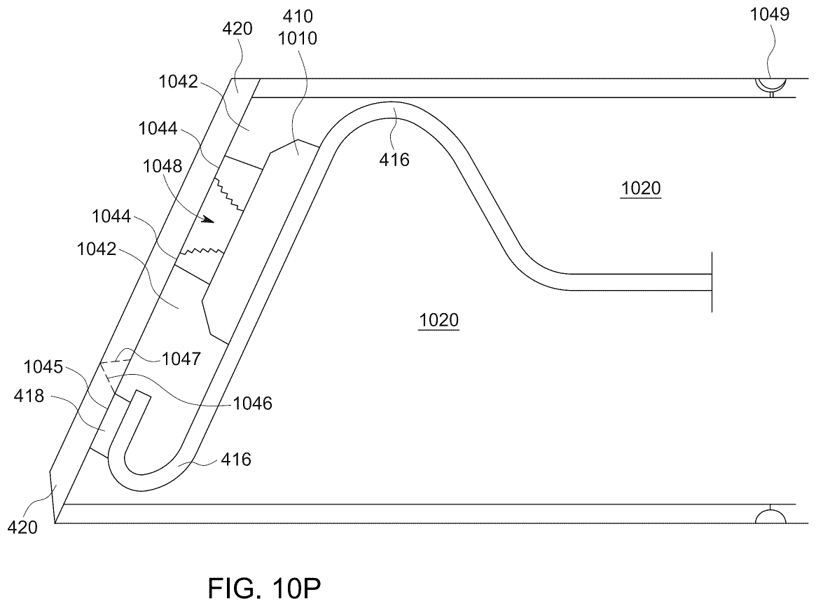

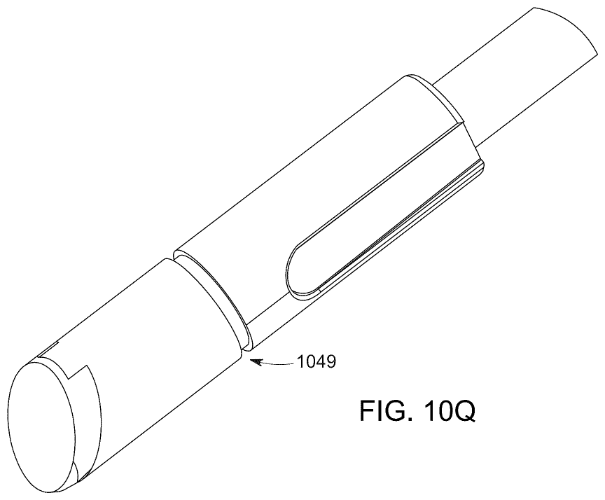

Referring again to FIGS. 1J and 10A, the endoscope, tubing, and cable may be designed for disposable single use, and packaged and sold together as an integrated kit. Additionally, one or more of the obturator and cannula may be packaged and sold together with the kit. The kit may be sold in sterile packaging. The packaging may be designed to be opened at the surgery, within the sterile field surrounding the patient. The cover on the packaging may be made of Tyvek® or some similar film that is transparent to ethylene oxide or a similar sterilant, so that the packaging and components may be sterilized together at the time of manufacture. The film covering stays in place until shortly before the surgery. This eliminates the need to disinfect or sterilize the scope immediately before surgery. The tray holding the components may be transparent, so that the contents of the tray are visible before the Tyvek cover is opened.

Because the components are sold together, they can be calibrated to each other. Various properties of the illumination, image sensor, lens, filter, and the like can be calibrated to each other as a set at the manufacturing plant. White balance may be one of the parameters calibrated at the factory-because the components are single use and sold as an integrated package, they can be inter-calibrated at the factory, and that co-calibration follows them for the life of the product. In contrast, for conventional endoscopes, the light source and the endoscope are independent and the color temperature or balance of the illumination source varies from light source to light source, and the color sensitivity of the pixels of the image sensor vary scope-to-scope, so white balance must be performed by the user as part of the prep for each procedure. In a configuration where the scope is sold as a disposable single-use configuration, with an electronic serial number that ties back to calibration factors measured at the factory (see § XIII.A and ¶¶ [0211] to [0218], below), the scope may be calibrated by imaging a white surface, which provides a test surface with equal parts red, green, and blue pigment, with illumination that results in mid-level, non-saturated pixel values from the image sensor and an matrix of correction coefficients may be computed adjust color balance of the pixels of the image sensor's signal.

### I.D. Single-Use Handpiece

The endoscope itself may be designed for disposable single use. The image sensor, a lens, a filter, and cover window, and illumination emitter (either an LED 418 or the distal end of fiber optic lighting fibers or wave guides) may be located at the distal end of an insertion shaft. The sensor, lens, filter, cover window, and illumination emitter may be designed to interoperate with each other to allow insertion in a small diameter insertion shaft. Single use ensures sterility, even of components with complex geometric forms and materials that cannot be autoclaved (like the electronics of endoscopes). The endoscope may have electronic tracking to ensure single use (see § XIII.B and ¶¶ [0219] to [0227], below).

### II. An Endoscope that is Partially-Reusable, Partially Disposable/Replaceable, and a Coupling Joint Between

Referring to FIGS. 2A, 2B, 2C, 2D, and 2E, a surgical endoscope 100 may be structured to permit detachment of a shaft 110 portion from the endoscope's handle 112, 114. Camera or image sensor 410 at tip 116 of the shaft, any panning mechanism, illumination, power and signal connectors, and fluid flow channels may be in the disposable shaft 110. Handle 112, 114 may be designed to be reusable (which implies that handle 112, 114 may be sterilizeable, for example in an autoclave or other sterilization device, or protectable by a disposable sterility sleeve). Joint 130 between the detachable shaft and the reusable parts of handle 112, 114 may be generally distal in the handle (but not necessarily at the distal-most end). The replaceable shaft portion 110 may be disposable, along with a disposable portion 120 of the handle that is disposable with shaft 110.

Referring to FIGS. 2A and 2B, the handle of the endoscope 100 may include three principal components:
The disposable cap 120. This distal-most portion of the handle may serve as a mounting base for shaft 110, and may disconnect from the remainder 112, 114 of the handle. This disposable cap portion 120 (along with shaft 110 and componentry inside) may be disposable.
Rotation collar 112 may have surface features 302, 304 to allow a surgeon to rotate the rotation collar 120 about the central axis of the handle, that is, about the roll axis 126 of shaft 110. During surgery, insertion shaft 110, disposable cap 120 and rotation collar 112 may be locked to rotate with each other, so that rotating the rotation collar effects rotation 126 of the disposable cap 120 and shaft 110.
Proximal stationary handle 114 has a shell surrounding componentry within the handle. The outer diameter and outer surface of handle 114 may be designed to provide an easy and low-slip grip for a surgeon's hand. Joint 128 between the proximal handle and rotation collar may allow these two components to rotate relative to each other. In some cases, a circuit board and similar componentry inside proximal handle 114 may rotate with disposable cap 120 and rotation collar 112, inside proximal handle 114.

Disposable cap 120 and rotation collar 112 may be separable from each other at joint 130, so that disposable cap 120 and shaft 110 may be disposable, while handle 114 and rotation collar 112 (and componentry inside them) are reusable.

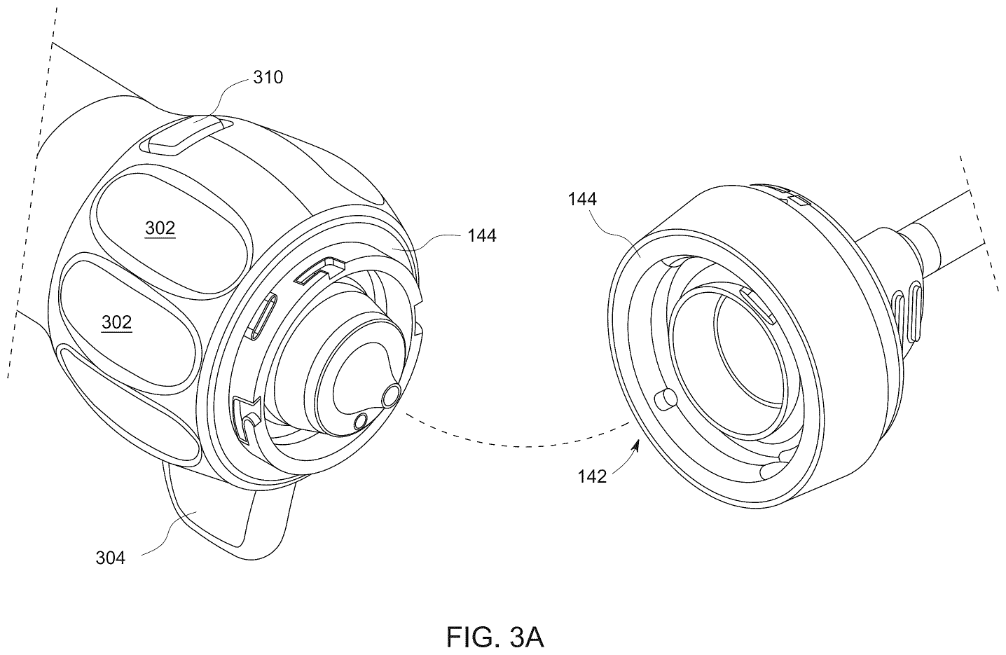

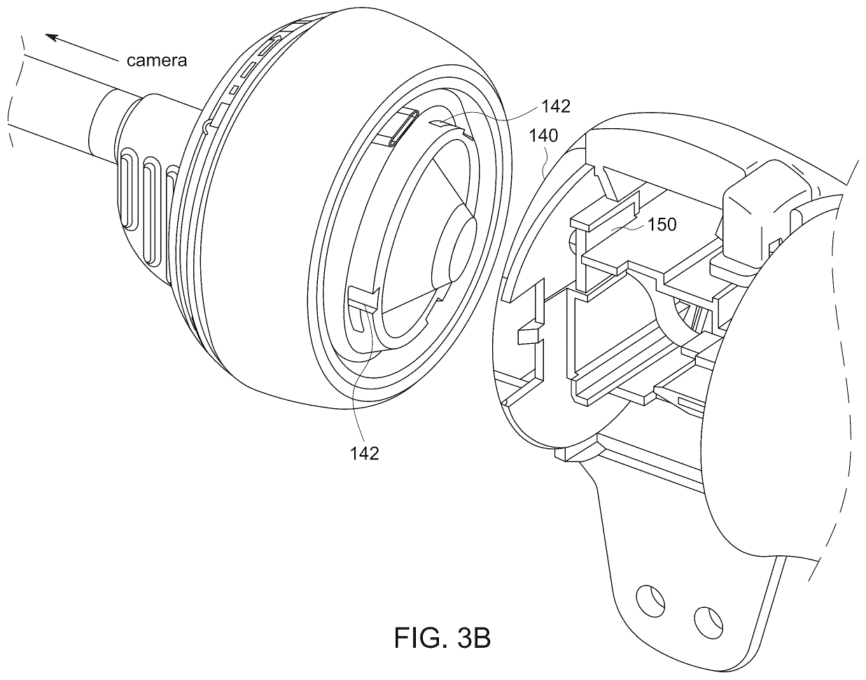

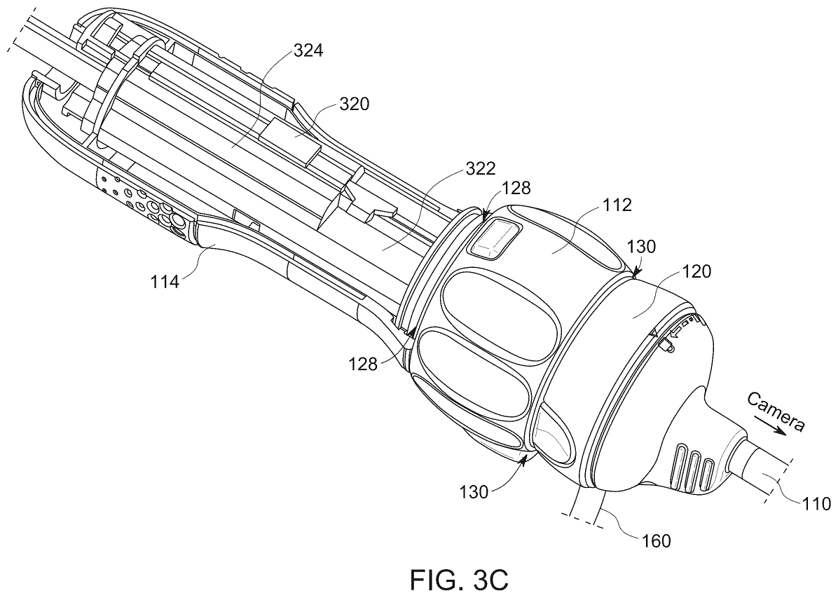

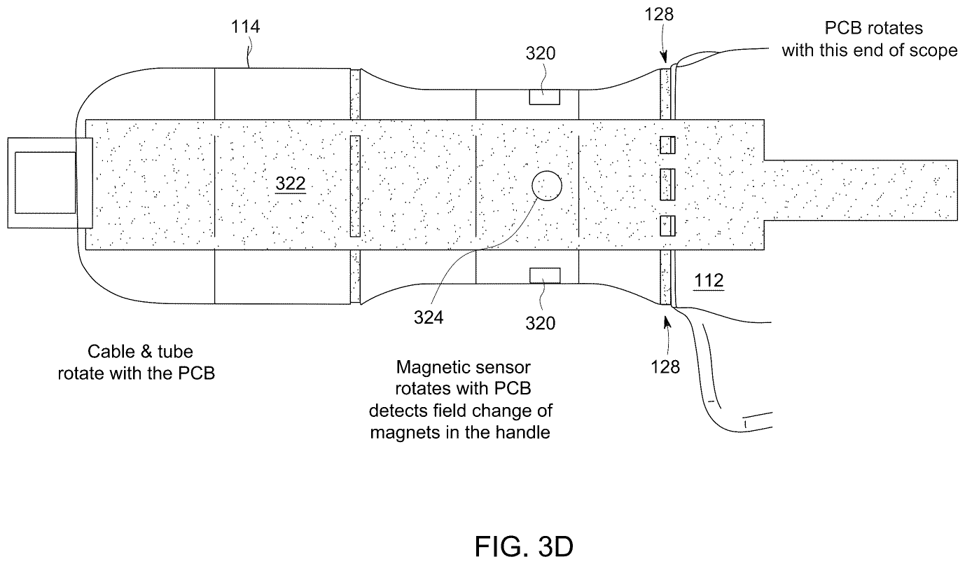

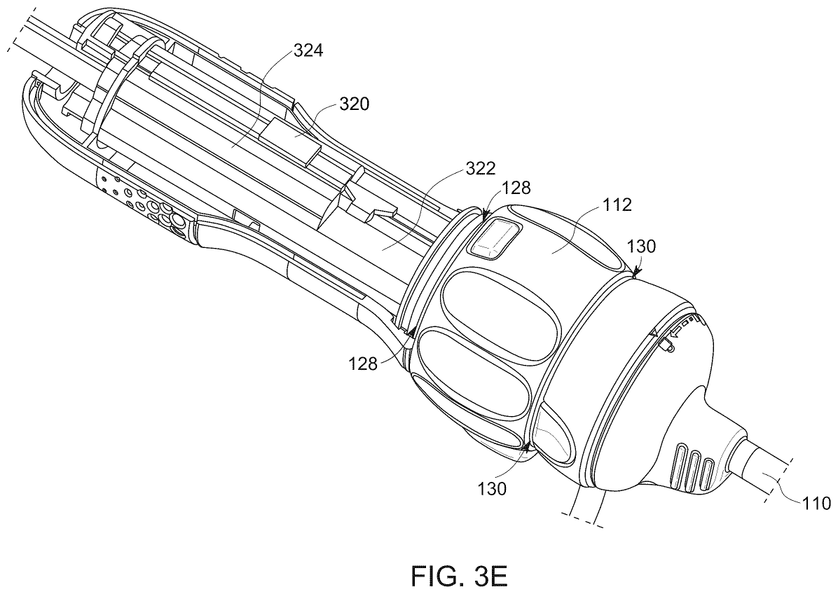

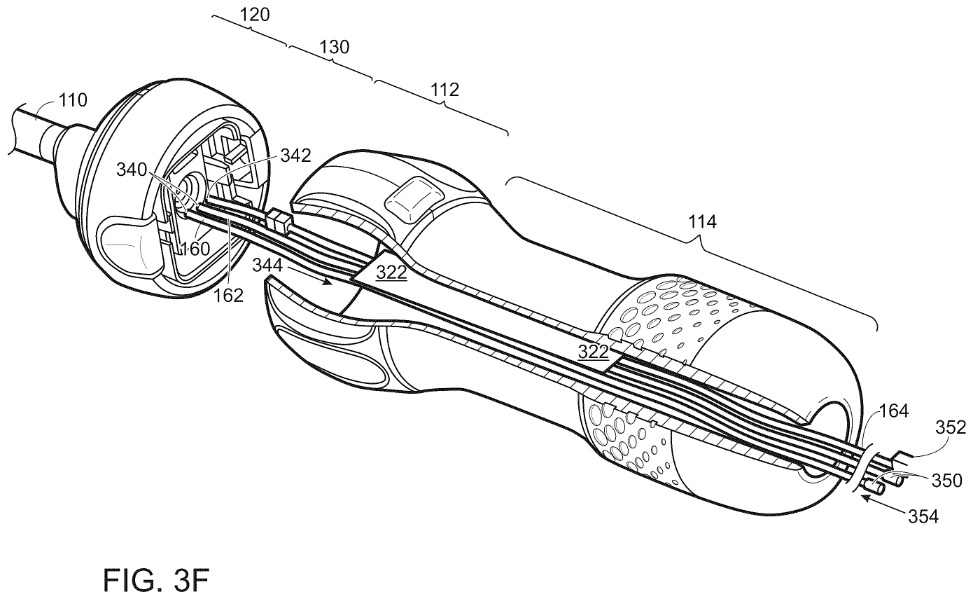

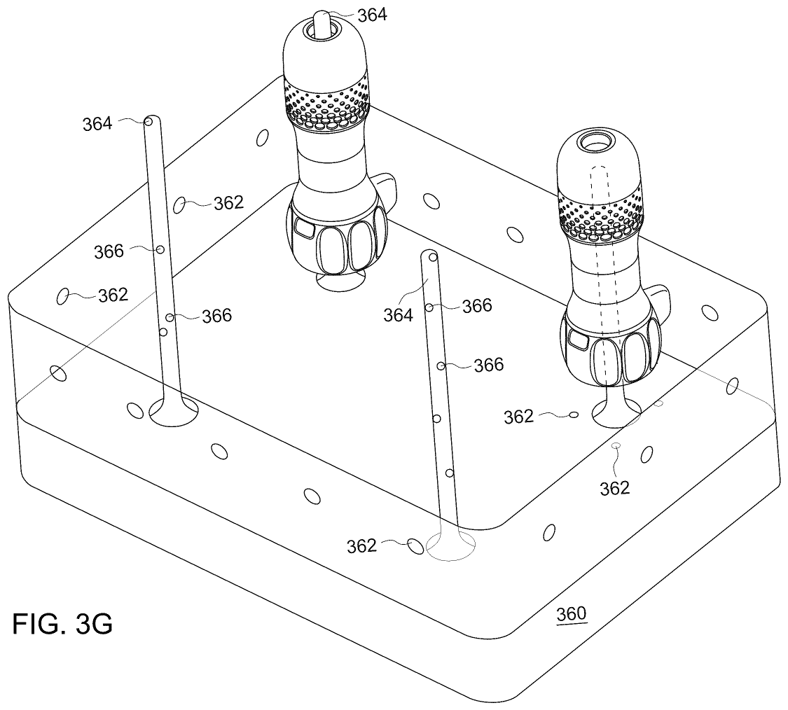

Referring to FIGS. 2A, 2B, 2C, 3A, and 3B, between the disposable cap 120 and rotation collar 112, three basic connections may be made:
A rotation-locking coupling 140, 142 to hold the disposable portion 120 to the reusable handle 112, 114. Coupling 140, 142 may have sufficient strength to transmit insertion and withdrawal forces, roll, pitch, and yaw torques, lateral forces, and similar forces from the proximal reusable handle 112, 114 to the distal disposable portion 120 and shaft 100, thereby to allow a physician to aim the illumination and/or camera as needed. Joint 130 between disposable cap 120 and rotation collar 112 may lie generally toward the distal end of the handle. The disposable cap and rotation collar 112 may engage through flat force-transmittal surfaces 144 at the center of joint 130 and around the circumferences, so that these forces are supported around the circumference of separable joint 130. One or more release buttons 146 may be pressed or squeezed to cause one or more locking snaps 148 to disengage. The mechanical connection may include a rotatable locking ring or other release/fixation mechanisms.
An electrical connection to supply power to the illumination source and camera 410, and to carry optical signals back from camera 410 to the processing board in handle 112, 114 and display system outside the endoscope. The disconnectable electrical connections for power and signal may be effected by a USB-A connector, USB-C connector 150, 152, mini HDMI connector, or similar connector that can maintain signal integrity for high speed signals. If illumination is conveyed by optical fiber 430, joint 130 may include an optical connector.
A disconnectable connection to any panning mechanism for camera 410 may be effected by a physical connector, such as a linkage.

In some cases, the camera/image sensor 410, LED 418, and electronic connections (and any mechanical connections for panning the camera/image sensor 410) may be removable from insertion shaft 110. Shaft 110 and cap 120 may be smooth and simple enough in shape to allow easy sterilization. Similarly, once the electronics are removed from interior of shaft 110, they may be sterilizeable as well. it may be cost-effective, especially in lower-labor-cost markets, to disassemble, sterilize, and reassemble the shaft and its interior components for reuse.

One or more fluid hoses 160 for irrigation liquid or inflation gas (or two hoses, one for fluid and one for gas) may enter through disposable cap 120, so that the entire set of fluid tubing for the irrigation/inflation channel may be disposable with the disposable shaft portion. In other cases (e.g., FIGS. 3E and 3F), a fluid hose 162 may enter the proximal end of the scope, and disconnectable fluid connections within joint 130 for fluid inflow and outflow may be effected by gaskets, O rings, and the like. Alternatively, connectors for the hoses may be outboard of the endoscope itself, either near the endoscope (for applications where it may be desirable to allow “quick change” replacement of the insertion shaft 110 in the course of a single procedure), or far from the endoscope, typically at the receptacle for waste fluid, to ease disposal of all hoses that are potentially contaminated by contact with the patient.

Disposable shaft 110, 120 may be designed to facilitate disposability of components that come into contact with bodily fluids. Because re-sterilization is often imperfect, patient safety may be improved by disposing of components that have come into contact with patient bodily fluids. To improve sterilizability, it may desirable to reduce componentry in the disposable component 110, 120 so that cost of the disposable component may be reduced, and to reduce surface features and crevices that may be difficult to sterilize. Thus, lens 460, image sensor, filter, LED 418, panning mechanism, and shaft 110 may be disposable. In addition, because shaft 110 is used for fluid inflow and outflow, and is disposable, sealing against bodily fluids may be unnecessary.

Referring to FIG. Referring to FIG. 2D, the replaceable/disposable shaft 110 and its mounting componentry 120 may be specialized to different types of surgery. For example, a purely diagnostic scope 172 may have an outer diameter of 1 to 3 mm. A replaceable disposable cap/shaft unit 110, 120, 178 for laparoscopic thoracic surgery may have a shaft of 400 mm length and diameter of 10 mm. Replaceable components 176 for arthroscopic surgery of knees and hips may be 155 mm in length, and 5.5 mm or 4 mm in diameter. For small joints, a replaceable shaft 174 of 2.9 mm diameter or less may be preferred. A replaceable shaft/scope unit with an bendable end 200 may be dimensioned for laparoscopic surgery. Typical dimensions for various surgical specialties may be as follows (measured in millimeters):

| | | Cannula diameter | | Scope diameter | |
|---|---|---|---|---|---|
| Scope Type | Discipline | Min | Max | Min | Max |
| Arthroscope (small | Arthroscopy | 2.8 | 4.0 | 1.9 | 2.9 |
| joint) | | | | | |
| Arthroscope (large | Arthroscopy | 4.7 | 6.0 | 2.9 | 5.3 |
| joint) | | | | | |
| Cystoscope | Cytoscopy | | | 2.9 | 5.3 |
| Encephaloscope | ENT | | | 2.0 | 4.0 |
| Hysteroscope | Gynecology | 3.7 | 7.0 | 2.0 | 5.0 |
| Laparoscope | Laparoscopy | | | 2.0 | 10.0 |
| Sinuscope | ENT | | | 2.0 | 4.0 |
| Thoracoscope | Pulmonary | | | | 10 |

Various single-use or replaceable components 110 may have different instruments at tip 116. For example, various single-use or replaceable shafts may have cameras 410 oriented at 0° (directly on-axis), 30°, 45°, 70°, and 90°.

Referring to FIG. 2B, disposable shaft portion 110, 120 may in turn be separable into an outer cannula 132 for protection and strength, and an inner shaft portion 134 carrying various illumination, optical, and fluid-carrying componentry. The scope may be sold as a handle unit 112, 114 with a set of ten or twelve or twenty replaceable shaft/cap unit 110, 120.

U.S. application Ser. No. 16/434,766, filed Jun. 7, 2019, and its formal drawings filed Aug. 13, 2019, are incorporated by reference.

### III. Extendable, Bendable, or Articulated Camera Tip

Referring to FIGS. 2F and 2G, the camera tip 202 may be slideable within cannula shaft 132, to be extendable and retractable. When extended, the distal portion of camera stalk 202 may be bendable, for example, as rigid segments 210 joined at articulation joints. In FIGS. 2F and 2G, cone 204 shows the field of view of camera 410. The extendable/bendable portion of shaft 202 may be formed of a series of elements that are each essentially rigid in the longitudinal dimension, but articulated at each joint to permit bending or flexure. The articulation may all be in the same dimension, as shown in FIGS. 2F and 2G. Alternatively, as shown in FIGS. 2H and 2I, articulation pivots 212 may be at alternating 90° angles, so that the bending may be in two dimensions, which in combination, may yield 360° of bending angles. Alternatively, the bendable portion 202 may be formed of elastic material, with an internal stiffener that is relatively stiff and incompressible against longitudinal compression dimension, and flexible in lateral and/or inclination bending. Bendable portion 202 may include two or four cable channels spaced around an outer surface, so that tension cables may cause bending in a desired direction.

Referring to FIGS. 2H, 2I, 2J, 2K, 2L, and 2M, in some cases camera 410 may be pannable within endoscope tip 400. For example, camera 410 and its illumination LED 418 may be mounted on one side of a substrate 220 formed as three rigid segments with two hinges 222, so that the two outer segments 224 may be moved relative to each other 226, and the center segment rotates in place, in the manner of a flexing parallelogram. The two outer segments 224 may be mounted in slide channels, and connected by cables 228 to controls at the handle. Referring to FIG. 2J, substrate 220 may be molded onto a flat flexible backing. The backing may contain folds 222 to create hinge points that allow the backing to fold into its parallelogram configuration (FIGS. 2K and 2L). Pivot points 230 may be molded into substrate 220. Referring to FIG. 2K, a flex circuit 232 may be laminated onto the substrate, and control tension cables 228 attached to the two ends. Camera (image sensor, lens, and filter) 410, illumination LED 418, pressure sensor, temperature sensor, or other sensors may be affixed to substrate 220. Referring to FIG. 2L, substrate 220 may be folded into three sides of a parallelogram, and a fourth side may be formed by a linkage connected to hinge points. Further details are described in U.S. Pat. No. 9,375,139, incorporated herein by reference.

Longitudinal movement 226 of one face of the substrate relative to the other changes the angle of the center segment, and thus the angle of the image sensor or other camera, and any other sensor. This may provide an adjustable view angle over a range that may be as large as 90°. The endoscope can also accommodate for a 180° or retrograde view where the endoscope has a flat top construction and a rotatable or living hinge rectangular endoscope architecture.

Passages and apertures for ingress and egress of irrigation, inflation, or other fluids may be provided in the tip. An aperture for irrigation fluid may be aimed to clear fouling from a window or lens over camera 410.

At least one of surfaces 224 may contain a metal strip bonded onto or into segment 224. The metal strip may be a spring steel or nickel-titanium alloy with a preformed radius of curvature. The metal alloy may alternatively be a malleable metal such as aluminum or may be a nickel-titanium (nitinol) alloy with a shape memory feature. The metal strip allows the elongated core to reliably bend in one plane of curvature. Where the memory substrate is spring-steel or nitinol, it may bend to a shape if malleable, or may be made steerable with a nitinol shape-memory component.

Referring to FIGS. 2N and 2O, lever 240 may be moved to advance/project or retract/withdraw camera 410 within insertion shaft (FIGS. 2F and 2G). Another switch/lever 242 (for example a paddle-shaped switch) may control cables or levers that flex the articulable tip by exerting tension on cables 228 that extend to the tip, to cause rotational articulation at joints 212 along the extendable portion 202 of shaft 110, thereby to control articulation of the camera tip to move in positive-y and negative-y directions. Another lever may be used to control camera panning (FIGS. 2H, 2I, 2J, 2K, 2L, and 2M).

Referring to FIGS. 2P and 2Q, a four-point control 244 may control four control cables 228 or rods or other load bearing components to the articulated or bendable portion of the extendable/retractable and/or articulated camera shaft 202, so that the camera tip may be articulated in positive-x, negative-x, positive-y, and negative-y directions.

Referring again to FIGS. 2D and 2E, the extendable/retractable and/or articulated camera shaft 110, 200 may be used with a reusable handle, disposable tip configuration. The extendable/retractable and/or articulated camera shaft may be used with a reusable or single-use unibody scope configuration.

The articulated camera tip 200 may be especially useful in abdominal thoracic laparoscopy. Typically, during abdominal surgery, the abdominal cavity is inflated with carbon dioxide, to give the surgeon a large open field of view. This gives an extendable/retractable and/or articulated tip space to move. The extendable/retractable and/or articulated tip may be useful to provide a view behind an organ, such as the stomach or liver. If the surgeon only has a fixed view endoscope/laparoscope, the only way to obtain a view behind an organ would be to open another port from the opposite side of the body.

### IV. Additional Features of an Endoscope

Referring to FIGS. 2A and 2B, disposable shaft portion 110, 120 may in turn be separable into an outer cannula 132 for protection and strength, and an inner shaft portion 134 carrying various illumination, optical, and fluid-carrying componentry. Illumination may be provided by LED 418 at or near the distal tip, or via fiber optics 430 from an illumination source in the handle, or illumination at an external controller.

Referring again to FIG. 2A, the endoscope may have a handle 112, 114, 120, and a shaft 110 for insertion into a body. At or near distal tip 116 of the shaft 110 may be a lens, electronic image sensor, filter, or other optical component 410. The camera's orientation may be fixed in the scope, or may be pannable. Camera 410 may be at tip 116, looking out from the shaft, or may be recessed a short distance behind the structural tip of the shaft. Also at or near the tip may be an illumination source, such as LED 418. Tip 116 may have a rigid pointed tocar tip, or may have a spoon-shaped portion that reaches past the distal surface of the window in tip 116, or may be flexible (in the manner of the tip of a colonoscope), in each case extending a little beyond the distal surface of the window in tip 116 to provide physical protection to the tip 410 during insertion or to protect the camera 410 from a surgical cutting device.

Illumination may be in visible light, infrared, and/or ultraviolet. In some cases, an illumination LED (light emitting diode) or other illumination source may be placed in reusable handle 112, 114 or in a docking station/controller, and the disposable shaft may have fiber optics 430 to transmit light to the tip, and joint 130 may have an optical coupler. In other cases, illumination LED 418 may be placed in tip 116 to illuminate the surgical cavity directly; in such cases, joint 130 may have a power connector. In some cases, LED 418 may be recessed from the tip, or placed somewhere in the shaft, or may be in an external controller, and optical fiber 430 may carry illumination light to the tip. Optical fiber 430 may be configured, for example, with a split, so that light will be arrayed in a desired pattern around the image sensor to better distribute the light into the surgical cavity around the camera.

The shaft 110 itself may be rigid, made of a nonbioreactive metal such as stainless steel or coated aluminum. In some cases, a surgical cavity around endoscope tip 400 may be insufflated by gas (typically carbon dioxide), or irrigated by saline solution. In either case, fluid inflow and outflow may be effected by channels through the shaft.

Shaft 110 may also carry power wires to illumination LED 418 and camera 410, and carry signal wires that carry a video signal back from camera 410 to electronics in the reusable portion 112, 114 of the handle. Electrical power to camera 410 may be supplied over conductors in a flexible cable or on a printed circuit board (flexible or rigid), and may be insulated with a conformal and insulating coating such as parylene. This same flexible circuit board 416 may have signal conductors for the video signal from image sensor 410. The video signal may be transmitted from image sensor 410 to the handle using any video signal protocol, for example, MIPI-CSI2 (Mobile Industry Processor Interface-Camera Serial Interface2) or HDMI. In some cases, a parylene coating may improve biocompatibility.

Shaft 110 may also carry cables or other mechanical elements to control panning of camera 410.

Referring to FIGS. 3A, 3C, and 3E, rotation collar may have various features that make rotation easy. For example, depressions 302 may provide a good grip for fingers for light roll torque. Fin 304 may provide greater leverage for greater roll torque, and may also provide a fixed rotational point of reference.

A button 310 may perform various functions, such as turning illumination LED 418 or fiber optic illumination drivers on or off, taking pictures, starting and stopping video, and the like. A single button may perform all these functions based on the nature of the press. For example, press-and-hold for 3 seconds may turn the illumination on and off. A quick press may capture a single-frame still picture. A double-click may start and stop video recording. The push button may have a magnet at the bottom of the button, with a Hall effect sensor on the handle board. This may provide a button with no physical contact that can fail due to infiltration by liquid or biological material.

If camera 410 at the tip 116 of shaft 110 is pannable or has other controllable features, there may be a control (for example, a lever, or a touch-slide panel, etc.) near button 310 to control that adjustment of camera 410.

One or more ultraviolet LEDs or other illumination source may be placed inside handle 112, 114, inside shaft 110, or near tip 116 to assist with insuring sterility of the internal components of the device or of the water as it passes thru the device

Referring to FIGS. 3C, 3E, and 3F, irrigation/insufflation hose(s) 160, 162 may enter at various points through the handle. For example (FIG. 3C), irrigation/insufflation hose(s) 160, 162 may enter laterally, somewhere near the distal end of the handle, for example, through fin 304. Or, as shown in 3E and 3F, irrigation/insufflation fluid/gas hose(s) 160, 162 may enter through the proximal end of handle 114. This hose may then be disconnectable via a fluid disconnect joint 320 within joint 130. In cases where hose(s) 160 for insufflation fluid/gas enters through disposable cap 120 (FIG. 3C), various joints and strain relief features 340 may be used to hold hose(s) 160 in place.

Referring to FIG. 3B and FIG. 3F, electrical connectors 150, 152 such as USB-A, USB-C, or mini-HDMI connectors may be used to connect camera 410 to a circuit board interior to handle 114.

Referring to FIG. 3B, rotation-locking coupling 140, 142 may lock disposable cap 120 in rotational relationship to rotation collar 112. Various rigid and resilient features 144, 148 may lock them together for other forces and torques, and release buttons 146 may permit them to disengage to allow replacement of disposable cap 120. The coupling between cap portion 120 and rotation-locking coupling 140, 142 may place much of the stress at the periphery of the joint, so that joint 130 may carry and transmit forces (especially torques) well.

Referring to FIGS. 2A, 3C, and 3D, rotation between the handle's stationary portion 114 and rotation collar 112 may be provided via a rotational bearing at joint 128.

Referring to FIGS. 3C, 3D, and 3E, proximal handle 114 may contain a number of components, typically components that have only incidental patient contact (and therefore present less risk of cross-infection), are higher in cost (and therefore desirably reusable), and either sterilizeable or may be covered by a sterility sleeve. For example, proximal handle 114 may hold power transformers or power supplies, signal amplifiers, one or more buttons or other controls (either electromechanical, servoelectro, or mechanical) for illumination LED 418, fiber optics, and/or camera, for panning camera 410, controlling illumination and camera view, rotation sensors for righting of an image from camera 410, and the like. The handle may also include connections to external sources and destinations of power, signal, fluid, and the like.

Proximal handle 114 may include rotational sensors so that an angular orientation of camera 410 may be ascertained. For example, the inner surface of proximal handle 114 may mount one or more magnets 320, and printed circuit board 322 (which rotates with rotation collar 112 and disposable cap 120) may have Hall effect sensors 324 that detect the magnets. This may be used to compute a rotational orientation, which may in turn be used to “right” the image from camera 410 on a video display screen.

The distal tip of the shaft, camera 410 mounted therein, and the mounting of componentry within shaft 110 may be designed to be robust. Occasionally, during surgery, the tip of the endoscope may come into contact with a shaver, ablation probe, or cauterization probe, and it may be desirable to have the tip be robust to such contacts. To reduce risk that componentry may be dislodged and left in the patient, the disposable shaft and its componentry may be designed to avoid joints that are at high risk of mechanical failure. A disposable optical system may prevent the image degradation that occurs when nondisposable optics are reused in multiple surgical procedures.

Endoscopes as a genus include arthroscopes, laparoscopes, colonoscopes, and other specialized scopes for various body cavities. For an arthroscope for joint surgery, the shaft may be as small as 6 mm, 5 mm, 4.5 mm, 4 mm, 3.6 mm, 3.3 mm, 3 mm, 2.8 mm, 2.6 mm, 2.4 mm, 2.2 mm, 2 mm, or 1.8 mm, and highly rigid. For other endoscopes, such as a colonoscope, the diameter may be larger, and the shaft may be flexible.

The endoscope may be delivered as a handle and multiple tips, each tip individually sealed for sterility.

Referring to FIG. 3F, hoses 160, 162 for irrigation/insufflation fluid/gas in, irrigation/insufflation fluid/gas out, and electrical connection cord 164 may be permanently affixed 340, 342 to disposable cap 120. This arrangement may allow that hose 162 that carries water out of the surgical cavity, and which is therefore contaminated, may be disposable, and no fluid will come into contact with the reusable part 114 of the handle. Hoses and cord 160, 162 may be routed through channel 354 running the length of reusable handle 112, 114. Channel 344 may be of inner diameter large enough to permit easy passage of hoses and cord 160, 162, 164, and connectors 350, 352, and have a continuous smooth wall that permits easy sterilization, to permit ready replacement of the replaceable components. Channel 354 may be off the central axis, to allow printed circuit board 322 to lie on the central axis. Connectors 350, 352 at the end of hoses and cords 160, 162 may be small enough to pass through channel 354. Thus, replacement of shaft 110, cap 120, hoses and cords 160, 162 may be effected by threading connectors 350, 352 and hoses and cord 160, 162 through channel 344. Electrical cord 164 may have a connector 354 at or near joint 130, and hose(s) 160 for irrigation/insufflation fluid/gas flowing into the surgical cavity may likewise have a connector at joint 130 to allow this hose(s) to be reusable, or may be permanently affixed 340 to reduce possibility of leaking. Having hoses and cable 160, 162 roughly on-axis reduces undesirable cable flop as the scope is in use, and reduces undesirable torque on cap 120. Forming shaft 120, cap 120, and hoses 160, 162 as an integral unit for replacement reduces possibility of leaking, and improves sterility of the replacement operation.

Referring to FIG. 3G, reusable handles 112, 114 may be sterilized in a sterilizer 360. Preferably, hose(s) 160, 162 and all other portions of endoscope 100 that come into contact with the patient, or with fluids that have come into contact with the patient, are disposable, and the design for reusable portions 112, 114 may reduce contamination by avoiding contact with the patient's bodily fluids. Sterilizer 360 may be arranged to accept one or more reusable handles 112, 114, and irradiate them with ultraviolet light from ultraviolet LEDs 362. Rods 364 that pass through handle channel 344 may have ultraviolet LEDs 366 arranged along their lengths, to sterilize internal channels 344.

### V. Endoscope Tip

Components of endoscope tip 400 may be designed to permit camera/image sensor 410, lens, filter, an illumination emission source, and a window to be mounted within a confined space, such as an endoscope or an arthroscope for joint surgery, having a diameter of 6 mm or less, 5.5 mm or less, 5 mm or less, 4.5 mm or less, or 4 mm diameter or less. In some cases, fluid management may be managed in the same space. In some cases, the shaft may have the strength and rigidity commonly found in arthroscopes. In some cases, the illumination emission may be by one or more LEDs 418 located at or near the endoscope tip. In other cases, the illumination emission may be via fiber optical fibers 430 and/or light guides 450 that conduct illumination light around the camera/image sensor 410, within the diameter of shaft 110.

### V.A. Molding and Assembly of Components of the Endoscope Tip

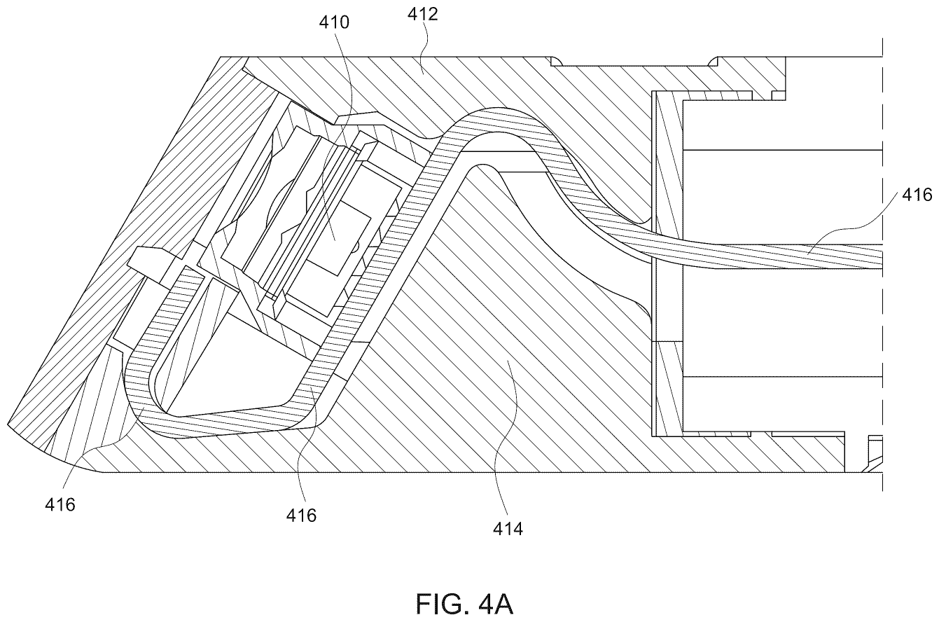

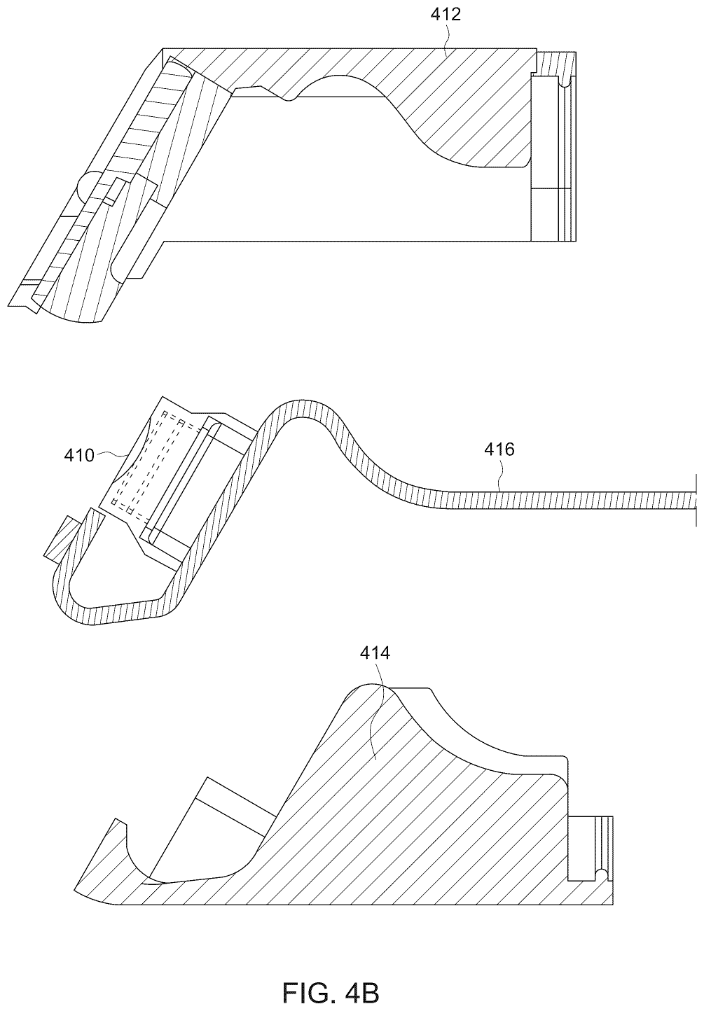

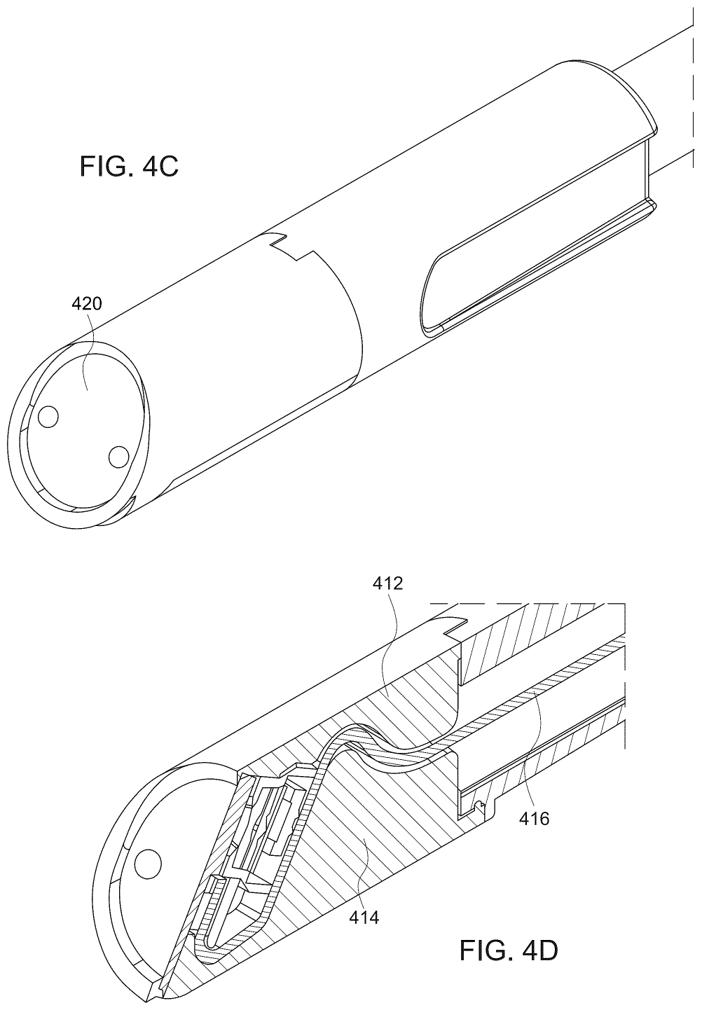

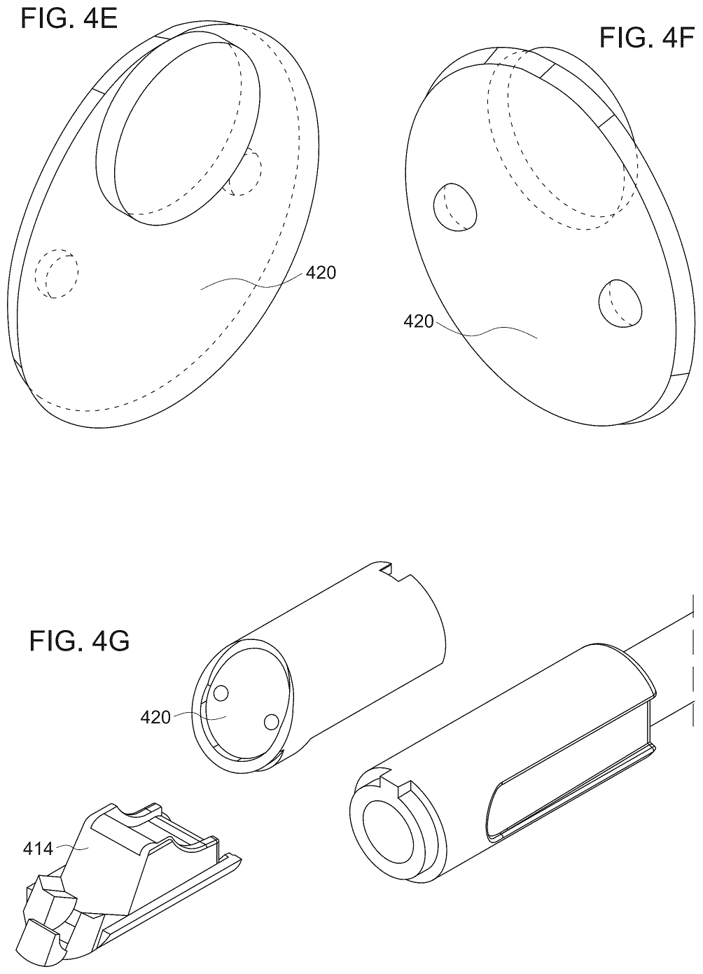

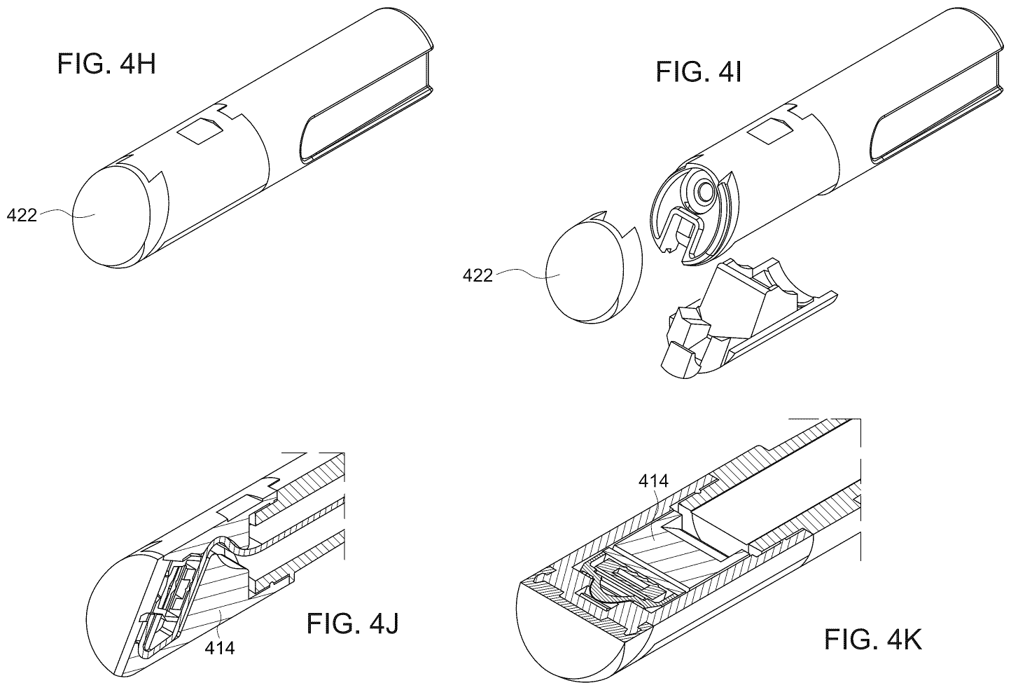

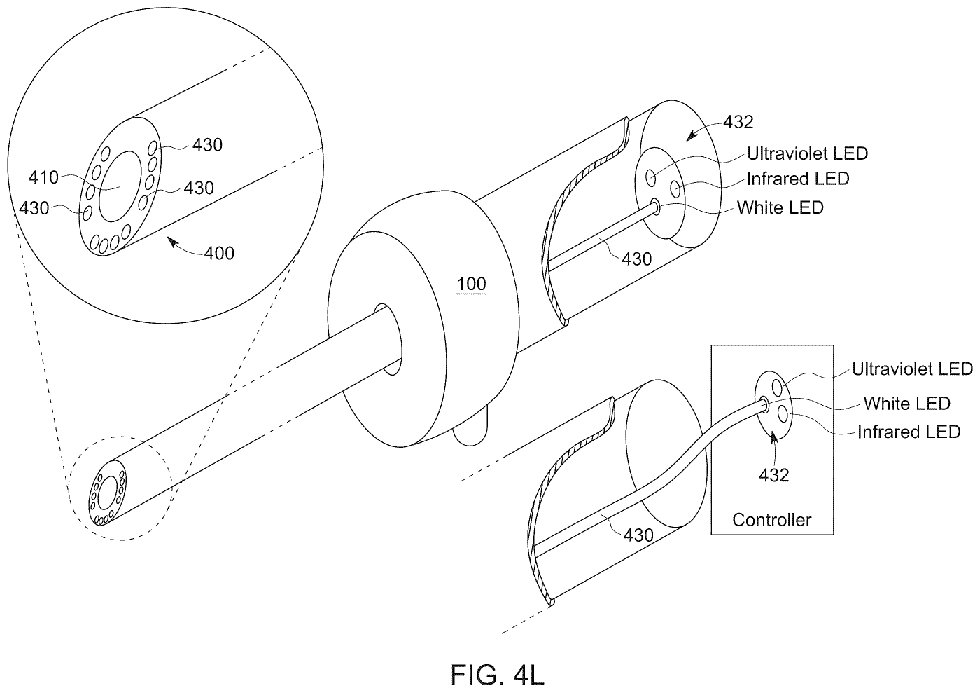

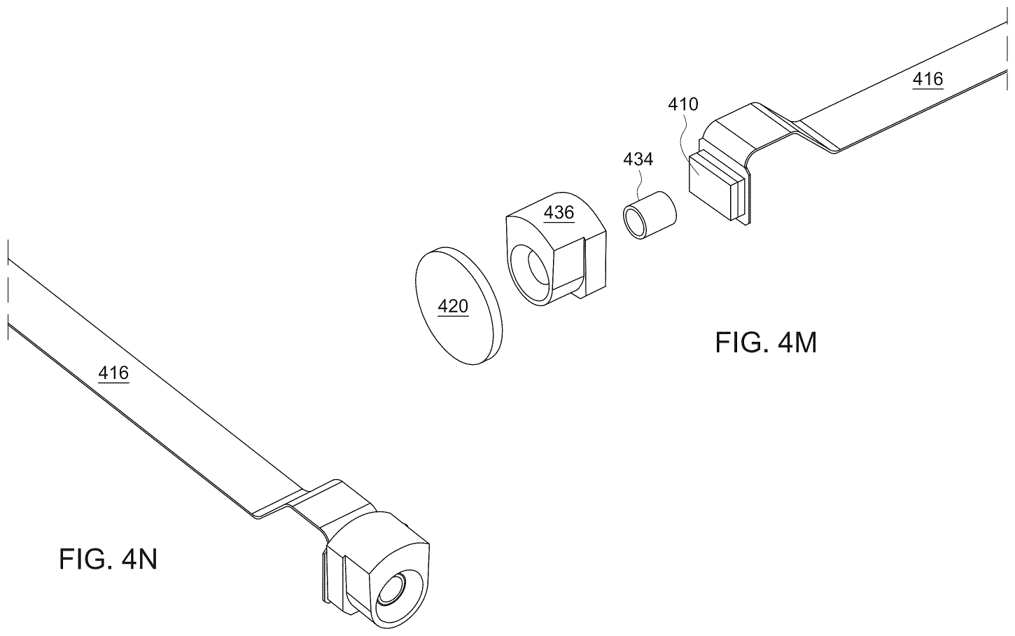

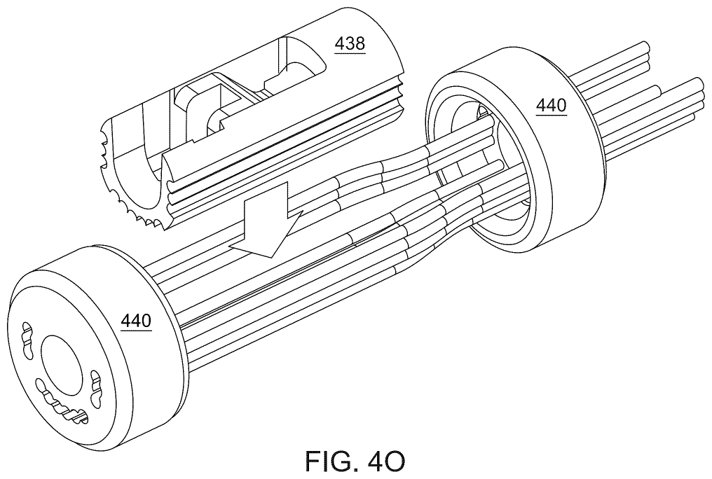

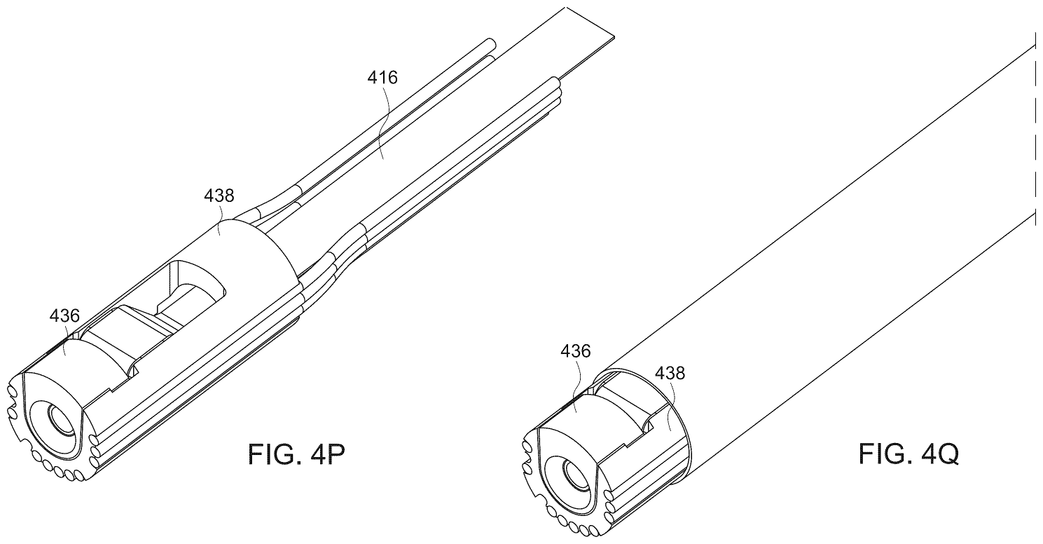

Referring to FIGS. 4A and 4B, endoscope tip 400 may be formed of top brace 412, bottom brace 414, and flexible circuit board 416. The structural components may be formed of an opaque biocompatible plastic such as Lustran 348. Camera/image sensor 410 may be mounted on one side of flexible circuit board 416, and an illumination LED 418 on the other side. Clear window 420 may protect camera/image sensor 410 from the outside environment, such as the tissues and bodily fluids of an endoscopic procedure, and pressurized insufflation fluids. The entire assembly may be locked together via overmolding, fusion welding, a plastic welded cap, biocompatible adhesive, or the like.

Referring to FIGS. 4A and 4B, to assemble the components, one end of flexible circuit board 416, the end with LED 418 mounted thereon, may be slotted into a slot or channel in top brace part 412, which holds LED 418 into place. Then board 416 may be folded around a bend in top brace 412, so that camera 410 comes into place through its hole in top brace 412. The folding and rotation brings LED 418 close to camera/image sensor 410, which permits the assembly to fit within the 5 mm diameter of tip 400. Then bottom brace 414 may be brought into place, which holds top brace 412, bottom brace 414, circuit board 416, LED 418, and camera 410 in their positions of mutual alignment. A locking notch and clip, or ultrasonic welding may hold this assembly together for a time. Then an overmolding or similar step may lock everything together.

Referring to FIGS. 4C, 4D, 4E, 4F, and 4G, transparent window 420 may cover camera 410 to protect it. Window 420 may be two thicknesses, a thicker region over camera 410, and a thinner region for the portions used for mounting and for the illumination emitter (LED, end of fiber optic fibers or light pipe, etc.) to shine through. The window may have embedded features such as grooves, inserts, or other opaque material isolating the light path out of the window from the imaging path of the illumination reflected off of the object of interest into the window. Alternatively, two separate windows may be utilized to isolate the illumination light path out of the window from the imaging path of the illumination reflected off of the object of interest into the window, one over the camera and one over the illumination. A peripheral ridge of endoscope tip 400 may extend beyond window 420 by a small amount. Top brace 412 may include an opaque wall that surrounds LED 418, fiber optic fibers, or light pipe. These shapes, alone or in combination, may offer one or more of the following advantages. First, these shapes may reduce stray light from LED 418 (or other illumination) being internally reflected into image sensor 410. Second, the thickness of the window 420 on the lens side may reduce vignetting artifacts, when the edge of the field of view of a image sensor is occluded or lens 460 gathers less light toward its edge. Likewise, the shape of the lens may be used to reduce distortions such as fisheye distortions. Third, the ridge may tend to keep tissues away from lens 460, reducing obscuring and improving view. Alternatively, a window may be placed only over the camera element and the illumination emitter may have a separate window or the light emitter is able to protrude through an opaque holder to the same plane as the outer surface of the camera window, sealed to the opaque light emitter holder with an adhesive.

Window 420 of FIGS. 4E and 4F may be placed over the surface of the assembly of circuit board 416 with LED 418 and camera 410, top brace 412, bottom brace 414, and window 420 (FIGS. 4B, 4C). Then the assembly with window 420 may be locked together via overmolding of a covering sheath (FIGS. 4C, 4D, 4G). This overmolding may provide watertightness to the entire tip assembly. The overmolded assembly may then be fitted onto the tip of the endoscope's insertion shift. A plastic window may be lower cost than glass, which reduces cost, enabling the scope to be disposable after one-time use. The plastic may be exceptionally high index of refraction, above 1.5, with high clarity and high moldability. The co-molded clear plastic window may be over molded over the opaque structural parts. The window may be applied in a two-shot mold, in which the opaque structural components (the brace/chassis 412, 414, 438 are injected first at a high temperature and allowed to cool, and then window 420 may be injected at a lower temperature. Components of the brace/chassis, the lens and flex PCB may be ultrasonically welded, laser welded, fusion welded, or affixed via adhesive. This weld or adhesive may provide a watertight seal to prevent fluids from reaching the sensor and LED 418.

Referring to FIGS. 4H, 4I, 4J, and 4K, in other cases, in an alternative, clear window 422 may be overmolded onto top brace 412, before circuit board 416, LED 418, camera 410, and bottom brace 414 are assembled. As window 422 is overmolded, a flat platen may be placed to project through camera 410 hole to provide a mold surface, to provide an optically smooth back surface of window 422. The mold may be flat (planar), or may have a desired curvature to form a convex or concave lens in overmolded window 422. As shown in FIG. 4K, the circumferential edges of top brace 412 may be shaped to provide a secure lock that engages overmolded window 422. Then circuit board 416 with LED 418 may be inserted into the slot, and folded around top brace 412, and then bottom brace 414 may be snapped into place and ultrasonically, laser, or fusion welded.

Referring again to FIG. 4A, at its top right and bottom right corners, and referring to FIG. 4B, at the top right corner of top brace 412 and the lower right corner of bottom brace 414, energy directors molded into top brace 412 and bottom brace 414 may improve ultrasonic welding. Ultrasonic welding may improve adhesion and watertightness among top brace 412, bottom brace 414, and flow director components. The energy directors may be molded as triangular-profile ridges on the inside surface of the top-brace-bottom-brace assembly. The inner diameter of the energy directors may be slightly smaller than the outer diameter of the flow director, so that ultrasonic welding causes the energy directors to melt and to form a seal.

Taken together, these features may provide an endoscope tip 400 of very small diameter, such as 4 mm or less, 5 mm or less, 4.5 mm or less, or 4 mm or less, 3.6 mm or less, 3.3 mm or less, 3 mm or less, 2.8 mm or less, 2.6 mm or less, 2.4 mm or less, 2.2 mm or less, 2 mm or less, 1.8 mm or less, or a tip 400 slightly larger than an endoscope shaft, with all components fitting inside that tip diameter. Mounting LED 418 and camera 410 on opposite sides of flexible circuit board 416 may assist in making the entire assembly more easily manufacturable. That manufacturing may involve inserting the end of a flexible circuit board 416 into a slot, and wrapping board 416 around a molded part or wrapping board 416 into a channel between molded parts to place various components in their preferred operating orientations. This positioning of board 416, including bending and wrapping, may build some additional slack into the positioning of board 416, which may create some strain relief and improve reliability. Components may be ultrasonically welded together. Overmolding may be used to structurally hold components together and to provide a watertight seal. Overmolding of clear window 420, 422 over the structural components 412, 414, 438, or the structural components molded onto a clear window, may likewise contribute to a watertight seal.

This overall design philosophy may permit reconfiguration and reuse of much of the engineering for endoscopes of varying size, scalable depending on how small the sensor is and the need of the specific surgery (in contrast, for rod-lens scopes, many design decisions are specific to a single design). Features that contribute to scalability include the use of a single flex board, the top and bottom brace or chassis 412, 414, 438, and overmolded window 420.

### V.B. Fiber Optic Illumination for a Single-Use Use Scope Tip

Referring to FIG. 4L, a single use endoscope 100 or single-use tip for a reusable handle may have image sensor 410 on the tip. Single use endoscope 100 may use fiber optical fibers 430 to deliver illumination light. Plastic optical fibers 430 may offer an attractive combination of attributes for disposable or single-use endoscopy applications, including cost, flexibility to bend around curves and for motion during a surgical procedure, numerical aperture (the cone of angles over which the fiber radiates light, and the cone within which it accepts), low heat radiation at the surgical site, and manufacturing resilience. Fiber optic illumination may deliver illumination adequate for applications such as laparoscopy, where the objective surface may be 200 or 300 mm from camera 410, while avoiding problems of heat dissipation that may arise by placing LED 418 at the tip. Fiber optic illumination may reduce complexity of chip on tip circuitry in the confined space of endoscope tip 400. Fiber optic illumination may permit use of multiple illumination sources of varying wavelength to be coupled at the collection end of the fiber, to change the illumination at endoscope tip 400.

Referring to FIG. 4L, one or more illumination sources 432 may be located either in the reusable endoscope handle or base station/IPU/Master Controller. Illumination source 432 may be one or more of single color LED, a white light source, a tricolor white LED, infrared or ultraviolet light, etc. Illumination source 432 may be LED 418, combination of LEDs, flash lamp, incandescent lamp, laser, or other illuminator. Fiber 430 may be coupled to illumination source 432 at the collector end by butt adjacency or other collection mechanisms. In some cases, where multiple illumination sources 432 are provided, they may be on a rotating carousel, sliding multiplexer, or other switch that brings successive ones of the multiple illumination sources into butt adjacent coupling with the coupling end of the light fibers 430. Light source devices 432 that are the same size or slightly larger than the collector end of optical fiber 430 offer the most efficient butt adjacency coupling.

Plastic light fibers 430 are available as fluoridated polymer optical fibers tradenamed Raytela™ from Toray Industries, Inc. of Japan, or other vendors of plastic optical fibers. Plastic light fibers 430 may reduce cost relative to glass fibers 430, which may be an especially important consideration in a single-use or disposable endoscope design. Plastic optical fibers 430 may be formed of two different plastic resins that have two different indices of refraction, the higher index resin used as a core, and the lower index resin used as a cladding layer. The boundary between the layers may provide total internal reflection to conduct light down the fiber 430. The diameter of fibers 430 may be chosen to optimize several simultaneous characteristics. The amount of light that can be carried per fiber is roughly proportional to cross-section area. The cost of optical fiber 430 is primarily proportional to length, with a smaller cost growth with diameter. Likewise, manufacturing cost generally grows with the number of fibers, and grows with the number of fibers that break or are damaged during manufacture, so fewer larger-diameter fibers tends to be lower cost. On the other hand, mounting camera 410 and any working channel apparatus is generally more difficult, and optical fibers 430 are easier to fit into a small space if they are smaller diameter, which tends to favor a larger number of smaller-diameter fibers 430. To optimize among these tradeoffs, in some cases, at least one fiber, at least two fibers, at least three fibers, at least four fibers, at least six fibers, at least eight fibers, at least nine fibers, at least twelve fibers, or at least 15 fibers may be used. The fibers may be about 0.4 mm, 0.5 mm, 0.6 mm, 0.75 mm, or about 1 mm in diameter. They may be placed around the periphery of the working tip 400 of scope 100. In other cases, especially with larger diameter scopes, fewer fibers of larger diameter may be used, or light fibers may feed into light guides 450 to conduct illumination around image sensor 410 in the region of tip 400.

Referring to FIG. 4L, in some cases, fibers 430 may be relatively uniformly spaced around the 3600 periphery of tip 400. Greater uniformity of the placement of the illumination fibers 430, and centering on camera 460, may reduce variability of illumination across the imaging field, shadows, and other undesirable artifacts. In other cases, fibers 430 or the distal face of light guides 450 may be distributed over some arc less than 360°, such as at least about 180°, at least about 240°, at least about 250°, at least about 260°, at least about 270°, or at least about 300°. In some cases, the endoscope may be used very close to the anatomical structures on which surgery is performed, so distributing the illumination emission around the periphery may reduce glare and hot spots. In some cases, larger fibers 430 may be used for part of the periphery, and smaller fibers 430 may be used for a part of the end of the endoscope that is crowded with other mechanical components. The closer the fibers 430 can approximate a uniform 3600 distribution, the more uniform the lighting, and thus the better the image. Using fibers 430 with a larger numerical aperture or other dispersion at the end may likewise improve dispersion, and thereby improve uniformity of illumination and image quality. Non-circular fibers 430 may be used to allow greater surface area of the illumination end of the fibers, and thereby provide better illumination.

Referring to FIGS. 4M and 4N, camera/image sensor 410 may be mounted on a flex circuit board 416. Lens and filter 434 may be held n place by an inner tip part 436, and these parts may be assembled into a lens subassembly 460.

Referring to FIG. 4O, two jigs 440 may have a cap form, with inner diameter matching the outer diameter of tip chassis 438, and with throughholes conforming to the position of channels on the outer surface of tip chassis 438, the channels providing circumferential positions for optical fibers 430. The holes in the two jigs are typically in pair-wise opposition to each other. Optical fibers 430 may be routed through the holes in the jig parts, from matching hole to matching hole. Referring to FIGS. 4O, and 4P, tip chassis 438 may have grooves along its periphery to capture fibers 430, and a set of inner channels and features to hold the camera/inner tip part assembly. Referring to FIGS. 4O and 4P, tip chassis 438 may be placed into the nest of fibers held in place by jigs 440. Referring to FIG. 4P, the camera/lens/filter/inner tip part assembly may be placed into the tip chassis 438, and flex board 416 laid into a channel formed by the optical fibers. Then rear jig 440 may be slid off the end of the flex board 416 and fibers 430.

Referring to FIG. 4Q, fibers 430 may be cut to match the surface of tip chassis 438. The ends of the fibers may be polished, or the ends may be treated to diffuse light, as discussed below in section V.D. The ends of the fibers may be covered with window 420, typically glass or sapphire. The outer/distal surface of window 420 may be coated to accept a coating, such as that described in U.S. application Ser. No. 16/069,220 and U.S. Provisional App. Ser. No. 63/193,387, incorporated by reference. Window 420 may have a coating to reduce lighting artifacts, such as reflection. Window 420, 422 may be held in place by an adhesive. This adhesive may fill any air gap between the end of optical fibers 430 and window 420. Removing the air gap may improve light transmission from fibers 430 through the window 420. In some cases, a lens, filter, or window 420 may be overmolded over the assembly shown in FIGS. 4P and 4Q. The assembly of the fibers 430, tip chassis 438, inner tip part 436, camera 410, and flex board 416, and window 420 may be threaded through the stainless steel tube of the scope's insertion shaft, and slid into place for either friction fit, or to be held in place by an adhesive, such as epoxy or an optical grade UV-curing adhesive (available form Norland Products Inc. of Cranbury NJ, from the Henkel Adhesives division of Loctite, from Dymax Corp. of Torrington CT, and others), or to be held in place by a detent or deformation of the tube. Adhesive may also serve to seal the tip against intrusion by fluids. The surface of camera 410 may be recessed into the stainless steel tube. The surface of window 420 may be flush with the end of the tube, or may be recessed slightly, to provide protection against scratching or dislodgement of the window. The recess may be covered by a clear lens, either entirely flat, or with features or lenses analogous to those shown in FIGS. 4E and 4F.

V.C. A Tip Design with Light Guides

Light fibers 430 may be extruded in shapes that improve light delivery, such as rectangular, or a U-shaped light guide 450 that would extend the length of the endoscope from the illumination source to the U-shaped emission surface at the distal end of the endoscope. In some cases, individual fibers 430 may be replaced, for at least some portion of their length, by a shaped light guide 450, such as a circular or U-shaped ring of clear light guide around the periphery of the tip chassis 438, 480. Light guide 450 may be a two-component structure, with two different indices of refraction for internal reflection analogous to optical fiber. In other cases, light guide 450 may be formed of a clear light transmission medium coated by a reflective coating, such as aluminum, gold, or silver. In some cases, the shaped light guide 450 may extend only a short distance, for example, the length of the inner tip part, and traditional circular fibers may be used to bring light from the illumination source to the proximal end of light guide 450 at the tip chassis 438, 480.

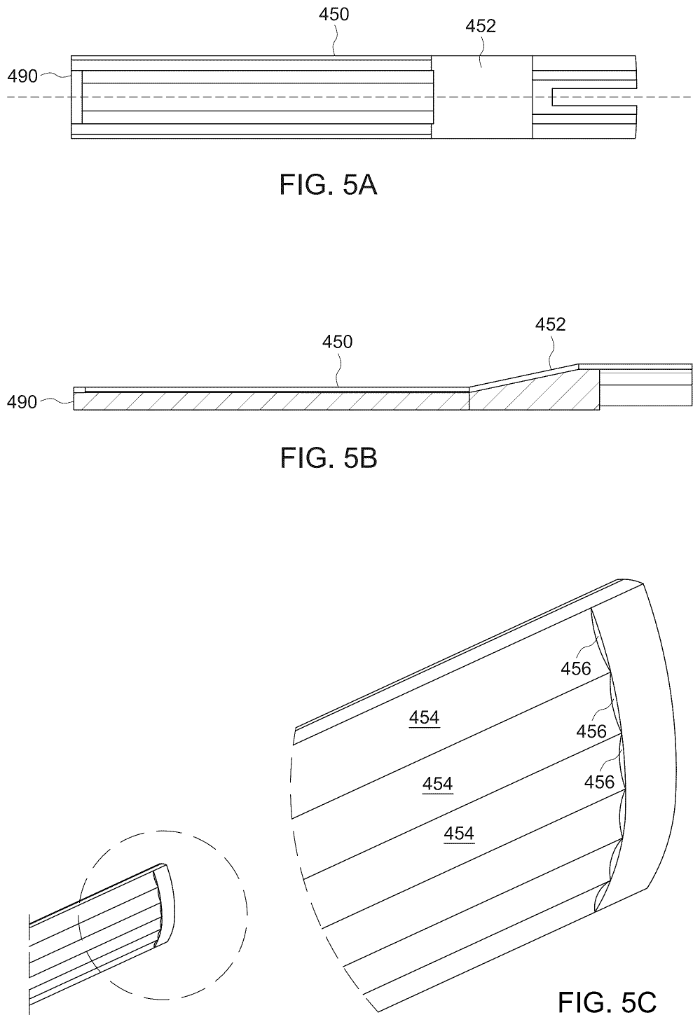

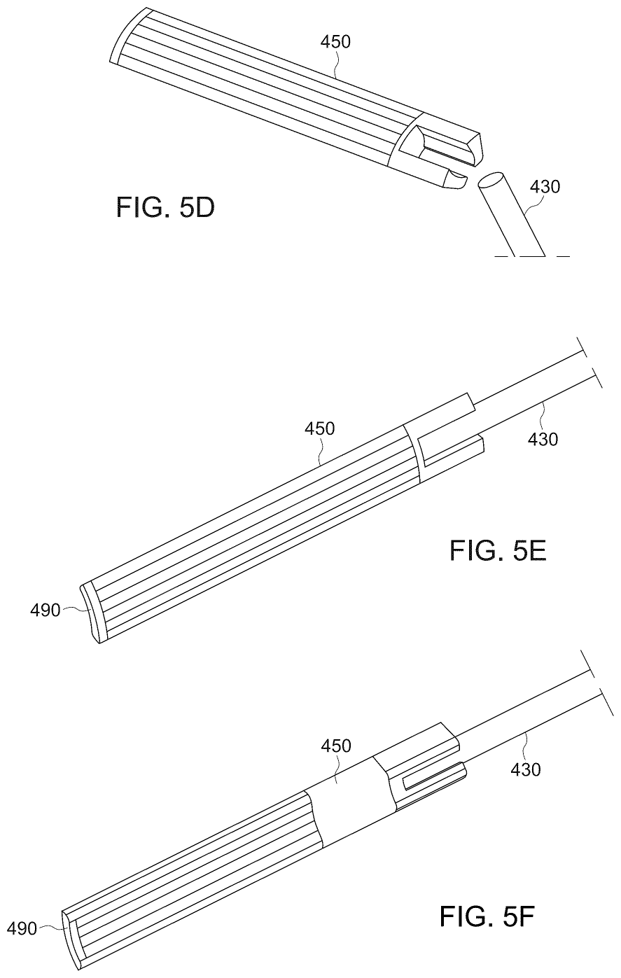

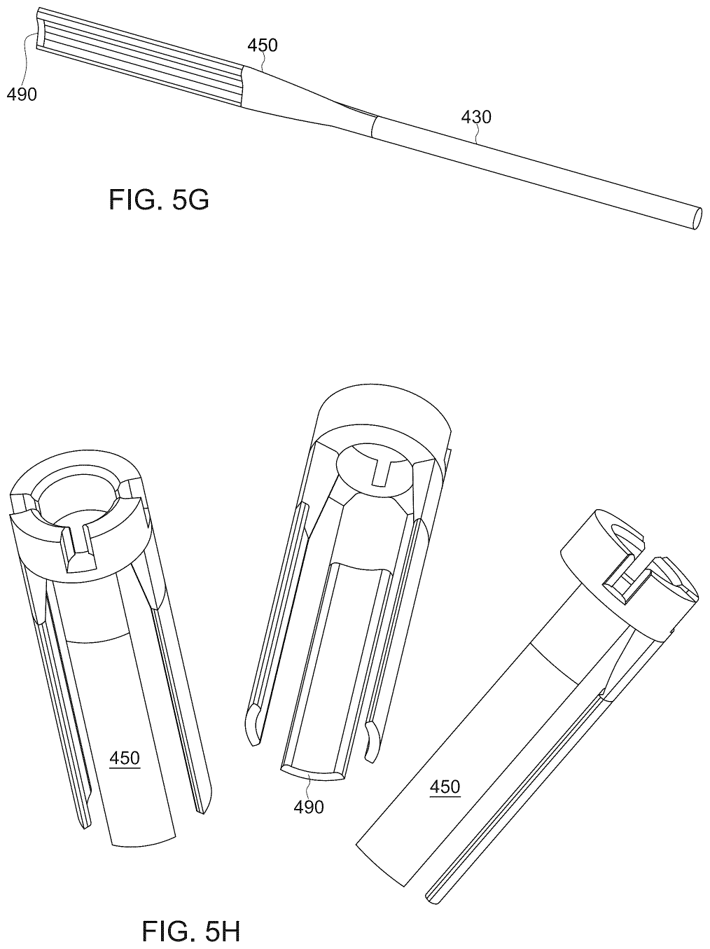

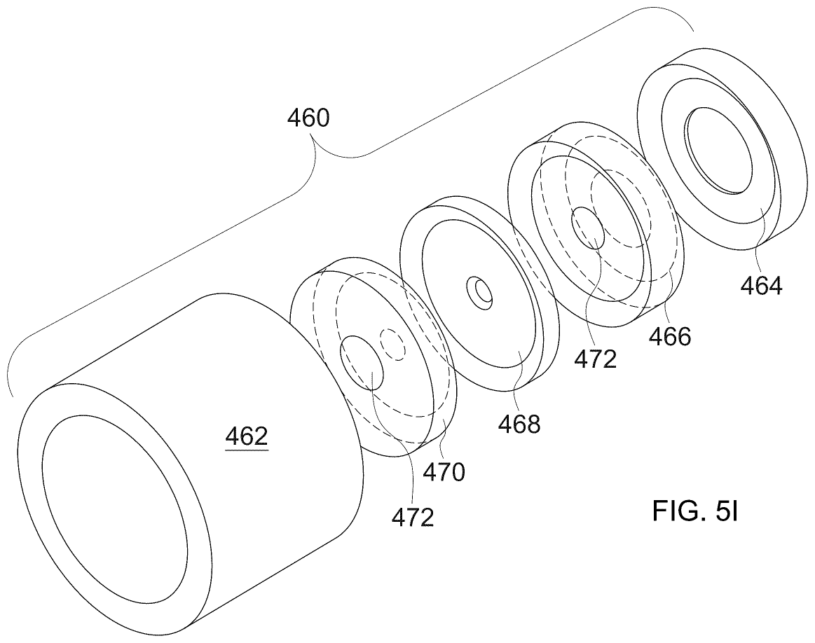

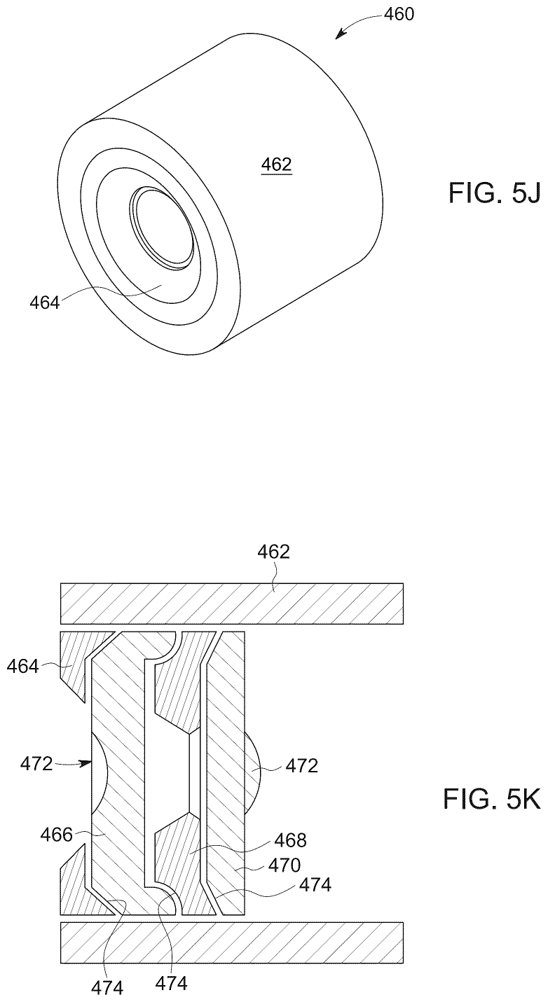

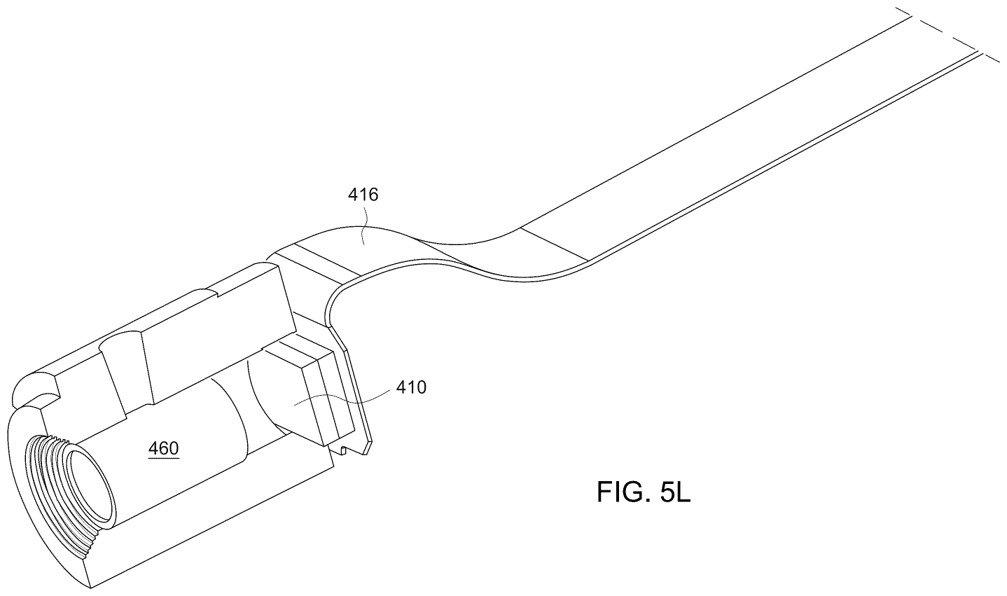

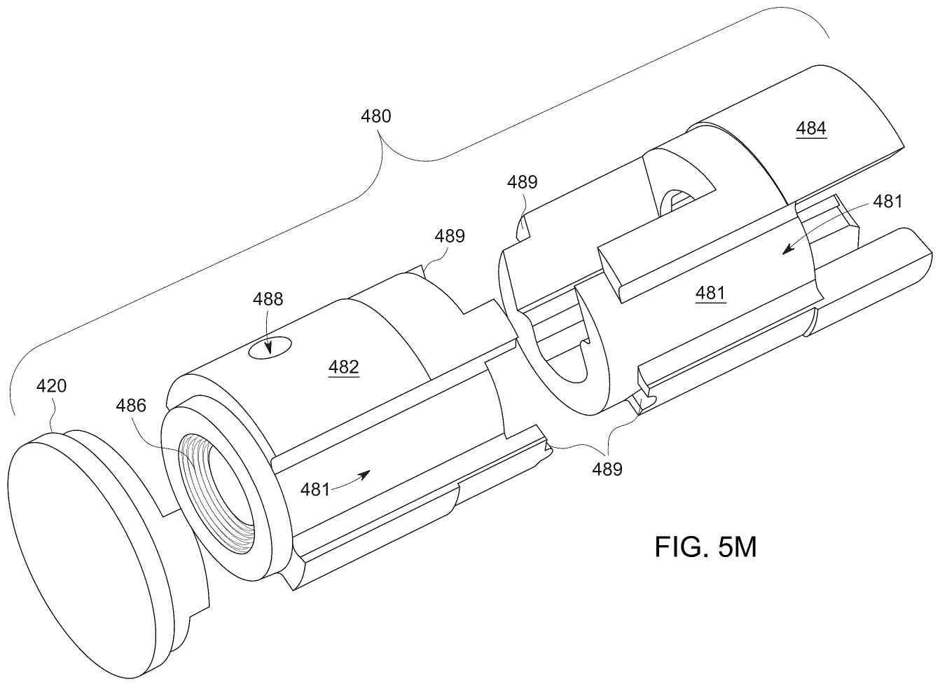

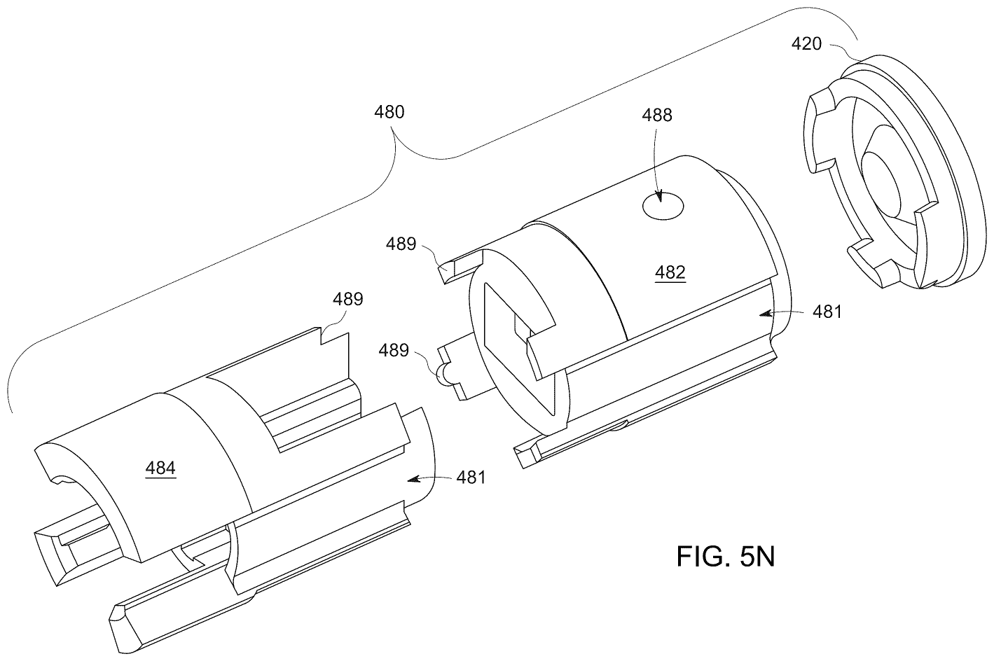

Referring to FIGS. 5A to 5H and 5P, light guides 450 may lie along the periphery of the tip components to conduct light from optical fibers 430 to the distal tip of the endoscope around the molded and image capture components of the tip. Light guides 450 may be made of clear optical plastic or glass. Outer diameter of shaft 110 may be designed to be small to reduce trauma and tissue injury, while camera 410 and other components can only be as small as permitted by technology. Thus, it may be desirable to reduce diameter of the light conveyance from the rear of the tip structure to the face of the tip, while maintaining a cross-sectional area sufficiently large to convey enough light. It may be desirable to arrange the illumination emission around a large fraction of the periphery of the scope tip to provide uniform lighting. Referring to FIGS. 5A, 5B, 5C, 5D, 5E, and 5F, three or four light guides 450 with an arcuate cross section may be arranged around the circumference of the tip. Each may accept one or more light fibers 430 that conveyed light from LEDs in the handle or control box.

FIG. 5B shows light guide 450 in cross-section, showing the narrowing from a larger-diameter fiber 430 (perhaps 1 mm) at the point of connection to light fiber 430 down to a thickness of about ½ mm or less to squeeze the light flow between the chassis components 438, 480 in the center, and outer metal shaft 110. The angle of narrowing section 452 may be less than the internal reflection angle of the material of light guide 450. In narrowing area 452, the surfaces may be highly polished, to effect high internal reflection. The axis of fiber 430 may enter at the center of the thickness of light guide 450. Under this arrangement, the cross section of 1 mm diameter fiber 430 and the cross-section of light guide 450 may be about equal, with narrowing region 452 designed to capture all of the light from fiber 430 and direct it into light guide 450, through a channel narrowed to about 0.5 mm between camera chassis 438, 480 and the wall of the shaft 110.

Referring to FIG. 5C, the surface of light guide 450 may be concave fluted or scalloped 454. The entry 456 into the fluted region at the proximal end of the flutes 454 may be tapered at an angle less than the angle of total internal reflection of the material Fluting 454 may provide two advantages.

First, fluting 454 may encourage the light to organize to flow down light guide 450 as the light emerges from narrowing region 452 of light guide 450. A light conduit in the region to conduct light past the constriction of the camera at the tip of the endoscope may balance two somewhat-contradictory conditions: it is desirable that the maximum light flow through light guide 450, and simultaneously, illumination light should be dispersed across the entire field of view of camera 410. Dispersion of fibers alone are limited by the numerical aperture (the angle of dispersion or collection at the two ends of the fiber), which is the angle of internal reflection, which in turn is governed by the difference in index of refraction between the core layer of the fiber, the cladding layer, and the ambient material around the fiber (typically air). Dispersion greater than the numerical aperture of the fiber may allow illumination light to reduce darkness at the edges of the field-of-view of the lens (roughly 70°) of camera 410. Fluting/scalloping 454 may create an appropriate level of dispersion within light guide 450, so that when the light emerges from light guide 450, the dispersion at the tip of the scope may nearly match the field-of-view of lens 460.

Second, organizing light guide 450 so that contact between the inner surface of shaft 110 and the outer surface of the plastic light guide, and between the inner surface of light guide 450 and outer surface of chassis 438, 480 occur on line contacts, which may reduce light leakage. The wall of light fiber 430 has two internal reflectance interfaces, one between the core and cladding layers of fiber 430, and one between fiber 430 and the external air. The shape of light guide 450 may be designed to increase air surround, and to reduce contact with epoxy or other materials, in order to reduce light leakage.

Light guides 450 may permit easier and higher-throughput direction of illumination light in a 30°, 45°, 60°, or 700 offset scope, which may reduce the brightness, power consumption, and heating at the illumination LEDs.

Referring to FIGS. 5D, 5E, and 5F, light guide 450 may be formed to accept fiber 430. A coupling may be arranged to capture and transfer as much light as possible from fiber 430 to light guide 450, and may be arranged to structurally retain fiber 430 to ease assembly.

Referring to FIG. 5G, light guide 450 may be formed by deforming fiber 430 itself. This may effect a higher-throughput coupling from fiber 430 to light guide 450, and greater angle of view and uniformity of the light coming out of the fiber. than butt-coupling of FIGS. 5D, 5E, and 5F. The light fiber may be deformed by gentle heating, for example, in steam, and then applying pressure. The pressure may be applied in a highly polished mold, to provide a highly smooth surface. A shaped fiber end may provide greater tolerance to small misalignments during manufacture, compared to the high precision placement required for butt-coupling.

Referring to FIG. 5H, three light guides 450 may be molded together as a single part. This may ease assembly in two respects. First, instead of assembling three small parts, this technique may permit an assembly process to manage one larger part. Second, the three prongs of light guide 450 may hold the entire assembly of front chassis 482, rear chassis 484, lens and filter assembly 460, image sensor 410, and flex board 416 (FIGS. 5M, 5N, and 5O) together into a unit for further assembly.

Referring to FIGS. 5I, 5J, and 5K, lens assembly 460 may be formed in a tube 462 that encloses an end cap 464, a first lens 466, a spacer/iris 468, and a second lens 470, and a filter. Circular parts 464, 466, 468, 470 may be about 1 mm or 1.2 mm in diameter. To ease reliable assembly, shapes 474 embody poka-yoke principles so that they will only stack one way. For example, the conic angle, straight-vs-curvature, etc. 474 may vary at the different joints, so that the parts cannot be assembled in incorrect orders. For the two lens parts 466, 470, the lens itself is only the center circle portion 472 (which appears similar to an eye cornea in FIG. 5I). The optical lens is shown in FIG. 5K as the depression 472 in the front lens and raised bubble 472 in the rear lens. Center spacer 468 may have a precise lateral depth to ensure correct spacing between the two lenses 466, 470, and a relatively small center aperture to block excess light. Outer cap 464 may function as a positioning spacer, as a flange to capture the other parts, and/or to block excess light. Excess light to be blocked may be light leaking from light guides 450, or may be light reflected indirectly from within the surgical cavity but outside the image area. Excess light may be blocked so that it that does not degrade image quality.

Referring to FIG. 5L, image sensor 410 may be mounted on flex circuit board 416. Referring to FIGS. 5M, 5N, a tip may be formed using front chassis 482 and rear chassis 484, which in turn, hold camera 410 and a cover window in place. Front and rear chassis parts 482, 484 may be molded as single parts of opaque plastic or may be made of machined aluminum. The sides of the front and rear chassis 482, 484 may have channels 481 that hold light guides 450 to the periphery of the chassis 480. The front and rear chassis 482, 484 may have features 489 at the joints that mate in only one way (for example a round protrusion on front chassis 482 that mates with a round recess in the rear chassis, and squared-off mating parts to ensure angular reproducibility). Front chassis 482 may have a stepped cone aperture 486 to reduce stray light interference reaching the camera. Rear chassis 484 may have an opening so that it does not contact light guide 450 in the narrowing region, because the internal reflection angle of the fiber optic components is higher when backed against air than when backed against plastic. Lens assembly (460 from FIGS. 5J, 5K, 5M, and 5N) may be mounted in front chassis 482. Then rear chassis 484 may be slid over the length of circuit board 416 so that circuit board 416 extends through the center hole of rear chassis part 484. Then the image sensor 410/circuit board 416 may be mounted to front chassis 482. Then rear chassis 484 may be mated to front chassis 482, which holds lens assembly 460, camera 410, and board 416 in place relative to the two chassis parts 482, 484. This approach may reduce bending of board 416, which may reduce risk of straining the flex board 416 and its electronics, but still builds some slack and strain relief into the assembly.

The lens assembly may include an IR cut filter to remove unwanted IR from entering the image sensor.

Alternatively, the lens and filter elements may be adhered directly to the image sensor. The spacing between the image sensor and the lens and filter elements may be controlled via glass beads of a diameter matching the desired spacing.

Chassis 480, 482, 484 may in turn mount a clear window. Window 420 may be molded and glued in place, or may be overmolded last as the molding step that holds the other components together. Light may be communicated from light fibers to the face of the scope via light guides 450.

At this point, placement of lens 460 may be calibrated. In some cases, lens tube 462 may be made of ferromagnetic or paramagnetic material, so that magnets may be used to move lens assembly 460 within the front chassis 482 to focus the lens on image sensor 410 to improve focus, focal range, and field of view. In other cases, components of the mounting brace/chassis 412, 414, 438 may be threaded to assist in a focus adjustment. As shown in FIGS. 5M and 5N, a drop of adhesive may be applied through a microneedle through weep-hole 488 to secure the lens assembly in place. This also seals weep hole 488 against fluid intrusion. Then light guides 450 of FIGS. 5A to 5H may be added in channels 481 at the sides of chassis 480, and the window 420 may be added on the front of chassis 480. The assembly is shown in FIG. 5P.

Referring to FIG. 5O, the window part may have a step to engage with the edge of shaft tube 110. The step labyrinth may provide sealing against intrusion by fluids and pressurized atmosphere that is typical to surgical insufflation. In some case, the window may be secured and sealed with adhesive.

The front and rear chassis 480, 482, 484 then hold lens and filter assembly 460, camera/image sensor 410, and flex board 416 and hold them in proper spatial relation within shaft 110. This reduces part count. Chassis 480 may hold all of the components together in an assembly that can be mounted in shaft 110 in a single operation, which may ease manufacturability. The parts 474, 489, may use poka-yoke design techniques, so that the configuration of the parts allows assembly only one way, and calls attention to errors before they propagate.

### V.D. Diffusion Terminal Surface

In some cases, the distal surface 490 of fibers 430 or light guide 450 may be roughened or coated with a diffusive coating, analogous to the coating used to coat the inside of soft white light bulbs. By diffusing the light at emission end 490 of fiber 430 or light guide 450, the dispersion angle may be increased, which increases the cone of illumination and width of field, and may reduce undesirable shadows and other artifacts. In some cases, dispersion may be accomplished by a holographic diffuser in fiber(s) 430 or light guide(s) 450. In other cases, a diffuser may be imposed by a random process such as sandblasting, molding against a sandblasted surface, or by some similar random process. In other cases, one or more texture patterns may be photo-etched in the steel of the mold for the tip of the a fiber(s) or light guide(s) 450. One example texture may be a series of micro-domes, small circular features each having a lens profile designed to diffuse light. The microdomes may be randomly placed and of random size to avoid collimation or diffraction in specific directions, which could result in cold spots. In some cases, distal surface 490 may be roughened by a rough grinding process, analogous to the early stages of grinding a lens. Opal glass may be embedded in distal end 490 of light guide 450. The distal end 490 may be textured with other diffusion patterns such as circles, lines, or hexagons.

### VI. Antifouling and Antifogging

### VI.A. Heating

Lenses may also be fogged by condensation of water vapor from the body cavity that is being operated on. Endoscope tip 400 may be the coolest point in the bodily cavity because it is nonliving tissue with no metabolism, and because operating rooms are typically kept quite cool and the stainless steel insertion shaft conducts that cold from the ambient room air to the tip. In some cases, the tip of the endoscope may be heated. In some cases, illumination LED 418 may provide slight heating, which may reduce condensation and fogging on the tip. The heating need only be by a few degrees, enough to ensure that the endoscope is not the coolest point in the cavity. In some cases, apertures for insufflation fluid (saline, carbon dioxide, or other, as the case may be) may be oriented to direct the fluid flow over the window surface to provide additional cleaning.

### VI.B. For Endoscope's Delivery Packaging, Vial of Fluid to Protective a Coating on the Endoscope's Lens/Window

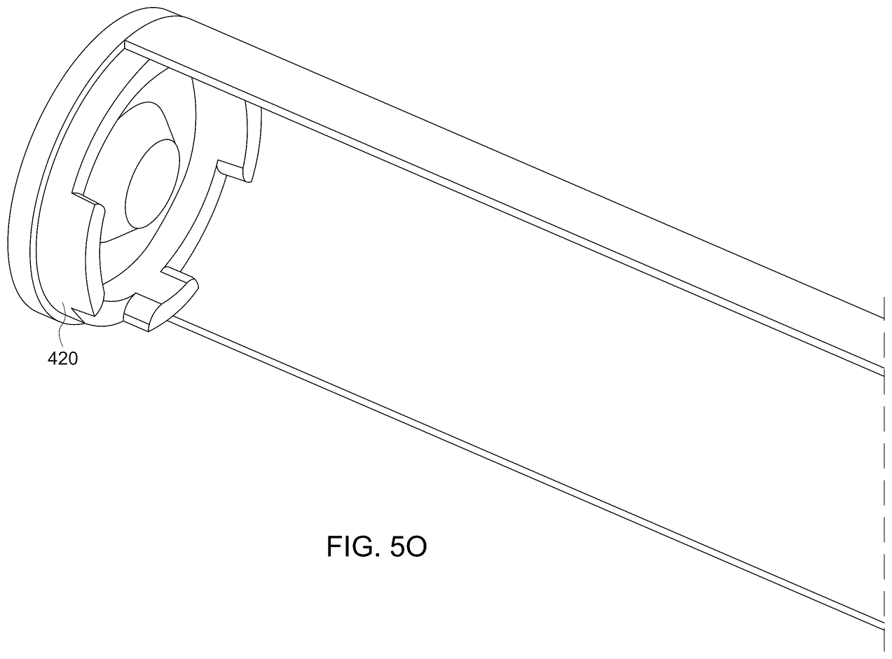

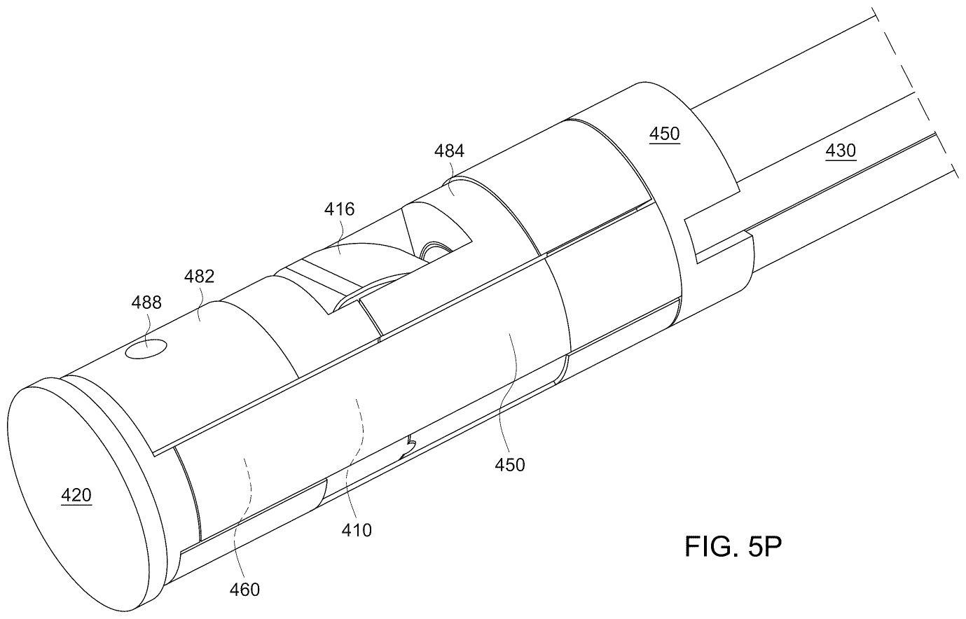

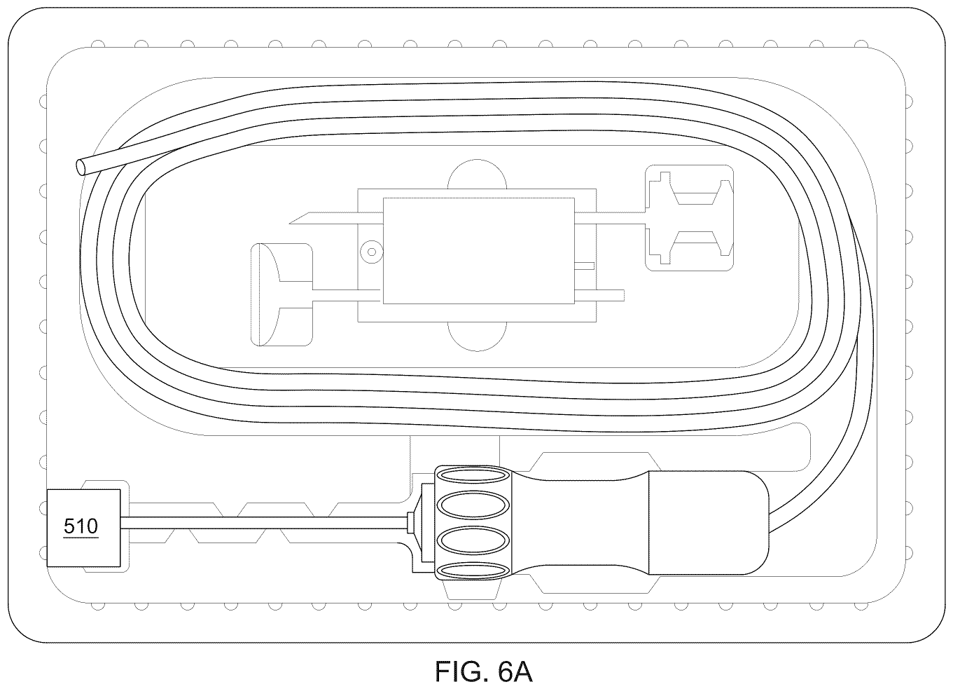

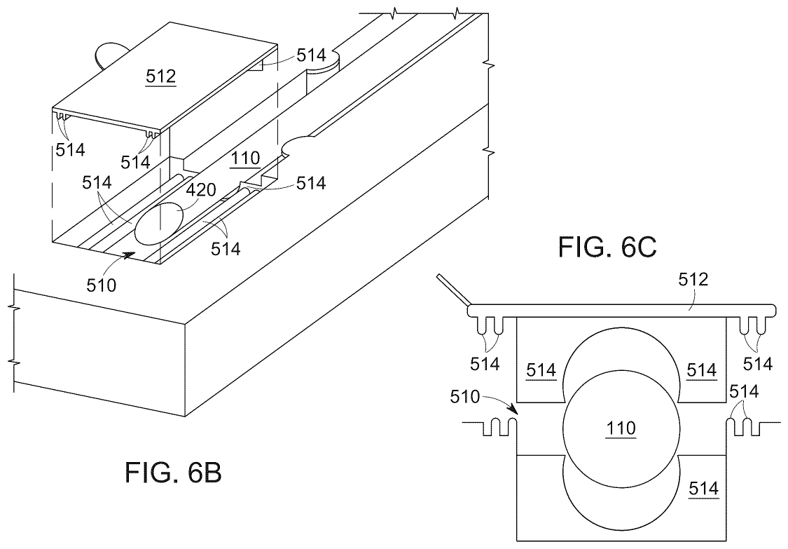

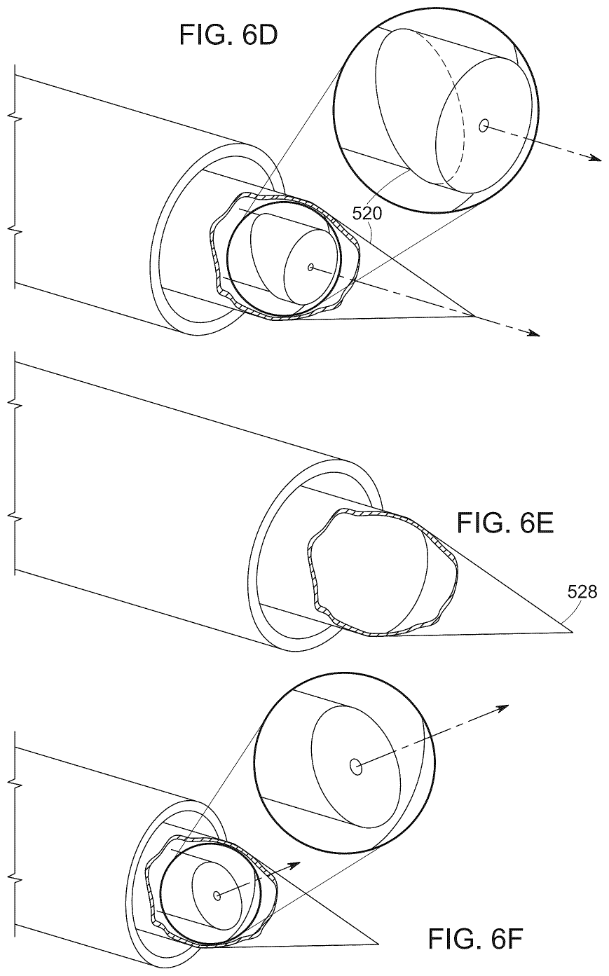

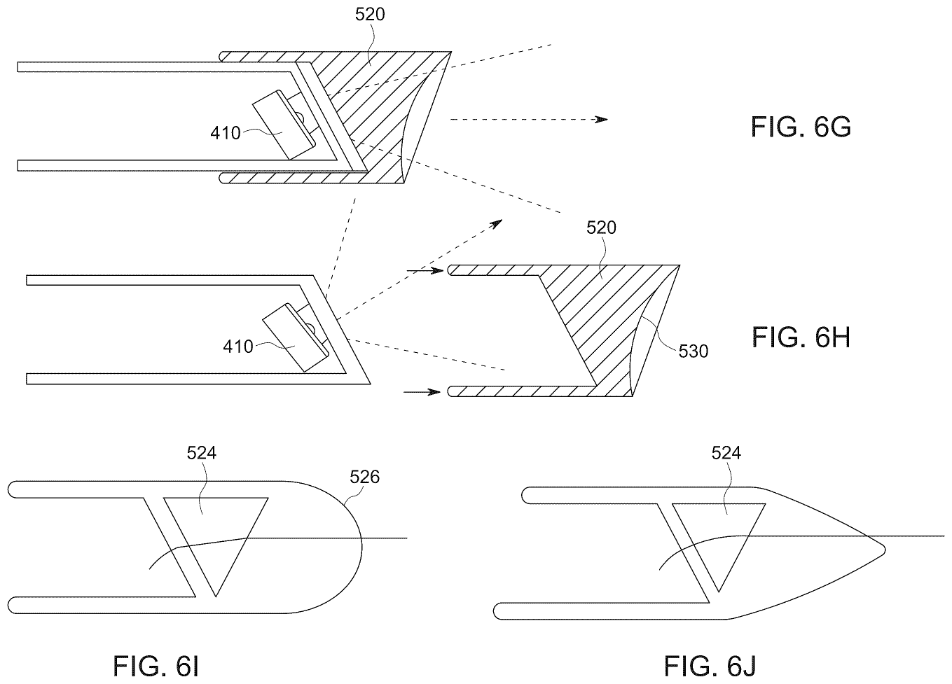

Referring to FIGS. 6A to 6C, optical lenses/windows of obturator 104 and/or endoscope may be coated with an anti-adhesive coating. Shipping or delivery packaging for endoscope 100 and/or obturator 104 may incorporate a well, vial, or other containment structure 510 to hold an anti-adhesive lubricant in contact with the coating on the objective lens or window 420 of the endoscope between the time of manufacture or refurbishing up to the moment of use, to improve the coating. Well/vial 510 may have a cap that retains the lubricant in contact with the lens/window matrix surface, to preserve the coating. The cap may have a seal against the outside environment, and a seal that seals around a shaft of the endoscope. The lubricant may be held in place over the endoscope lens/window by a rigid cap, or a flexible condom.

A window 430 of endoscope 100 may have a coating to enhance optical or mechanical properties of the endoscope, and packaging for the endoscope may incorporate a vial or well or cap 510 of lubricant to maintain infusion into the retention matrix. The coating may be an anti-adhesive coating to reduce accumulation of contaminants on the surface of the lens/window, so that forward view of the endoscope remains clear. The anti-adhesive coating may be applied in two steps, first to build a porous matrix or network for retention of a lubricant on the surface of the lens/window, and then the lubricant. The lubricant may be an oil, or other liquid or gel, so that the lubricant acts as a liquid-on-liquid surface. The infused liquid may be a silicone oil (Momentive or Gelest polydimethylsiloxanes, such as 10 cSt, 350 cSt, 500 cSt), perfluorinated fluid (perfluoroperhydrophenanthrene, or Vitreon, and perfluoropolyethers or (PFPEs) of 80 cSt to 550 cSt: DuPont Krytox series), or other liquid or gel with appropriate combinations of high transparency, low surface energy, appropriate viscosity and volatility so it will be retained on the surface, chemical inertness, and prior US Food and Drug Administration (FDA)-approval. Suitable anti-adhesive coatings are described in Thin Layer Perfluorocarbon (TLP) coating developed by the Hansjörg Wyss Institute for Biologically Inspired Engineering at Harvard University, described at https://wyss.harvard.edu/technology/tlp-a-non-stick-coating-for-medical-devices (incorporated by reference), and in U.S. patent application Ser. No. 16/069,220, Anti-Fouling Endoscopes and Uses Thereof, filed Oct. 24, 2018 (incorporated by reference), and commercially developed as Slippery Liquid-Infused Porous Surfaces (SLIPS) coatings and other repellent coatings and additives by Adaptive Surface Technologies, Inc. of Cambridge, MA, described at https://adaptivesurface.tech and its subsidiary pages (incorporated by reference), and as described in Steffi Sunny, et al., Transparent antifouling material for improved operative field visibility in endoscopy, Proceedings of the National Academy of Sciences U.S.A., 2016 Oct. 18; 113(42):11676-11681. doi: 10.1073/pnas.1605272113 (Sep. 29, 2016) (incorporated by reference).

The well or vial may be formed so that the cover includes appropriate seals 512 to retain the protective oil or gel. For example, edges around a cover may have a labyrinth seal. Two components of an end wall may each embrace slightly more than 180 degrees of the endoscope shaft so as to form a seal. Two components of an end way may have labyrinth seals against each other. The nature of the seal 512 may vary depending on the viscosity of the lubricant, and the surface energy of the lubricant vis-à-vis the material of the cap.

When endoscope 100 is placed in use, any excess of the lubricant may be wiped off. If the lubricant is bio-inert and nontoxic, it may be left in place to protect the lens during some phase of penetration.

Referring again to FIGS. 1J and 2E, the shipping, delivery, or storage case for the endoscope may have compartments for one or more scope insertion shafts, for a handle, for various hoses and cords, obturator 104, in addition to the well or vial or other retention for anti-adhesive lubricant.

### VII. Lens Cap with Optical Correction for Use During Insertion of Offset-View Endoscope

Referring to FIGS. 6D to 6J, end cap 520 may be affixed to the end of the endoscope to alter a field-of-view angle so that a single endoscope may be used at two different phases of the surgical procedure. During penetration using an endoscope with a 30° offset of view angle, the endoscope may be fitted with a refractive cap or a prism, that bends light to reduce the offset angle. Refraction by refractive cap 520 may yield a 0° on-axis view, or a near-zero angle such as 3°, 5°, or 10°. A scope with refractive cap 520 may be used during initial penetration by trocar 102 and obturator 104, to give a straight-ahead view, or a near-straight-ahead view to within 3°, 5°, or 10°. Referring to FIGS. 6E and 6H, once trocar 102 and obturator 104 have reached the site of the surgery, refractive cap 520 may be removed from endoscope 100 to yield a 30° offset scope. Removal may require withdrawal of endoscope 100, and then reinsertion. Then, referring to FIG. 6F, endoscope 100 may be returned down trocar 102 for use in a surgical procedure with a 30° offset field of view. Typically, obturator 104 is discarded once the penetration pierce is completed.

Refractive cap 520 may be molded of clear plastic, such as polycarbonate, acrylic, styrene, polyolefin, silicone, or inorganic transparent materials such as silica (silicon dioxide). In some cases, the refractive cap may be formed of multiple materials, such as glass and plastic, or two different plastic resins, to marry light refraction and various mechanical functions, such as to provide a sharp piercing point for cases where the endoscope is used without an obturator. Referring to FIGS. 61 and 6J, refractive cap 520 may be formed of different materials that provide different properties. For example, a high-index-of-refraction prism 524 may be embedded in a plastic of lower index of refraction, so that the plastic may be shaped in a desired manner. Refractive cap 520 may be shaped to provide an atraumatic rounded nose 526, to avoid tissue injury. In other cases, FIGS. 6D-6F, 6J, the low-refraction plastic may be shaped with a piercing point 528. The point may be reinforced by a steel piercing tip.

One surface or the other may be convex or concave 530 to widen or narrow the field of view, or to magnify or reduce the forward-looking view. Refractive cap 520 may permit a single endoscope to be used in two different phases of the surgery, where two different views are desired. The degree of refraction may be enough to reduce the offset angle by 5°, 10°, 15° 20°, 25°, or 30°. In some cases, obtaining partial correction less than 0° on-axis may be sufficient to improve the view during piercing. In some cases, the apex point of obturator 104 may create optical distortion, so it may be desirable to maintain some optical offset to keep that distortion away from the center of view.

Refractive cap 520 may be attached to the tip of endoscope 100 by friction fit or interference fit (the inner diameter of refractive cap 520 equal to or slightly smaller than the outer diameter of endoscope tip 400), by means of a weak or snap-frangible adhesive, via a small bump on the inner diameter of refractive cap 520 that engages with a depression or dimple in the tip of endoscope 100, a threading or channel on the inner diameter of refractive cap 520 to engage with a small raised stud on endoscope 100, or by other connector. The sleeve portion of refractive cap 520 may be formed of a heat-shrink plastic or other material that can be shrunk to secure the connection. Refractive cap 520 may be held in place by a sleeve of elastic plastic, like a condom. While it is not desirable that refractive cap 520 fall off during use, that is a low severity event, because the endoscope with refractive cap 520 is inside obturator 104, and the refractive cap will be captured and removed when obturator 104 is withdrawn.

Refractive cap 520 may have holes through the attachment sleeve to permit fluid flow and/or suction to flow through from passages in the endoscope shaft.

A surface of refractive cap 520 may be etched with a reticule or measuring scale. The reticule may be marked with a scale with which the surgeon can measure the size of objects seen through the endoscope. The surgeon may also use the reticule to align the endoscope within the surgical field.

Refractive cap 520 may have optical filters, for example, to reduce light reflected into the endoscope, to block certain wavelengths of light. The filter may include a polarizing filter, a bandpass filter, a color filter, or an interference filter. These filters can be used in conjunction with specialized light sources (e.g., ultraviolet, infrared, or polarized) and video processing for therapeutic and diagnostic purposes. Thus, refractive cap 520 may be part of a complete system to diagnose pathology using different wavelengths of light and/or colors of light and filtering the light. Further, refractive cap 520 may also be provided as part of system that delivers photonic energy to a surgical site to control and visualize photodynamic therapy.

Referring again to FIG. 6G, in some cases, objective lens/window of the endoscope may be treated with a coating, for example an anti-adhesive coating, and the endoscope may be delivered with anti-adhesive lubricant in a space between the lens/window and the proximal surface of the refractive cap's prism. In such cases, the endoscope may be used as shown in FIGS. 6D, 6E, and 6F, first with refractive cap 520 attached to provide a close-to −0° view offset. Then refractive cap 520 may be removed, leaving endoscope with its 300 offset, with the anti-adhesive-coated lens/window exposed into the surgical cavity.

### VIII. Avoiding Fasteners, Springs, and Other Small Components

Moving parts and structural components of endoscope 100 may be joined and affixed in place using techniques that avoid small components such as fasteners and springs. These manufacturing techniques may reduce costs of molding, reduce costs of assembly, reduce manufacturing steps, and reduce the likelihood that a separate component, for example, a steel spring or screw, may come loose and endanger patient safety. Likewise, assembly may be accomplished without adhesives or solvents that may be toxic.

VIII.A. A Button with Embedded Springs

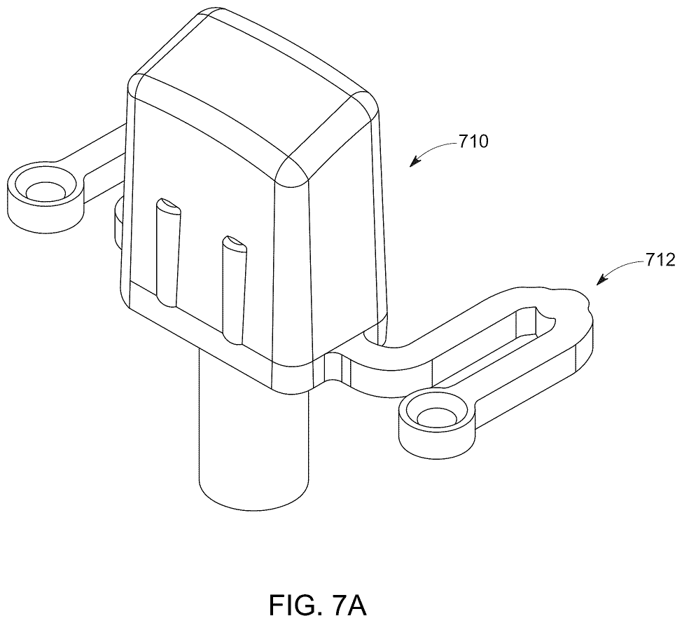

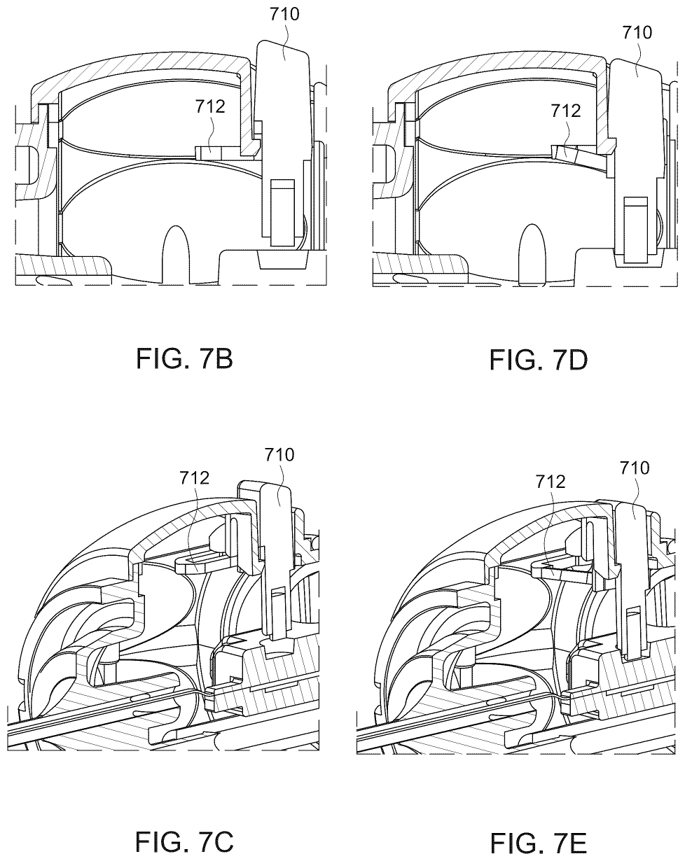

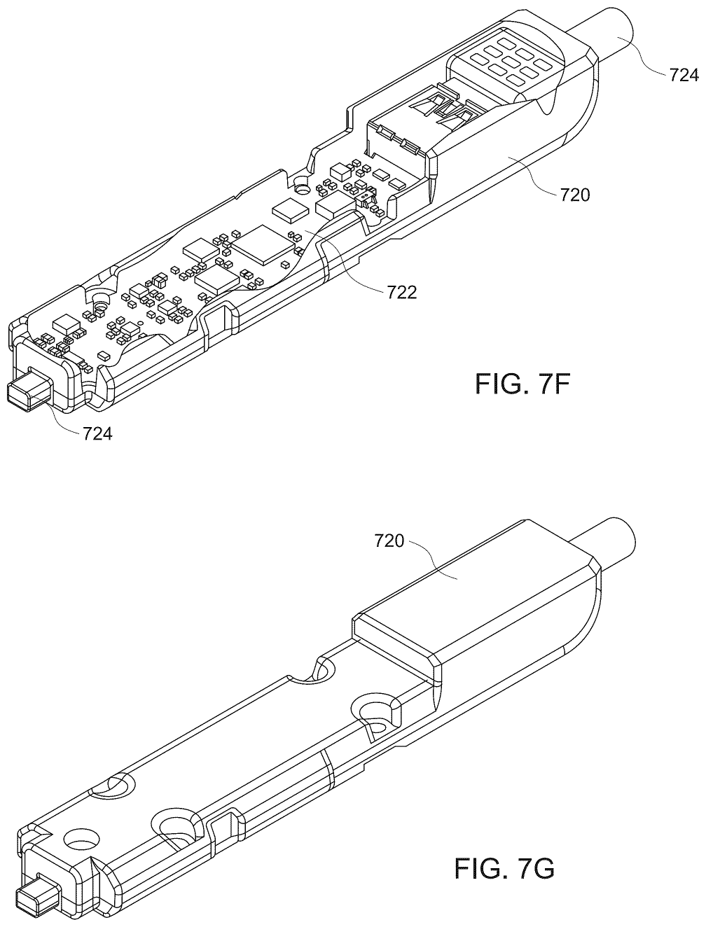

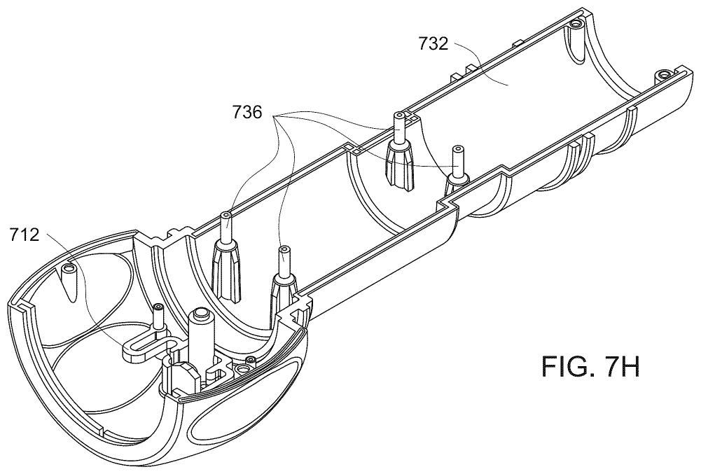

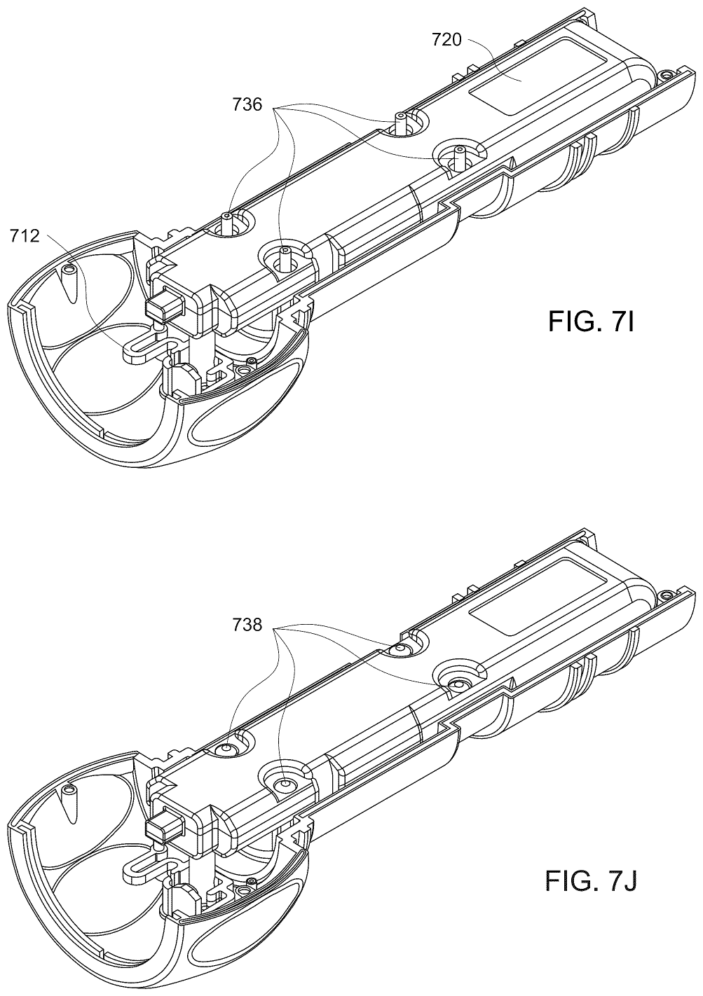

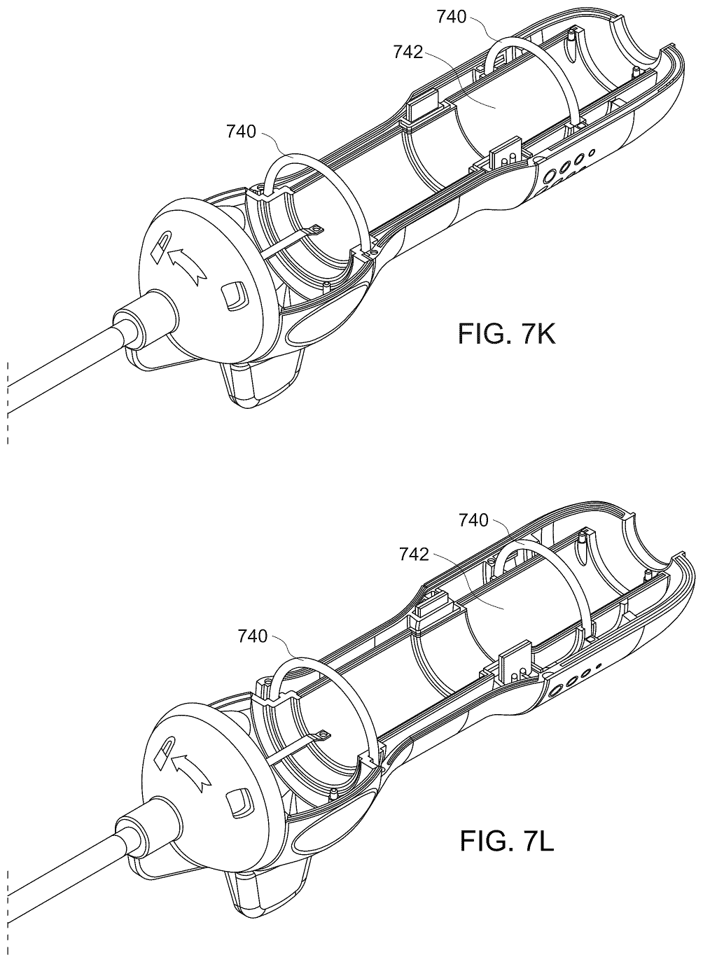

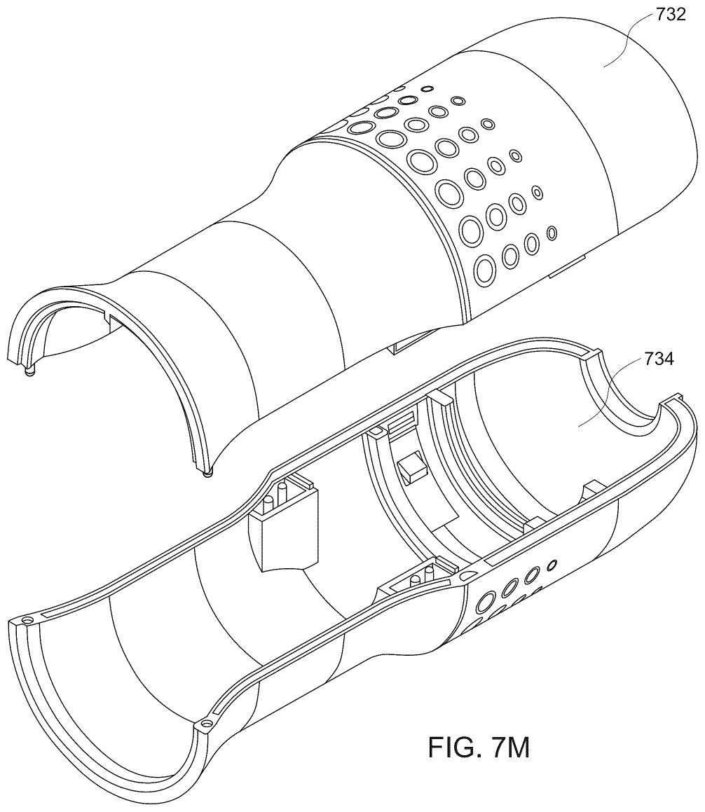

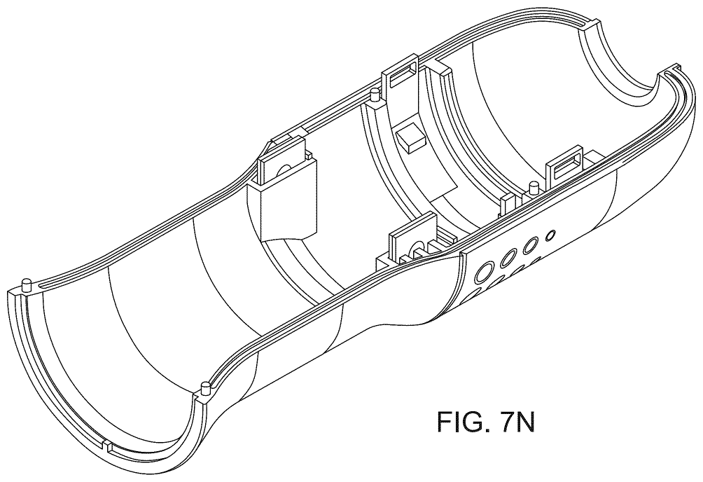

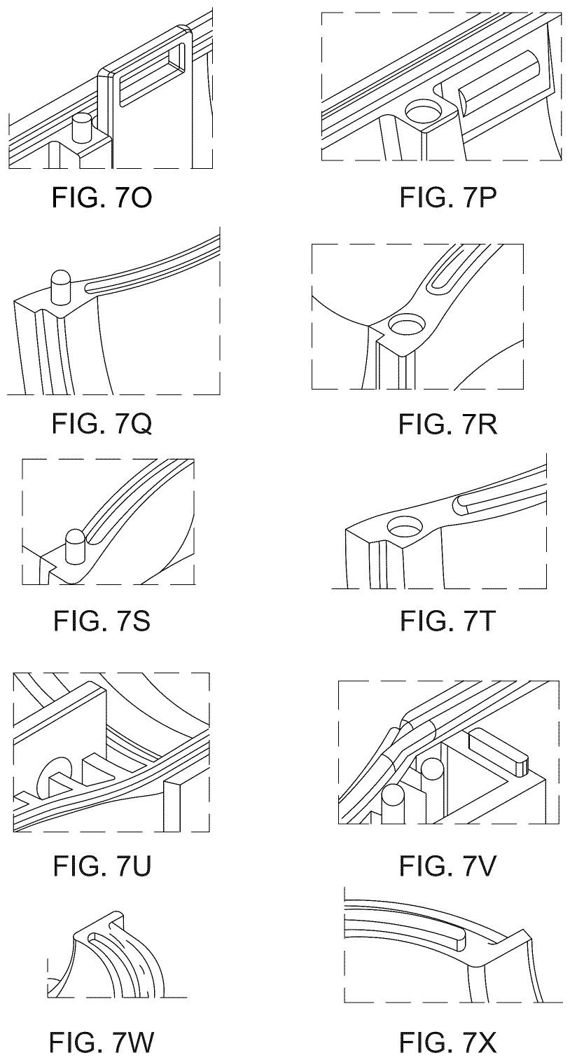

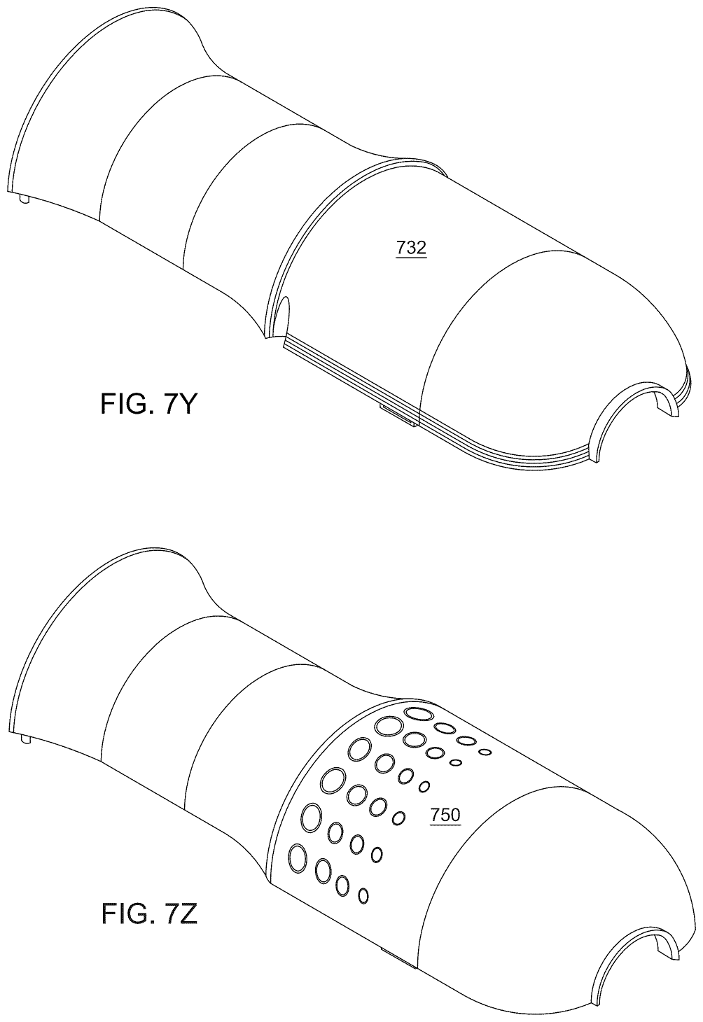

Referring to FIG. 7A, a control button 710 for the endoscope may be formed as a single component with the button, its guide track, and its springs 712 molded as a single component.

This single component may be manufactured to simultaneously provide adequate stiffness so that a button press is transmitted from the top of the button to the bottom, and adequate resilience in beam structures 712 to provide restoring force to return the button to its undepressed state. The loop beam structures 712 may be molded with flexible projections extending out from a united molded component. These extended flexible projections 712 may act in a combination of bending and torsion so that when button 710 is released, elastic memory of the materials will spring the button back into its initial position. FIGS. 7B and 7C show the button in its normally-up position, with the spring loops 712 in their originally-molded planar configuration. FIGS. 7D and 7E show the button depressed, and the spring loop 712 deformed in torsion.

Overmolding (see § VIII.B) of the handle's circuit board may be used to mechanically position and secure the button's positions as a guide, and ensure that a magnet at the bottom of the button shaft correlates to the area of the senor located on the handle board.

The button with its springs may be molded of ABS plastic.

In some cases, the handle may have metal fasteners and other small metal parts, or assembly may use heat staking that may be melted to retain the springs of the button, chemical adhesives, or the like, but they may be encapsulated fully or partially within overmolding sufficiently to ensure no loosening, escape, or contact with fluids that flow into a patient.

### VIII.B. Overmolding of Case Over Circuit Board

Referring to FIGS. 7F and 7G, a circuit board 722 to be mounted within the handle, with its connecting cables that will feed forward into the insertion shaft, and its connecting cables that will feed backwards to the supporting power supply and computer driver, may be overmolded 720 using low pressure overmolding. Overmolding 720 may include through-holes that provide secure mechanical mounting points for mounting within the handle. Overmolding 720 may provide a covering that protects all sides and is resilient to protect circuit board 722 from mechanical insult during manufacturing and use. Overmolded case 720 may provide strain relief for the cables 724 at the two ends of circuit board 722, to reduce damage that might be caused by pulling on the cables 724. The overmolding 720 may provide a unified, no-seams covering to protect he circuit board 722 from water intrusion (saline is very conductive, so reliable electrical insulation should be ensured). Overmolding 720 may protect against fluid ingress to a level of IP65 under international standard EN 60529. Liquid-tight protection of overmolded case 720 over circuit board 722 (and any other liquid sensitive components within the handle) may allow the sterilization of the interior of the handle via flowing a sterilizing liquid or gas through the interior of the handle, and may provide dielectric protection to prevent electrical shock to the patient. The overmold may use a biocompatible low-pressure injection mold compound with a high dielectric constant, about 5 mm thickness. In some cases, the overmold may have good thermal conductivity to disperse heat from high power consumption electronic parts. The dielectric of the overmold may be chosen to help protection against electrostatic discharge. These advantages may be achieved by reducing manufacturing to a single manufacturing step.

### VIII.C. Avoiding Internal Fasteners

Referring to FIG. 7H, the overmolded circuit board structure may be mounted within the handle via molded clips, heat staking, and similar techniques that result in a unitary structure, without separate small-part fasteners. In FIG. 7H, the inner shell of the handle may be molded in two parts 732, 734. Top half 732, shown turned upside-down in FIG. 7I, may be molded with several molded bosses 736. In FIG. 7H, the button has been turned upside-down and set in place; the looping resilient projections are visible. In FIG. 7I, overmolded PC board 720 may be placed over bosses 736, so that bosses 736 project through the through-holes in the board casing. In FIG. 7J, heat and/or ultrasonic vibration may be used to melt the tips of bosses 736 into domes or heads 738 that hold the button and PC board in place.

By these techniques, endoscope 100 may be assembled into a structure that is strong, without small parts that require separate assembly steps or that can become dislodged, and without toxic adhesives or solvents.

### VIII.D. Rotational Resistance Via O-Rings

Referring to FIGS. 7K and 7L, inner shell 742 and outer handle shells 732, 734 rotate relative to each other, so that the surgeon may adjust field of view, using the magnetic Hall effect sensors 324 discussed above. The inner handle 742 rotates in unison with rotation collar 112. Inner handle 742 and rotation collar rotate relative to outer handle 114. The inner handle 742 and outer handle shells 732, 734 may be coupled via two silicone O-rings 740 that provide some friction to maintain their relative position, while allowing the surgeon to rotate the two handle components to adjust the arthroscope's field of view.

For assembly, the cylindrical inner handle may be fully assembled, and the O-rings 740 may be fitted over the end of inner handle 742 into their two retaining channels. Then the two halves of the outer handle shell 732, 734 may be brought together like buns over a hotdog, surrounding the inner shell and O-rings 740. Then the two halves of the outer shell 732, 734 may be ultrasonically welded to each other.

### VIII.E. Ultrasonic Welding of the Two Halves of the Outer Handle Shell

Referring to FIGS. 7M and 7N, the two halves 732, 734 of the outer shell may be molded of biocompatible plastic resin. All components that have direct contact with the patient as well as indirect contact may be molded of biocompatible material. The two halves may be ultrasonically welded, laser welded, fusion welded, or over molded without adhesives or solvents, which may reduce patient exposure to toxins which tend to be higher in volatile adhesives or solvents than they are in solid plastic. Referring to FIGS. 7O and 7P, the two outer shell halves may have locking clips or hooks that may snap together to hold them temporarily before ultrasonic welding begins.

Referring to FIGS. 7O, 7P, 7Q, 7R, 7S, 7T, 7U, 7V, 7W, and 7X, various energy directors and troughs may be designed to ensure high efficacy of ultrasonic welding. In each case, the height of the energy director is slightly higher than the depth of its mating trough, and the width of the energy director is slightly narrower than the trough. Ultrasonic vibration may cause the energy director to slightly melt and deform to fill the trough. The ultrasonically-welded seams may come very close to fully enclosing the cavity of the outer handle, to reduce the likelihood of any fluid intrusion from outside into the interior of the endoscope. Any fluid carries risk of pathogens. Sterilization of the interior of devices such as endoscopes is difficult, so exclusion of fluids is important for any components that may be reused. Adhesives may be used to ensure ingress protection, particularly at points of the endoscope that are not contacted by fluids that flow into the patient.

### VIII.F. Thermoplastic Elastomer Coating Handle

Referring to FIGS. 7Y and 7Z, a high friction surface 750 may be bonded to or overmolded onto the outer surface of the outer handle shell 732, 734, to provide a high-friction contact between a surgeon's gloved hand and the handle. During surgery, the surgeon's glove and/or the handle may become covered with bodily fluids and other liquids that may reduce friction, so that the surgeon could have trouble maintaining control of a solid endoscope. By covering the endoscope handle with a high-friction compound 750, such as thermoplastic elastomer (TPE), the handle becomes higher friction, which may improve the surgeon's control. If the cover is overmolded as a single unitary layer, it may provide additional sealing against fluid intrusion. One medical grade, biocompatible TPE is Medalist MD-34940 or MD-34950 from Teknor Apex of Pawtucket, RI.

### IX. Liquid Flow

### IX.A. Liquid-Tight Seal

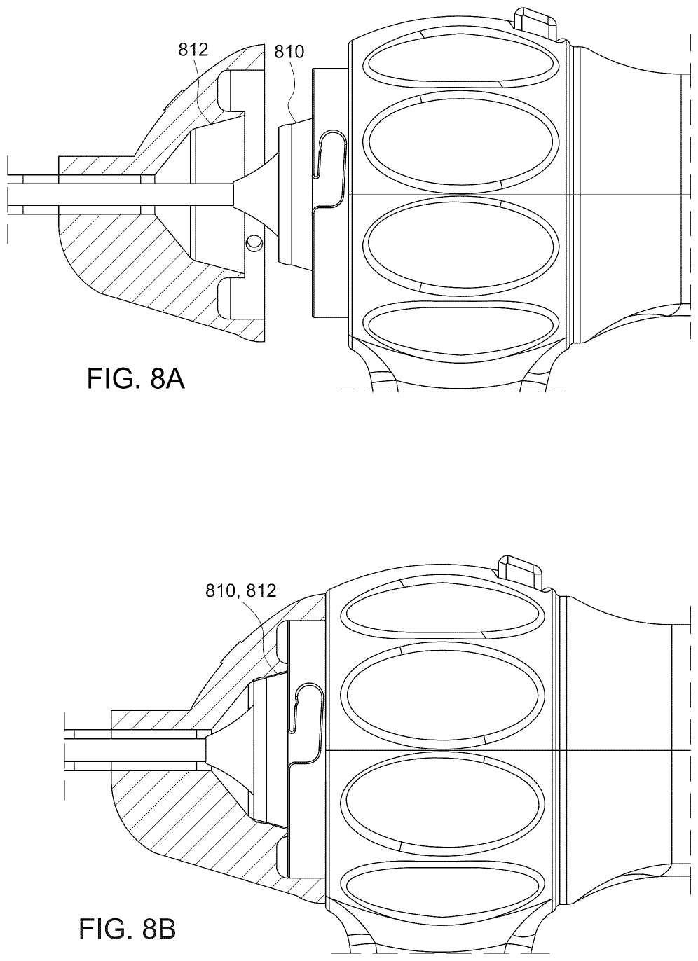

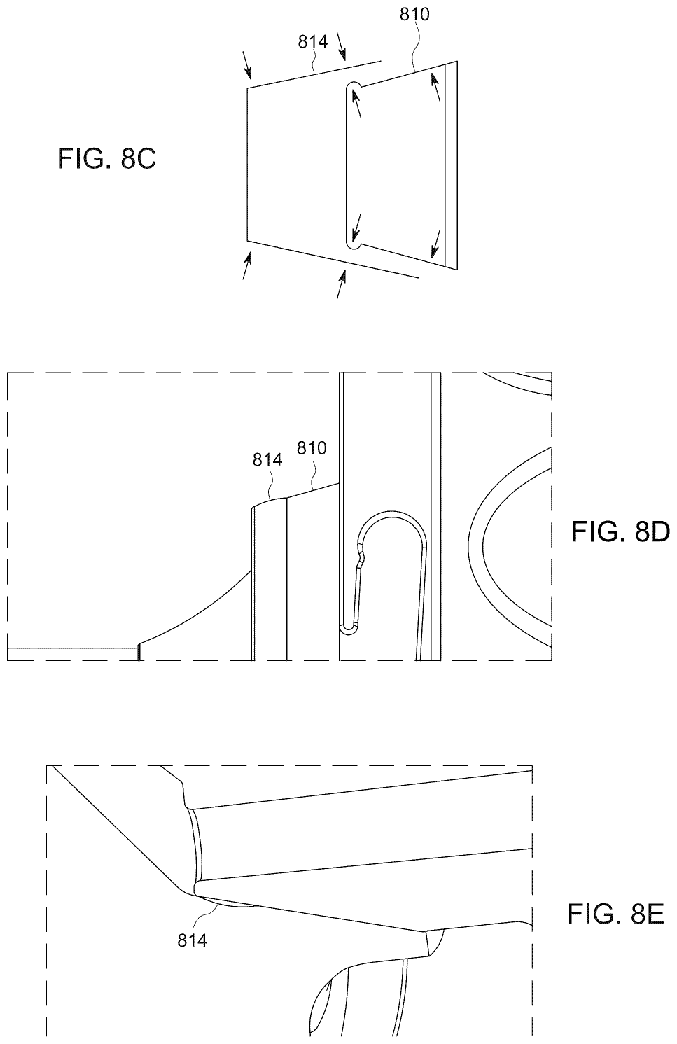

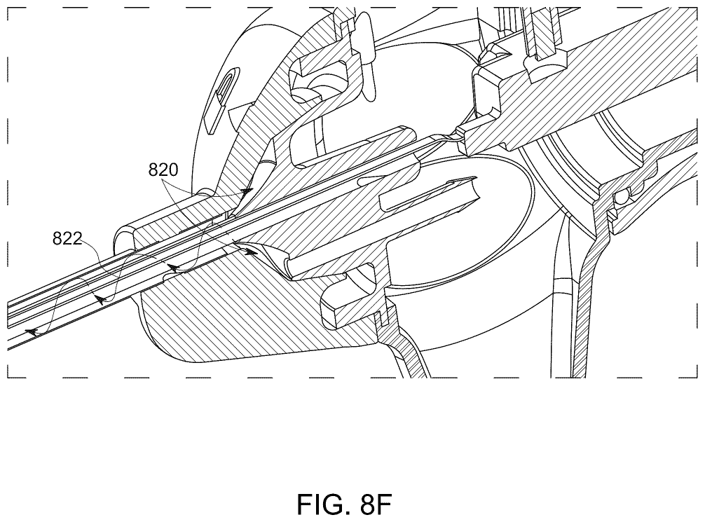

Referring to FIGS. 8A and 8B, the joint between the handle body and the outer sheath or insertion trocar 102 may be designed to provide a water tight seal between the endoscope and the outer sheath/trocar 102 without the use of an O-ring. An O-ring at this joint may be undesirable if it could fall off mid-surgery or be introduced into the patient when the endoscope is being locked into the outer sheath/trocar 102. In FIGS. 8A and 8B, the seating cup for the outer sheath/trocar 102 is shown in cutaway section, so that the male conical inner surface 810 of the seat can be seen, and the female mating cone 812 on the nose of the endoscope is shown in plan view, so that the mating between the outer surface of the male cone with the inner surface of the female cone in the cup of the outer sheath/trocar 102 can be seen.

FIG. 8C shows the two cones 810, 812 with some of the dimensions exaggerated. Male cone 810 may have a slightly sharper angle of conicity than female cone 812, by about %°. The largest diameter of female cone 812 may be slightly smaller than the largest diameter at the base of the male cone 810, so that these two surfaces 810, 812 will form a watertight interference seal. The interference of diameters may be on the order of ½ mm, when the diameters of the two cones are in the range of 19 to 25 mm. The angle may be on the order of 15°, and the two cones may differ by about ½°. Likewise, at the small end of the cones, the outer diameter of the male cone 810 may be the same as the inner diameter of the female cone 812, but male cone 810 may have small ridge 814 at its circumference, which leads to a watertight interference seal at this end as well. The polymers of the parts may be chosen to provide an appropriate amount of resilience for the interference fit to appropriately seal the water.

FIG. 8D shows ridge 814 molded onto the small end of the male cone. The ridge on male cone 810 may form an interference seal with the small end, inner diameter of female cone 814 in the mounting cup of outer sheath/trocar 102. FIG. 8E shows the two parts mated.

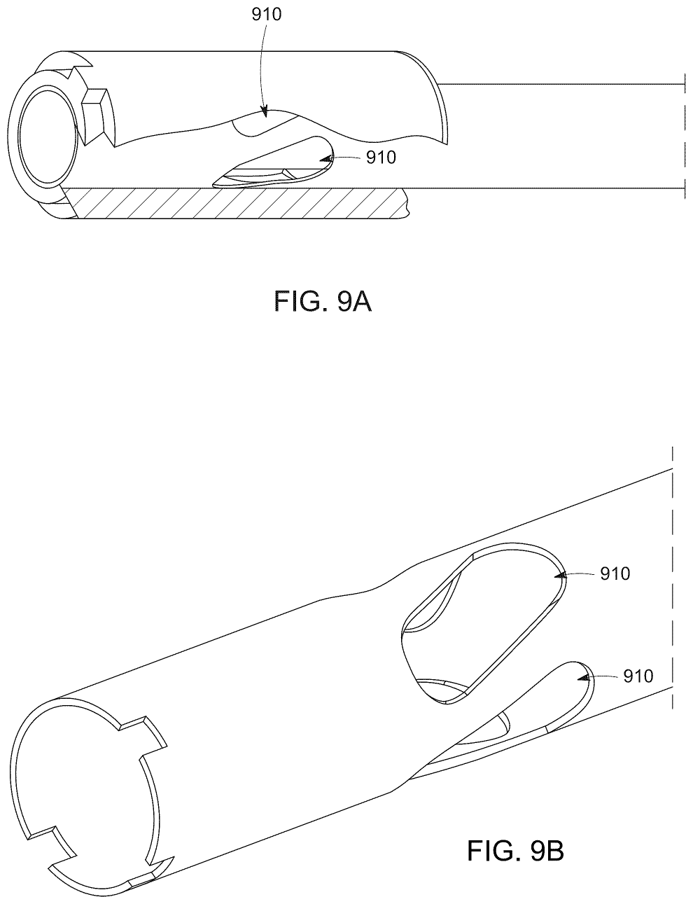

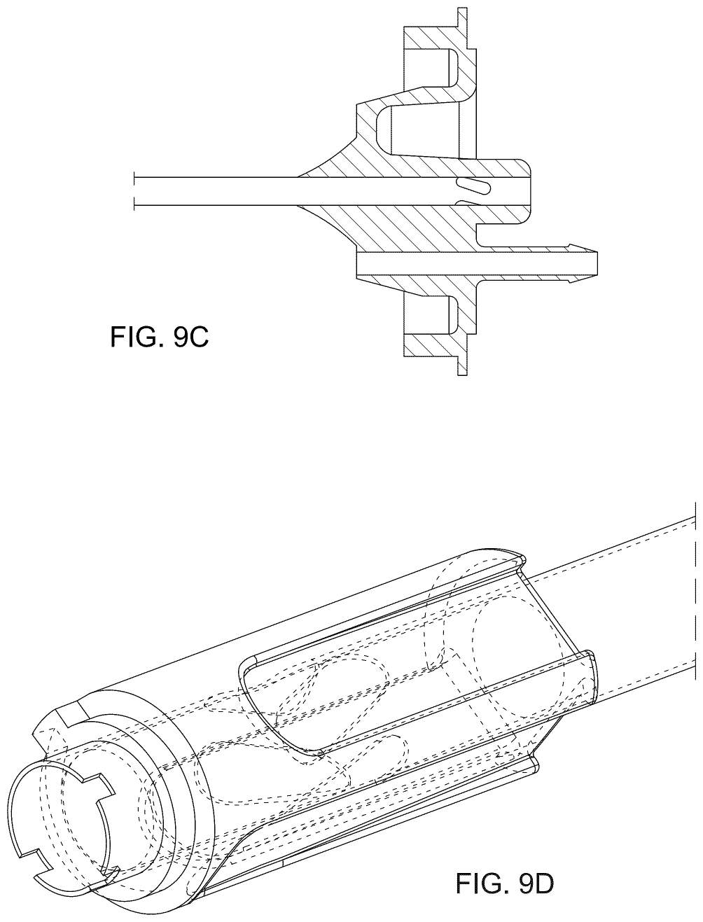

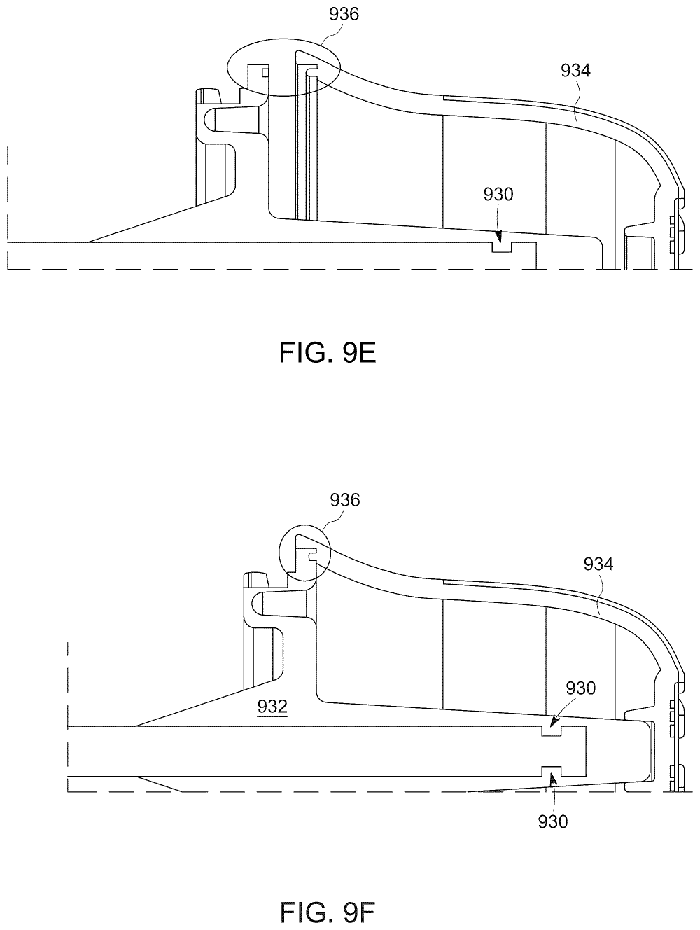

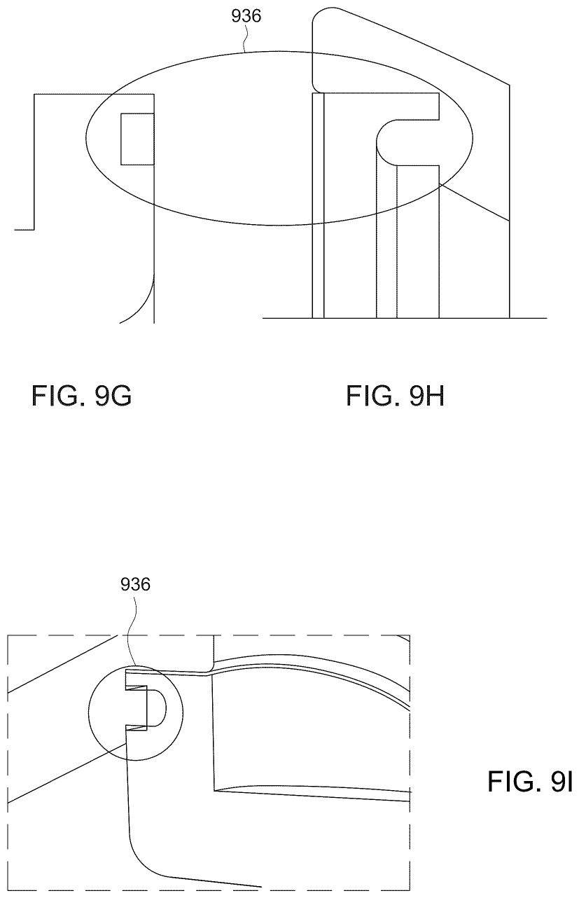

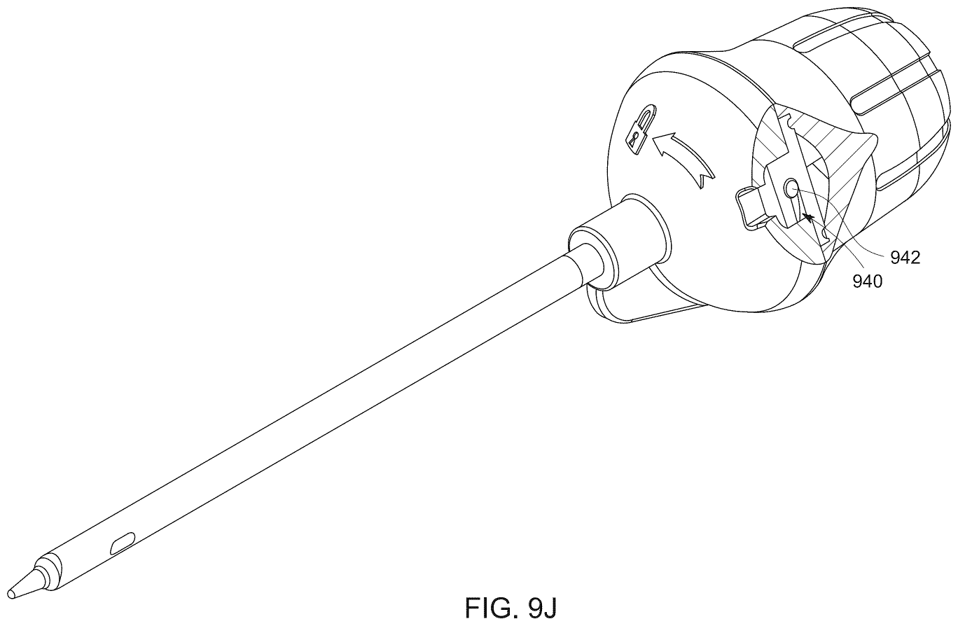

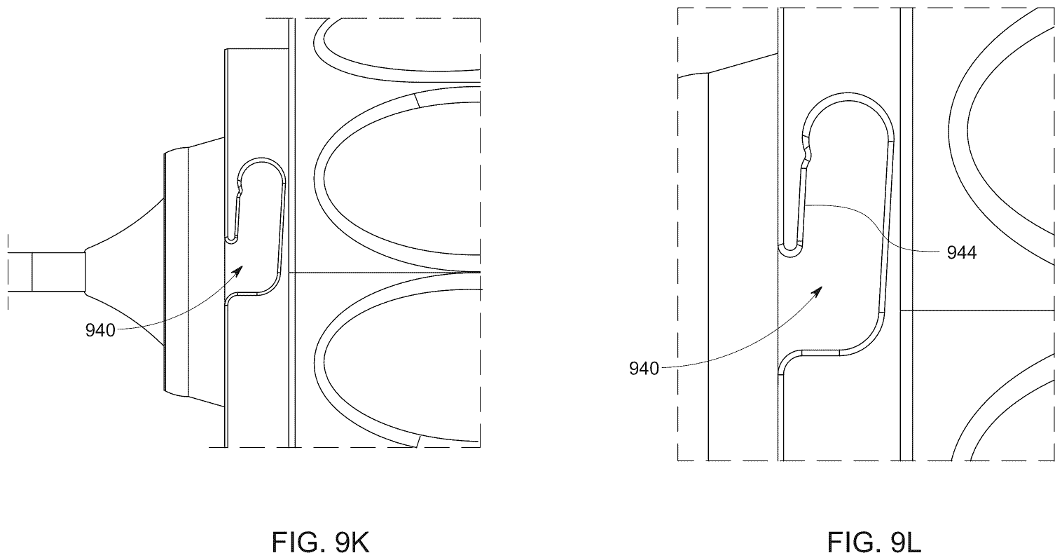

In other alternatives, the seal between the endoscope and outer sheath/trocar cap may use O-rings. O-rings may be seated in channels on the surface of the inner male cone, so that the female cone engages to compress the O-ring into its channel before the contact begins to translate into lateral forces that would displace the O-ring. O-rings may be especially desirable at the large-diameter end of the male cone abutting the face of the chassis of the endoscope, so that the female surface of the outer sheath/trocar cap cannot displace the O-ring. In some cases, the angles of the cones may be flatter than 15°, so that the compression force against the O-rings created by the twist lock (see discussion of FIGS. 9J, 9K, and 9L, in section X.C) is increased relative to lengthwise frictional force that would tend to displace the O-rings.

### IX.B. Inducing Spiral Flow

Referring to FIG. 8F, the chamber 820 between the fluid indeed tube and the fluid flow annulus of outer sheath/trocar 102 may be designed as a squeezing cone to impart spiral flow to water or similar irrigation fluid. Spiral flow 822 may be induced via the Bernoulli effect and Venturi effect as the water flows through the narrowing conical passage. Spiral vanes on the two conical surfaces may further accelerate the spiral flow.

This flow pattern may have several advantages. Spiraling flow 822 may increase pressure and water velocity as the water emerges from the flow director holes at the tip of the endoscope. Spiraling motion 822 may help clean any debris that accumulates on the front window in front of camera 410.

### X. Molding and Joining

### X.A. Diagonal Slots to Connect Dissimilar Materials

Referring to FIGS. 9A, 9B, 9C, and 9D, parts made of stainless steel may be connected to parts made of plastics such as ABS without using adhesive. One such technique is to cut holes 910 in the stainless steel and to overmold with ABS. Holes 910 may be shaped as slots, and the slots may be diagonal to the basic thrust and rotational forces. Diagonal slots 910 may provide additional securing in multiple dimensions. ABS has a shrinkage ratio, so a technique for confining play between the ABS and stainless steel may be advantageous. Joining slots 910 may be used at multiple points, for example at the base of outer sheath/trocar 102 where the base cup of outer sheath/trocar 102 mounts to it, at the base of the endoscope insertion shaft/inner sheath/cannula mounts in the nose of the endoscope, and to join the endoscope flow director onto the distal end of the insertion shaft.

### X.B. Joining Component Parts of Obturator

Referring to FIGS. 9F, 9G, 9H, and 9I, the handle/cap of obturator 104 may be hollow, to reduce weight and to reduce material costs. The shaft of obturator 104 may be made of solid stainless steel, because it is used to force a piercing access portal into the patient's tissues. As shown in FIG. 9E, a circumferential groove 930 may be lathed into the handle/proximal end of the obturator shaft, or two parallel cuts 930 may be milled. Base 932 of the obturator cap may then be overmolded onto this proximal end. Then the shell 934 of the handle may be ultrasonically welded 936 onto the base part. Advantages include reducing use of material, which reduces weight and cost. The ultrasonic welding may joint the outer shell to the inner base of the handle without small metal parts that may become dislodged into the patient, without adhesives that may be toxic.

FIG. 9H shows the male energy director, and FIG. 9G shows the female trough. After ultrasonic welding 936, the two parts are melted together, as shown in FIG. 9I.

### X.C. Twist-Locking the Parts Together

Referring to FIGS. 1C, 9J, 9K, and 9L, keyway 940 on a bayonet of the obturator handle may accept a key 942 on the inside of the mounting cap for outer sheath/trocar 102, which locks the two together during the piercing step. Slope 944 may be on the order of 3° in order to draw the parts together. Likewise, one or more keyways on a bayonet on the nose of the endoscope may engage one or more pins on the inside of the mounting cap of outer sheath/trocar 102, to lock them together during observational use of the endoscope. The key and keyways of the three components may be designed to be mutually compatible and interchangeable. This may reduce manufacturing costs, and may reduce handling during surgery.

### XI. Endoscope Tip

Components of endoscope tip 400 may be designed to permit an image sensor 410, lens, filter, an illumination emission source 418, and a window to be mounted within a confined space, such as an endoscope or an arthroscope for joint surgery, having a diameter of 6 mm or less, 5.5 mm or less, 5 mm or less, 4.5 mm or less, or 4 mm diameter or less. In some cases, fluid management may be managed in the same space. In some cases, the shaft may have the strength and rigidity commonly found in arthroscopes. In some cases, the illumination emission may be by one or more LEDs located at or near the endoscope tip. In other cases, the illumination emission may be via fiber optical fibers 430 and/or light guides 450 that conduct illumination light around the image sensor 410, within the diameter of shaft 110.

### XI.A. Molding and Assembly of Components of the Endoscope Tip

Referring to FIGS. 10A, endoscope tip 400 may be formed of spacer clip 1020 that retains flexible circuit board 416, which in turn mounts camera 410 and LED 418. Spacer clip 1020 and camera housing 1012 may be formed of an opaque biocompatible plastic such as Lustran 348. Camera 410 may be mounted on one side of flexible circuit board 416, and LED 418 on the other side. Clear window 420 may protect image sensor 410 from the outside environment, such as the tissues and bodily fluids of an endoscopic procedure, and pressurized insufflation fluids.

Referring to FIGS. 10B and 10C, components of the tip may be mounted on a flexible circuit board. Flexible circuit board 416 may be bent into channels of a brace, chassis, or spacer clip 1020 to bring the illumination emitter (an LED or emitter end of a fiber optic fiber or light guide) into place. Flex circuit board 416 may have multiple layers of printed wires on surfaces of multiple planes of the board. To provide signal integrity, shielding from interference, impedance control, and mechanical flexibility, ground planes may be laid onto the board as a hatch pattern (as opposed to a conventional solid ground plane). Layers with signal wires may be alternated between layers of hatched ground plane. Different parts of the board planes may be used for signal or ground plane, alternately, to provide desired electrical properties. Various spacing and geometric properties may be tuned and adjusted to provide desired impedance matching and signal shielding, and to improve manufacturability given manufacturing tolerances.

Referring to FIGS. 10D and 10E, the lens and filter elements may be retained in camera housing 1010. Camera housing 1010 may be molded around the lens elements. Alternatively, the lens and filter elements may be assembled, and then camera housing 1010 may then be lowered onto to image sensor, and fused together. Camera housing 1010 and lens assembly may be affixed to the terminal end of flex board 416. The affixation may be by means of adhesive, thermal welding, or acoustic welding. The lens assembly may include an IR cut filter to remove unwanted IR from entering the image sensor. The combination of flat and angled faces may be tailored to match the interior 1042 of tip outer jacket 1040 to ensure that camera 416 is located precisely in tip 400. Front flat face 1044 of the lens barrel of camera housing 1010 may be positioned to be pressed against window 420 to position camera 410.

Alternatively, the lens and filter elements may be adhered directly to the image sensor. The spacing between the image sensor and the lens and filter elements may be controlled via glass beads of a diameter matching the desired spacing.

Referring to FIGS. 10F, 10G, 10H and 10I, spacer clip 1020 may have a mounting face 1022 for camera 410, a retaining pocket 1024 for LED 418, and a curved channel 1026 into which flex board 416 slots. Mounting face 1022 may be slightly recessed to account for flex boards of varying thickness, or to permit use of a pressure sensitive adhesive. The positioning of the camera must be quite precise, and that is effected between the spring urging of flex board 416 against window 420, as described below in ¶ [0157]. Pocket 1024 may allow LED 418 to float slightly forward of its final position, so that it will be pressed against the rear surface of window 420, as described below in ¶¶ [0157] and [0158].

Referring to FIGS. 10J, 10K, and 10L, a tip may be assembled by connecting LED 418 to one side of flex board 416, and camera 410 to the other. In both cases, the electrical connections may be soldered, and the structural components may be glued. The affixation may affix two of the four sides of camera housing 410, 1010 (for example, the long sides), and leave the other two sides unaffixed. Leaving two sides unsealed may avoid trapping gas inside camera housing 1010 during the gluing process, and may provide relief for thermal expansion. Flex board 416 may be threaded into channel 1026 of spacer clip 1020. Then LED 418 and the tip of flex board 416 may be threaded through hole 1028.

Referring to FIG. 10M, LED 418 and the tip of flex board 416 may be tucked into retaining pocket 1024 so that LED 418 faces out.

Referring to FIGS. 10A and ION, shaft 110 may be inserted into the plastic flow director at the end of trocar. Insertion portion 1036 of spacer clip 1020 may have an asymmetric octagonal shape to engage with a mating asymmetric octagonal opening of the plastic flow director. The asymmetric shape (or keying) ensures proper orientation. The flow director may have a tongue 1032 and spacer clip 1020 may have a mating recess 1034 that lock together to ensure that the two parts are assembled in proper orientation to each other and to resist twisting in user.

Referring to FIG. 10O, tip outer jacket 1040 with transparent window 420 may be slid over spacer sheath. Spacer clip 1020 may have a profile (such as a trapezoid) that keys with a mating profile of a hole 1042 of tip outer jacket 1040 to ensure only a single assembly orientation.

Referring to FIG. 10P, the flexibility of board 416 may tend to urge camera 410 forward against window 420 at flush contact 1044, and may urge LED 418 forward against window 420 at flush contact 1045. Tip outer jacket 1040 may have interior features 1042 that engage with the face of camera housing 410, 1010 and the face of LED 418 to retain camera 410 and LED 418 in precise orientation. For example, the beveled corners of camera housing 1010 may mate with beveled internal features 1042 of tip jacket 1040, to ensure that the camera is positioned precisely with respect to the tip jacket 1040.

Resilience of flex board 416 through the bends of channel 1026 and the rear face of window 420 may urge LED 418 and the flat face surfaces 1044 of camera housing 1010 against the interior face of window 420, which holds LED 418 and camera 410 in precise angular alignment. This may tend to hold camera 410 precisely perpendicular to window 420, to reduce refractive distortion. LED 418 may have a light distribution cone 1046 of about 30°. At the outer surface of window 420, a few percent of the light may reflect 1047 back into the interior of the scope. The spacing between LED 416 and camera aperture 1048 may be large enough that the back-reflection 1047 does not enter camera aperture 1048.

Referring again to FIG. 10A, spacer clip 1020 holds LED 418 at a position forward of camera 410 lens. Because most of the light from LED is emitted in a forward direction, keeping the camera back of this light emission cone (1046 of FIG. 10P) may reduce light leakage into camera 410.

Referring to FIG. 10Q, the components at this point may be designed to engage with each other sufficiently via slip/press fit to maintain integrity without adhesive. Outer jacket 1040 may be designed to precisely fit over spacer clip 1020, so that outer jacket 1040 may be very thin to work within the very confined space available inside tip 400, while the combination of outer jacket 1040, spacer clip 1020, and flex board 416 may fit together closely to provide structurally integrity. The fit may leave a trough 1049. An adhesive, such as an ultraviolet-cure adhesive, may be laid into trough 1049 via a needle, as shaft assembly 120-is rotated. This adhesive may be cured to seal against fluid intrusion and provide a final structural lock.

This overall design philosophy may permit reconfiguration and reuse of much of the engineering for endoscopes of varying size, scalable depending on how small the sensor is and the need of the specific surgery (in contrast, for rod-lens scopes, many design decisions are specific to a single design). Features that contribute to scalability include the use of a single flex board, the top and bottom brace or chassis 412, 414, 438, and overmolded window 420. Poka-yoke design principles may be applied to ensure that each assembly step permits only one orientation.

### XII. Image Processing Unit

Referring to FIGS. 11A and 11B, the image processing unit (IPU) may use an interface board to drive and receive signals from the scope via the cable and a custom or off-the-shelf motherboard. In some cases, the motherboard may be an off-the-shelf motherboard with an Intel CPU and an Nvidia GPU. The motherboard provides most of the external interface ports. The patient may be isolated from the line voltage (110 or 120V 60 Hz in the U.S., 240V 50 Hz in Europe) by a medical grade AC/DC power supply and a separate interface board, called the patient interface board The patient interface board processes signals to convert them between signal forms used internally to the IPU and the signal forms that travel to and from the scope.

### XII.A. Image Processing

An image processing computer may perform image processing. GPUs provide a well-documented API that can be exploited for acceleration of graphical processing, and the software running on the motherboard may in turn have internal APIs that permit combining software processing components for image enhancement. A series of video chips in the scope handle and the IPU (Image Processing Unit) box may convert the very small, high speed video signals from the sensor (such as a Bayer formatted MIPI-CSI2 interface) to a signal suited for transmission distances longer than a few centimeters, and to a protocol more easily processed by various stages of an imaging pipeline and for storage (such as YCbCr422 or MPEG). The IPU processor may receive data from the scope (which may be video data, still images, telemetric data, etc.) via the handle board, cable, and patient interface board. The IPU may capture still images out of the video, and/or process the video through image correction and enhancement software to deliver an high-quality image on the monitor or for storage on some storage medium or in the patient record.

Various video signal processing chips, an image signal processor (ISP) and a graphics processing unit (GPU) may perform a number of video transformations on the video data received from the scope before the data are displayed on the monitor or saved to an output device. The IPU box may have multiple processors, including a specialized image signal processor (ISP), a general purpose CPU such as an Intel Pentium, a graphics accelerator (GPU), a field programmable gate array (FPGA), a custom accelerator hardware, and perhaps others. Video transformations may be performed in one or another of these processors, or in software, or some combination of hardware and software. The sum total of processing power may be chosen to ensure that the image processing may be performed within requirements for image latency. The following transforms may be performed:
Receive raw image data from the image sensor of the endoscope in a Bayer Formatted MIPI-CSI2 stream and re-encode to a YCbCr422 or h.264 MPEG stream to improve processability
Translate a MIPI-CSI2 video stream into a UVC complaint USB 3.0 video stream via a video stream processor such as the Cypress CX3.
HDR or WDR processing (High Dynamic range or Wide Dynamic range)-software (a) to expand the dynamic range of the captured image by avoiding over or under-exposed areas of the video. This is achieved by combining sequential over and under exposed frames of images from the image sensor and reducing the displayed intensity of exceptionally bright pixels to reduce hot-spotting, and increasing the displayed intensity of exceptionally dim pixels in a frame to improve visibility images. See FIG. 11F. HDR/WDR processing may use the Mertens exposure fusion algorithm.
Rotate and image righting based on the handle's rotation sensor (see discussion of FIGS. 3C, 3D, and 3E, at ¶ [0067]). Including the display of and rotation of a position indicator, which may be displayed as an arrow, around the perimeter of the circular mask on the user interface.
Correction of distortion (either systemic because of fish-eye distortion or similar distortion in the lens specification, or specific distortions measured in specific scopes to be corrected in the IPU by a reverse transform), removal of artifacts.
Crop the rectangular image received from the scope to a rectangle that can be rotated around a central point in the display. A circular mask is applied over this rectangular crop to provide a circular image display to the user. This may replicate the view surgeons are used to from decades of rod lens scopes. Also, outside the field of view cone provided by the lens, the outer edges of the image may be so distorted or obscured by the edges of the lens housing, that it communicates more distraction than information.
Auto-exposure to target a desired average image brightness by adjusting exposure times and gains in the image capture pipeline.
De-mosaic
Black Level Correction
Gain Adjustment
Shading Correction
Defect Correction
Noise Reduction
Tone Mapping
Color correction and white balance correction
Zoom in/zoom out within the target image
Lens resolution correction
Local Contrast Enhancement
Edge Enhancement
Image enlargement (enlarge the circle displayed on the monitor, perhaps losing the upper and lower limb of the circular display)
Reformatting and compressing the video data for storage on a storage device, and decompressing stored video data for display.
Controlling transmission over network connections for storage in the cloud or on storage devices local to the IPU, or other non-cloud storage
Super-Resolution is discussed below in § XII.D at ¶¶ [0173] to [0182]—this upsamples from a lower resolution (for example 1280×720) resolution to 2160×2160 (“4K”) resolution
Frame Writer is the last stage, putting the video into the video system's frame buffer for display or to storage. The fully-processed video stream may be displayed on a video monitor, or may be sent to a storage device or network interface.

Dividing the pipeline into phases allows parallelism. For example, each phase may be assigned to one core of a multi-core CPU or different functional units of a GPU.

### XII.B. HDR Exposure Fusion to Preserve Frame Rate

Referring to FIG. 11F, HDR exposure fusion may be performed on pairs of frames taken simultaneously by two different cameras, and then the images are merged pairwise. Exposure fusion algorithms include Mertens-Kautz-Van Reeth, or Hugin/Enfuse.

In other cases, a single image sensor may be programmed to overexpose frame n, then underexpose frame n+1, then overexpose frame n+2, etc. This may be controlled by strobing illumination LED 418 at the frame rate, or by controlling the exposure time of the image sensor. The short exposure time frames may bring out detail in overexposed parts (“hot spots”) if the image, and the overexposed frames may bring out detail in underexposed parts of the image (“dark areas”). By merging the frames, both hot spots and dark areas are captured in the output image, increasing the dynamic range that can be captured.

The frames may then be merged pairwise using HDR exposure fusion algorithms of the same class, except applied to overlapping pairs of frames, to merge frame n with frame n+1, then merge frame n+1 with frame n+2, then merge frame n+2 merged with frame n+3, etc. This maintains the output frame rate at the input frame rate.

### XII.C. Auto Exposure

An auto exposure algorithm may be used to adjust for fluctuations in light intensity level of a scene the image sensor is capturing to a target brightness level. If the camera is moved close to an object with static gain, exposure, and illumination intensity, the overall scene becomes brighter and therefore the exposure times, gain, and/or illumination intensity per frame should be reduced to capture less light. Conversely, if the camera moves farther away from an object, the overall scene becomes darker and exposure times, gain, and/or illumination intensity should be increased to capture more light.

An auto exposure implementation may control both the exposure time and gain to achieve a target intensity setpoint. The gain control may be either analog gain in the cell of the pixel of the image sensor, or digital gain applied in the image sensor or digital image processing pipeline. The brightness setpoint may be set via a user “brightness” control, or may be set automatically. The auto exposure algorithm may perform the following steps:
1. Divide the frame into n×n-pixel blocks.
2. Compute the average intensity for each block.
3. Compare the computed intensity for each block to an intensity setpoint (which may be set for each block, or for the image as a whole) to get an error value for each block. Each block may be assigned a weight to scale its computed error value. This weight allows for certain blocks to be more important than others (i.e., blocks in the middle of the grid weighted higher than those further out).
4. Sum all weighted block errors for an overall error value.
5. Evaluate the change:
   a. If the overall error value is below the defined change threshold, no changes are made.
   b. If the overall error value is above the defined change threshold, scale the change for one update cycle, update change threshold relative to a size of the overall error by the equation below.

Max Change Threshold=Max Change Threshold+(Overall Error×Multiplier)
where Multiplier is less than 1 to allow a damped response.
c. The max threshold is set to minimize the perception of a discrete light level change by the user in similar use environments, but allow fast updates when quickly changing from dark to light or light to dark environments. The multiplier is used to tune this response to achieve the fastest response time to large changes in environmental conditions while preventing oscillations in the light levels perceived by the user.
6. Input overall error into either the exposure or gain PID control:
   a. If the scene is too bright:
      (i) If the gain is at its minimum, the exposure PID control runs
      (ii) Otherwise, the gain PID control runs
   b. If the scene is too dark:
      (i) If the exposure is maxed out, the gain PID control runs.
      (ii) Otherwise, the exposure PID control runs
   c. Depending on implementation, any two or more parameters may be substituted for gain and exposure, including illumination intensity, exposure time, etc.
7. Write resulting exposure and gain to ISP.

The auto exposure algorithm may be downstream from the WDR algorithm, perhaps the immediately following stage. This reduces sensitivity of the auto exposure algorithm to frame to frame changes in exposure time used by the WDR algorithm. The auto exposure algorithm may run every several frames (rather than every frame) to reduce processing bandwidth. The per-block intensity computation may be parallelized to run on the GPU.

Software may provide that many of the parameters of this algorithm may be tunable via a config file loaded as part of system startup, including the number of frames allowed to run between recalculation of the autoexposure parameters, the block size for step 1, the mean intensity setpoint of step 3, a map of block weights for step 3, the PID coefficients for the PID calculation of Step 5.

XII.D. Video processing for superresolution

Referring to FIGS. 11C and 11D, the input to the Super Resolution block may be low resolution video (for example, 720×720 pixel (“720p”) or 1280×720 image, and the output may be an enhanced quality 2160×2160 pixel (“4K”) image. The “Super Resolution” box may in turn have a block diagram as shown in FIG. 11D. A machine learning model may be used to combine noise reduction, lens resolution correction, edge enhancement, local contrast enhancement, and upscaling as an integrated module. When these functions are performed singly, each is subject to various tradeoffs, and image enhancements by one stage may interfere with and degrade enhancements from another stage. For example, many noise reduction algorithms tend to result in blurred images. Traditional edge sharpening tends to amplify noise. By combining all these functions in a single machine learning model, those tradeoffs may be reduced.

Various types of machine learning models can be used with the systems disclosed with respect to FIGS. 11C and 11D, including fully convolutional neural networks, generative adversarial networks, recurrent generative adversarial networks, or deep convolutional networks. Convolutional neural networks (CNNs) are particularly useful for image processing. The Super Resolution CNN block may be formed by combining:
A CNN upscaling module from NexOptic Technology Corp. of Vancouver, B.C. This may allow a processor to infer inter-pixel interpolations based on local information, and previous and next frame information, to improve apparent resolution.
A noise reduction module from NexOptic. This may reduce noise from the image sensor, electronics, and stray light photons
A lens resolution correction module from NexOptic. This step may enhance the performance of the lens by understanding the transfer function of a fixed image through the lens.
A local contrast enhancement module from NexOptic. This may assist the surgeon by increasing contrast between light and dark, various shades of red, etc.
Dynamic range compensation-portions of the image that are washed out because of overexposure may be balanced out against parts of the image that are washed out because of darkness. The total dynamic range may be adjusted to improve contrast and to draw out detail that is lost in the over- or under-exposed portions (see FIG. 11F).
An edge enhancement module from NexOptic. This may reduces loss of resolution (blurring) that may have been introduced by the lens system (e.g., due to limitations of lens size or complexity) or by motion of the camera of objects in the scene, and may improve edge extraction to assist a surgeon by making structures more apparent at the surgical site.
High entropy random noise interferes with data compression. The CNN may be trained to recognize and remove random pixel noise, which may improve data compression.

By combining all these functions into a single CNN, local contrast, edge enhancement, and noise reduction may all be simultaneously improved. Much like human neural networks skillfully optimize for multiple parameters simultaneously, a computer CNN may be trained to simultaneously optimize for several characteristics. Hardware contrast and edge enhancement may be disabled. In some cases, the degradation and training may involve at least two of the parameters in above list, for example, resolution and edge enhancement, or resolution and local contrast. In some cases, any three of these types of image degradation may be trained into the model, for example, resolution, local contrast, and edge enhancement, or resolution, image sensor noise, and lens correction. In some cases, the model may be trained on any four of these parameters. In some cases, it may be trained for all five.

In one example implementation, given an input sequence of low resolution frames {I_{-T}^{L}, . . . , I₀^{L}, . . . , I_{T}^{L}] a sequence of high resolution frames Iᵢ corresponding to the corresponding to the low resolution frames. The super resolution frames may be computed

1: For all i except the reference frame
2: Compute warping Fᵢ^{T} from S^{T} Iᵢ^{L} to S^{T} I₀^{L}
3: Compute Yᵢ = Fᵢ^{T} S^{T} Iᵢ^{L}
4: End
$5: Compute Q = {\sum }_{i = - T}^{T} {F}_{i}^{T} {S}^{T} {SF}_{i}$
$6: Compute SR draft Z = {\sum }_{i = - T}^{T} {Q}^{- 1} {Y}_{i} .$

where
T is the radius of the temporal neighborhood
F is the warping operator for frame i to the current frame
Sᵢ is the decimation for frame i

The video super resolution model may execute in two steps: a motion estimation and compensation procedure followed by an upsampling process. Alternatively, instead of explicitly computing and compensating for motion between input frames, the motion information may be implicitly utilized to generate dynamic upsampling filters, and the super resolution frames may be directly constructed by local filtering to a frame being constructed in the center of a computation window. The machine learning model may be trained by capturing reference video at normal resolution, and then degrading the reference video via transforms that simulate loss of resolution, introduction of noise, lens aberration and similar lens noise, degrading contrast, and/or degrading edges. The machine learning model may be trained to recover the full resolution original reference video. That same training may be sufficient to allow video captured at normal resolution to be upsampled to higher resolution. A lens model may be created from a combination of design data and images captured of standard test patterns (for instance a checkerboard or array of Cartesian lines) to detect and measure lens imperfections for a lens design or specific to each scope, and create a generalized transform or store registration correction for a specific scope. In some cases, high quality reference data may be displayed on a physical display, and viewed via an endoscope camera. The machine learning model may be trained to recreate the reference data from the camera video. The training may exploit the [symbol] 1 loss with total variation (TV) regularization to reduce visual artifacts

The lens correction model may address the imperfections in a lens system that remain after balancing for all constraints, for example, by creating a lens model and passing a large set of ultra-high resolution images captured with a camera with a very high quality lens (to establish a baseline “perfect” image) through the lens model, then training the CNN to correct that the image set passed through the lens model to transform each image into the “perfect” image.

The Super Resolution CNN may yield better overall image quality (compared to the raw data directly out of the camera, and compared to using all classical blocks independently). Combining classical enhancement algorithms with the enhancement CNN may provide opportunities to tune parameters of the classical algorithms in parallel based on the CNN training where classical algorithms require tuning parameters in series. The Super Resolution CNN may allow tunable runtime performance via architecture choice allowing for tradeoffs between overall image quality and speed.

In some cases, the CNN may retrain itself on the fly. For example, at moments when the camera and image are stationary relative to each other, alternating frames may be taken at deliberately underexposed (too dark) illumination and normal illumination. The CNN may be retrained to recognize hot spots where detail is lost because of overexposure, and where detail is lost in the dark regions of the underexposed frame. In some cases, several machine learning systems may be chained together, for example, one to enhance dynamic range, one to reduce blur and for edge sharpening, one to recognize frame-to-frame motion, one to improve contrast, and one to upsample for super resolution.

In some cases, a bypass feature may disable the Super Resolution neural network, and instead upsample the image to 2160×2160 resolution via conventional means such as bicubic interpolation.

The NexOptic components may be obtained under the product name Super Resolution, as described in U.S. Pat. No. 11,076,103, Gordon, Photographic Underexposure Correction Using a Neural Network, and U.S. Publication No. 2021/0337098 A1, Gordon, Neural Network Supported Camera Image or Video Processing Pipelines, both incorporated by reference.

### XII.E. Diagnosis and Lesion Detection

In some cases, the image processing pipeline of FIG. 11B may include processing to detect various lesions. For example, during colonoscopy, the image processing pipeline may have a processor to detect polyps. During esophageoscopy, the image processing pipeline may have a processor to detect Barrett's esophagus.

### XII.F. Scope Control

The scope may have several controls, including a pushbutton on the scope, a touch screen on the face of the IPU, and a graphical user interface with a touchscreen that may be accessed over the internet from an external computer.

One pushbutton on the scope may control three things: (a) still frame capture, (b) video record on/off, (c) LED adjustment, high beam/low beam. For example, one press may capture the current view as a still frame. A doublepress may start or stop the video recording. A triplepress or a press for three seconds may adjust the LED brightness.

The IPU may have front panel controls for the scope, including image adjustment, color, brightness, zoom, and the like. In either a user-visible or a system set-up/testing mode, controls on the front panel of the IPU or accessible via a computer over the internet may control:
LED illumination—because the on-scope button is only a single momentary connection switch, it cannot provide fine control, only gross on/off control. Another user interface may provide finer lighting control
Sensor control—adjust tone or color balance, zoom, etc.
Control image and video storage in the IPU's nonvolatile memory—which portion of which video to store, etc.

Adjustment of LED brightness requires careful integration with the image sensor. If brightness is controlled by conventional pulse width modulation (PWM) that is not synchronized with the frame sync of the image sensor, banding can occur in the image. Alternatively, a constant current source or voltage controlled current source may be used to adjust the LED brightness and avoid banding.

### XII.G. Flexboard and Electronics in the Endoscope Handle

Flex circuit board 416 may carry signal and power from the handle to the components at the tip. At the tip, molded plastic parts (brace or chassis 412, 414, 438) may hold all the component parts in proper orientation. The components (image sensor, lens, filter, window, and mounting) may be selected to assure a desired offset angle (typically 0° on-axis, 30°, 45°, or 60°) and a desired field of view (typically 50°, 60°, 70°, 80°, 90°, 100°, 130°, or 180°).

The distance from the image sensor at the tip to the receiver on the circuit board in the handle may be about 115 mm to 330 mm, relatively long for a MIPI-CSI2 video connection. The flex circuit board may have circuit layout and shielding chosen to create an impedance matched signal path for the video data from the video sensor, with low radiated emissions, low loss, and low sensitivity to external interference. A connection from the inner insertion shaft to the handle circuit board's isolated reference potential may protect against interference from RF ablation or coagulation devices by allowing the video signals from the image sensor to float relative to the RF application energy, minimizing the interference induced on the signal conductors transporting the MIPI-CSI2 signaling from the image sensor to the handle board.

A rigid circuit board in the handle (HB PCBA—“handle board printed circuit board assembly”) may have a microprocessor, magnetic sensors, and a transmitter chip. The transmitter chip may receive the low-power, high-bandwidth, high speed signals, which may be transported using a MIPI-CSI2 stream, from the image sensor received over the flexboard, and convert the video signals into serialized signals suitable for transmission over a 3-meter cable to the IPU. Because 3 meters is a relatively long distance, the cable may be carefully impedance matched with low insertion loss to ensure signal integrity. The serialized signals are received on the IPU, converted back into a MIPI-CSI2 interface, and passed to the image signal processor (ISP) for processing.

### XII.H. Cable

Referring to FIGS. 11E and 11F, the IPU may be connected to the scope via a custom cable. The cable may be about 3 meters (10 feet) long-long enough to give the surgeon freedom of movement, and to keep the nonsterile IPU acceptably distant from the patient. The connector may be customized to ensure that the scope cannot be connected to other devices that would not supply the necessary patient isolation.

The cable may use a USB Type A or C connector, because the connector has good shielding and physical insertion characteristics, even though in this application, the cable does not carry USB signals or utilize the USB protocols. The cable may have a protective hood that extends several millimeters beyond the end of the USB connector (alternatively, the USB connector may be recessed below the end of the hood). The hood may provide insulation around the connector when the cable is disconnected from the IPU, which provides the creepage and clearance distances required for electrical isolation of the patient, for example, if the end of the cable should happen to touch something electrically live or earthed. The hood may be keyed so it will only connect to the correct port on IPU, and won't (easily) plug to a generic USB connector, and ensures right-way-only connection of the cable to the connector on the IPU. The cable end and plug on the IPU box may be color coded to each other.

The cable may supply power to the scope, communicate command signals to the scope, obtain configuration information that was stored in the scope's on-board memory, and carry video signals back from the scope back to the IPU. The cable may also support a scheme for detecting that a scope is connected to the IPU. This is achieved by sensing a voltage change on a pin of the scope cable, which is pulled to a logic-high voltage when the cable is disconnected and forced to a logic-low when the cable is connected. A pin on the cable may be connected to a pull-up resistor on the IPU side and pulled to GND on the handle board side, so when the handpiece is connected to the IPU, the handle board pulls the pin down and a processor may detect that a handpiece is connected.

### XII.I. Wireless Communication in Place of Cable

The cable connection between the IPU and the handpiece may be replaced by wireless transmission such as Bluetooth, Wi-Fi, or some other wireless protocol. In these cases, the handpiece may have a battery whose capacity can drive the handpiece for the longest length of a surgery. A wireless connection may provide an alternative architecture to implement electrical isolation of the patient, as required by the IEC 60601-1 standard.

### XII.J. Isolation

Referring to FIG. 11G, the patient interface board may electrically isolate the motherboard from the patient-facing cable and scope by providing an optical connection or transformer to interrupt the copper signal path. The isolation of the data may be provided between the video stream processor (such as the Cypress CX3) and the motherboard via a fiber optic cable driven by USB 3.0 transceivers on each end of the cable, without power conductors, that allow an interruption of copper conductors, while communicating via the USB 3.0 communication protocol.

The physical interface between the scope and the IPU may be a USB 3.0 cable consisting of three twisted pairs, a ground conductor, and a power pair of wires, although the physical layer communication is not USB 3.0. The patient interface board may electrically isolate the processing circuitry from the patient-facing cable and scope by providing an optical connection or transformer to interrupt the copper signal path. The isolation mechanism may isolate the patient from the possibility of electric shock and prevent excessive leakage currents.

The IPU box may include a transformer 1170 that steps down 120/220V AC voltage to a secondary voltage used internally to operate the processing circuitry 1172 of the IPU box, and a second transformer 1180 may isolate the secondary circuitry 1172 from the patient and patient-facing circuitry.

Two safety capacitors 1182 and 1184 may be provided in series across the primary and secondary of the isolation transformer. The purpose of capacitors 1182 and 1184 is to create a current divider for common mode current created in the isolated switching supply that utilizes the transformer 1180. The lower impedance of these capacitors, relative to the parasitic capacitance between the patient isolated island 1174, including the scope, and the earth, may attract the majority of the common mode current reducing the common mode currents that travel between the patient isolation island 1174, including the scope, and earth, thereby to reduce radiated emissions. The two capacitors may be surface mount ceramic capacitors, to minimize their impedance at higher frequencies. Capacitor 1186 may be placed differentially across the secondary of transformer 1180 creating a low impedance at high frequencies across the secondary of the transformer. This low-impedance allows common mode currents traveling on the positive output of the transformer to travel to the negative output of the transformer, through capacitor 1186 and back across the transformer through capacitors 1182 and 1184. The two capacitors 1182 and 1184 may be placed in series and may be UL listed safety capacitors to comply with the requirements of IEC 60601.

A second pair of two capacitors in series 1192, 1194 may connect the USB connector shell (the metal shielding jacket on the female side of a USB connector) to two mounting holes which tie to the earth connected IPU chassis to provide a short return path to earth for common mode currents injected into the scope and/or scope cable. The capacitor pairs 1192, 1194 may be placed symmetrically on each side of the USB connector shell, both connecting to a chassis mounting point that is earth connected (for example, to the housing 1196 of IPU 1100), to improve the shielding effectiveness to common mode currents injected onto the scope cable.

The value of capacitors 1182, 1184, 1186, 1188, 1192, 1194 is selected to provide sufficient reduction of common mode currents and comply with the leakage requirements for IEC 60601-1.

A fiber-only optical cable may be utilized to transport high speed video data from the patient isolated circuits 1174 to the secondary circuits 1172 in compliance with the IEC 60601-1 patient isolation requirements. The fiber optic cable may contain USB 3.0 transceivers on each end of the cable. The high-speed video from the scope may be translated from a MIPI-CSI2 protocol used by the image sensor to a USB 3.0 protocol through an integrated circuit. The USB 3.0 superspeed RX and TX data pairs may be converted to optical signals transported over the optical cable via optical transceivers. The optical transceivers on each end of the cable may be powered locally to avoid the need to run power, and copper wires, over the optical cable allowing the cable to maintain compliance with IEC 60601-1 isolation requirements.

The patient interface board may provide the scope interface, including the BF type patient isolation required per the isolation diagram and IEC 60601-1. This includes the isolated power supply, and isolation of any other interfaces with copper wire that may conduct electricity (USB interfaces, etc.).

### XII.K. Other Peripherals

### XII.K.1. Monitor

The IPU may drive a video monitor so that the surgeon can have a real-time display of the surgery.

### XII.K.2. USB Port

A USB port may be provided on the front of the unit for use with a USB flash drive, which may be cabled to the motherboard. Four USB ports may be provided on the rear of the unit for use with a USB mouse and keyboard. Ethernet and Wi-Fi interfaces may be provided from the motherboard for network connectivity to cloud storage (see §§ XIII.C and XIII.D, ¶¶ [0228] to [0235], below). An analog microphone input may be provided on the rear of the unit as well as a Bluetooth interface that can be used for annotating during procedures. A speaker may be provided in the IPU. An AC mains plug may provide power for the IPU. The AC mains may be controlled by a power switch.

### XII.K.3. Connections to Cloud Storage

The IPU and programming may allow videos, images, metadata, and other data to be captured and saved. Programs on the IPU may allow update of software of the IPU. This data may be uploaded or backed up, for example over Wi-Fi, Bluetooth, or a similar wireless connection to the cloud, or may be stored to an external removable USB flash drive connected at the USB port. This flash drive may then be used to transfer the data to patient records as needed by the facility or uploaded to cloud storage from an external PC (see §§ XIII.C and XIII.D, ¶¶ [0228] to [0235], below).

Video may be stored in two-minute increments. If there's a write error, the length of video lost may be kept to that limit. The stored video and still images may be annotated with date, time, and location metadata, and the serial number of scope and IPU. In the cloud, the serial number may be used to connect the video and images to the right patient's medical record.

At end of each surgical day, data for the day's cases may be stored either in a cloud server or on the USB drive. If connections to the cloud fail, the USB storage may provide an easily-accessed backup. The surgeon may later access the cloud storage or USB data to transfer into the patient's medical record, and annotate with physician's notes.

### XII.K.4. USB Connection for Keyboard and Mouse

During normal operation, the scope pushbutton is the only user input available. A USB keyboard and mouse may be connected to the system to perform system configuration. A keyboard and mouse may allow entry to a service or configuration screen.

### XII.K.5. Microphones

The IPU may have a connector for a wired microphone and may allow the connection of a Wireless microphone. This may allow real-time annotation of videos captured by the surgeon. The system setup may allow the user to specify if they wish audio to be enabled and then to either connect a microphone with a 3.5 mm jack or a Bluetooth Interface.

### XII.K.6. Insufflation Tubing

Referring to FIG. 1J, for irrigation or inflation (insufflation), the scope may include a short pigtail of irrigation tubing terminating in a three way stop cock allowing the user to connect an external irrigation pump and vacuum. The pigtail may also include a line clamp. The endoscope may be packaged with a disposable, single-use tube set, with a proximal end that connects to a source of fluid such as normal saline, and the distal end having a luer lock. The tube set may have a pinch proof clear tube with a stopcock valve to select inflow or suction, and a tube clamp that can be used to stop irrigation at the scope. The clear tube supports flow through the scope's handle to the front molding of the scope where fluid passes through to the cannula cap between the cannula tube and the inner tube of the insertion shaft. The clear tube is secured to the scope front molding by using a barb fitting and a retaining clip.

### XIII. Machine-Readable Serial Number

### XIII.A. Machine-Readable Serial Number

Each scope as shipped may have one or more scope-specific data encoded in machine-readable and scannable, and/or human-readable form. The data may include one or more of the scope's serial number, configuration data, manufacturing calibration data, tracking data, etc. These data may be used for multiple purposes.

Information may be encoded in a machine-readable code on packaging for the scope, embedded in packaging, or embedded in the scope as a scannable code. The scannable code may be any form of Matrix (2D) or linear bar or machine vision code that can be scanned by a smartphone. Examples include any variant of QR code, Code 39, Code 49, Code 93, Code 128, Aztec code, Han Xin Barcode, Data Matrix code, JAB Code, MaxiCode, PDF417 code, SPARQCode, and others. The scannable code may be an RFID or similar tag that can be scanned by a sensor in a phone. The scan may be optical, or may use any IEEE 802 or related communications protocol, including Bluetooth, RFID (ISO 14443) or NFC (ISO 18092). The scannable code may be coded on packaging, in the scope's handle, or in the nose cap of a replaceable scope insertion tip. Alternatively, it may be stored in an EEPROM memory in the handset, connected by an SPI (Serial Peripheral Interface), I2C (Inter-Integrated Circuit), USB, or a one-wire protocol, to be read when the scope is plugged into the image processing unit (IPU). The scope may have a small amount of non-volatile memory that can be read and written during initial device manufacture and by the IPU. That memory may store an electronically-readable serial number written into the memory during manufacture. This memory may also store per-scope configuration information, such as scope model, serial number, white balance coefficients, lens properties that can be corrected in the IPU, focus parameters, etc. This memory may also be used to store usage information such as timestamps or usage time as determined by the IPU, to prevent reuse of the scope after 24 hours. To ensure tamper resistance, information written into the handle memory may be written under a secure or encrypted protocol used between the IPU and the handle's microprocessor.

The information may be stored as s single datum (essentially a serial number, or some other datum that semantically-equivalently uniquely identifies the scope), which may be used as an index key it a database at a server, which in turn has the full data about the scope. In some cases, various operating parameters of the scope may be stored in a database of a server, and either the model number or serial number may be used as a lookup key to retrieve this configuration data and collection of parameters. In other cases, the operating parameters may be separately individualized to each individual scope. For example, at the beginning of an arthroscopic surgery on a shoulder, the IPU may confirm that the scope to be used is indeed an arthroscope of suitable diameter, length, and optical capabilities. The two approaches may be combined, so that some parameters are stored based on model number, and others are stored individually per scope.

Data stored in on-board memory or in a remotely-accessible database may include:
A unique serial number or database lookup key
A model number and version number (integer or ASCII)
A text description of the component’ Model Number/Model identifier that can be displayed on the control display screen (typically a 32-character ASCII string)
Calibration/normalization data
Full configuration specifications—for example:
   The manufacturer's part number for the image sensor, which may allow many additional properties of the image sensor to be looked up in a table in the IPU including:
   The size (in rows×columns) of the image sensor
   Supported frame rates for the sensor and frame reporting rates
   Minimum/maximum integration time and integration time configuration resolution (for example, 0.1 to 100 ms in increments of 1 ms)
   An identifier for illumination sources on board the scope—white, infrared, ultraviolet, individual colors, image plane resolution (720×480, 1280×720, etc.)
   An identifier for what sensors are in the image plane—for example, one bit on/off for each of red, green, blue, ICG infrared, and other colors as extended in future software updates
   Information to establish white balance, color correction gamma curves, coefficients for distortion correction, sensor color sensitivity, illumination color, etc.
   (Boolean) Does/does not provide an illumination source in the handpiece
   (Boolean) Does/does not provide a de-fogging heater in the handpiece
   (Boolean) Does/does not provide a rotation sensor in the handpiece
   (Boolean) Does/does not support focus control in the handpiece
Calibration/normalization data—for example
   Corrective data for variations in lens focus
   Correction coefficients to compensate for image sensor color sensitivity, illumination color, white balance, distortion correction
   LED illumination brightness coefficients
An identifier to enable/disable certain image enhancement parameters based on the hardware image configuration—this may be used to pre-configure image processing settings based on anticipated imaging application for this scope. For example, a bit vector may enable or disable optimization of resolution, contrast, smoothing, and other optical properties
Various size and length properties, which may be important to control water pressure and the like
Manufacturing date
Date and time of first use
Date and time of initial connection and disconnections
Length of scope use during the procedure

Storing the data in an on-board memory (rather than in an off-board database) may improve field-adaptability. On-board data storage may reduce the need for software updates to the IPU, and may improve robustness if scopes are used in parts of a hospital or facility that do not have reliable internet access. In some cases, especially where the endoscopes use interchangeable replaceable components (such as the interchangeable insertion shafts shown in FIGS. 2D and 2E), each individual replaceable component may have a machine-readable serial number.

Data stored in the handset may be encrypted, with a decryption key stored in the IPU. Encryption may improve safety and security, by preventing a malicious actor from corrupting the memory contents or otherwise interfering with proper operation of the system.

Data may be communicated either in a fixed-field binary protocol or in a “keyword=” protocol (analogous to JSON protocols for web pages).

The connector may be a standard connector (e.g. USB-A) or a special purpose connector. A special purpose connector may ensure that mismatched devices are not plugged together. A special-purpose connector may allow additional pins to support all required signals and video, for example, video signals over twisted pair, higher current to power to a heater in the handset, and an optical connector for illumination light fibers.

### XIII.B. Use of Machine-Readable Serial Number to Reduce Errors, Ensure Non-Expiration and Ensure Sterile Single-Use

The machine-readable serial number may be used to improve efficiency of initiating a new procedure. When the scope is about to be used, the serial number may be scanned, either as a 2D optical bar code on the box, enclosed in packaging, or the scope itself, or via a remote sensing (for example, an RFID tag), or it may be read from EEPROM or similar nonvolatile memory onboard the scope itself, for example, as the scope is plugged into the IPU. As an alternative, the box or packaging may have printed information such as product model number, lot, manufacture date, expiration date and serial number, that allows redundancy in case the machine-readable information cannot be read.

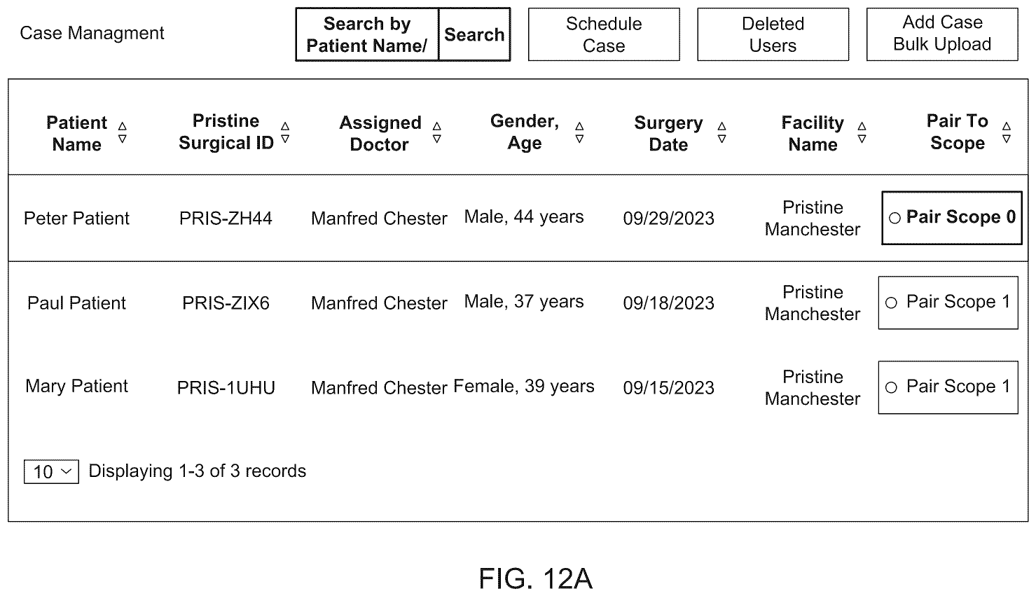

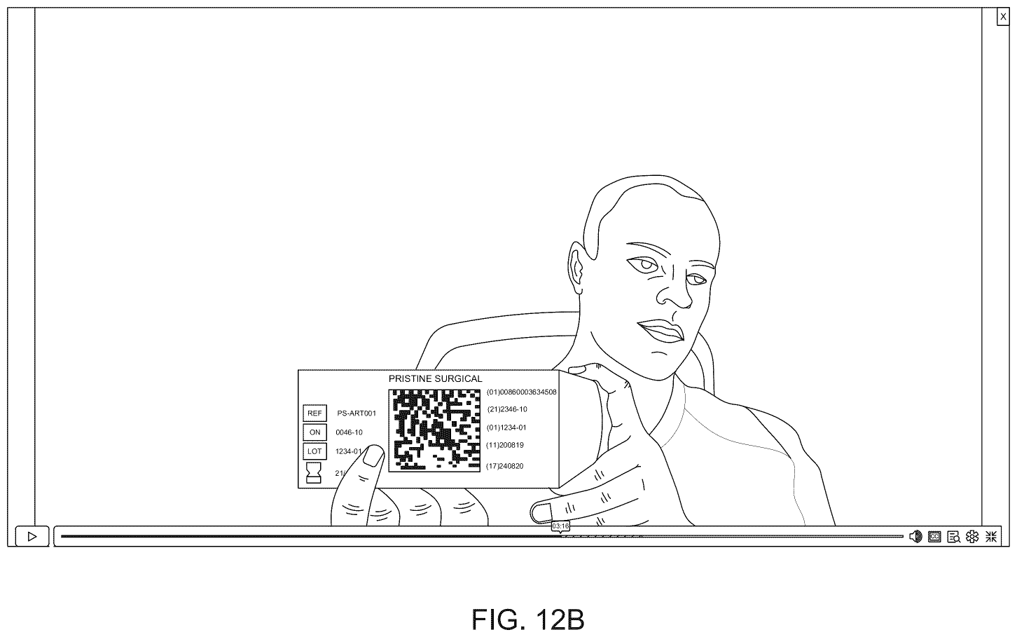

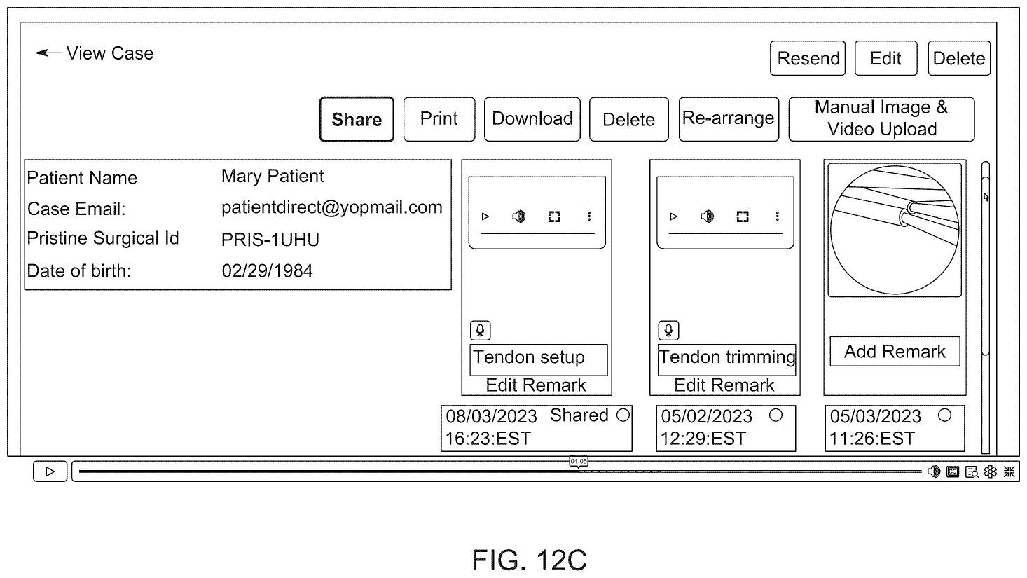

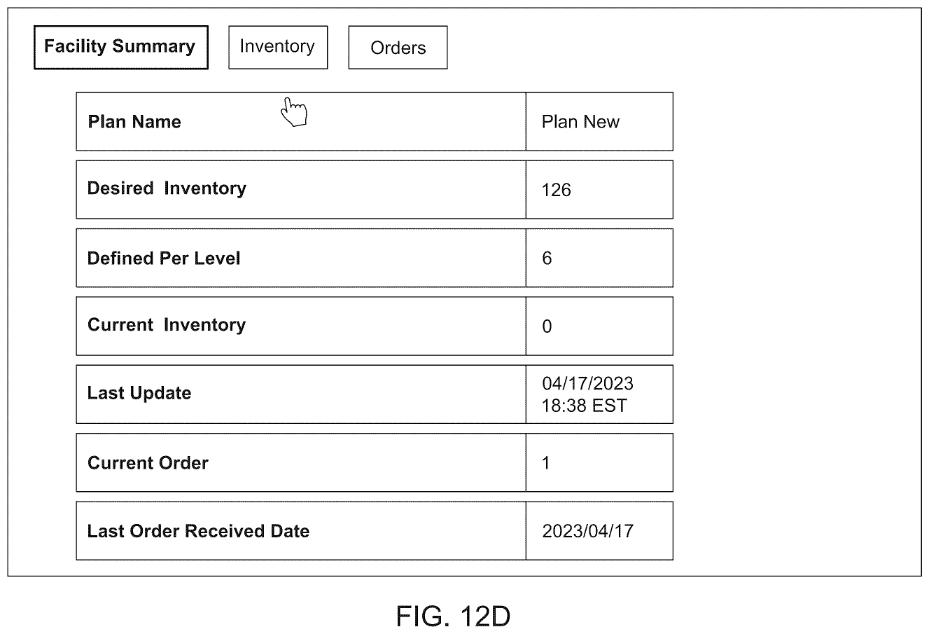

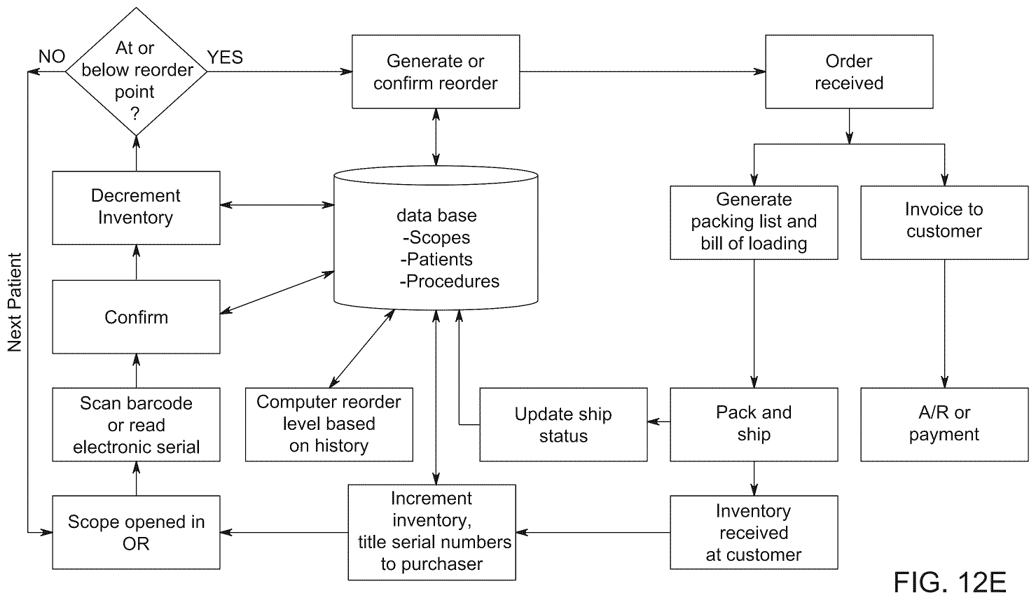

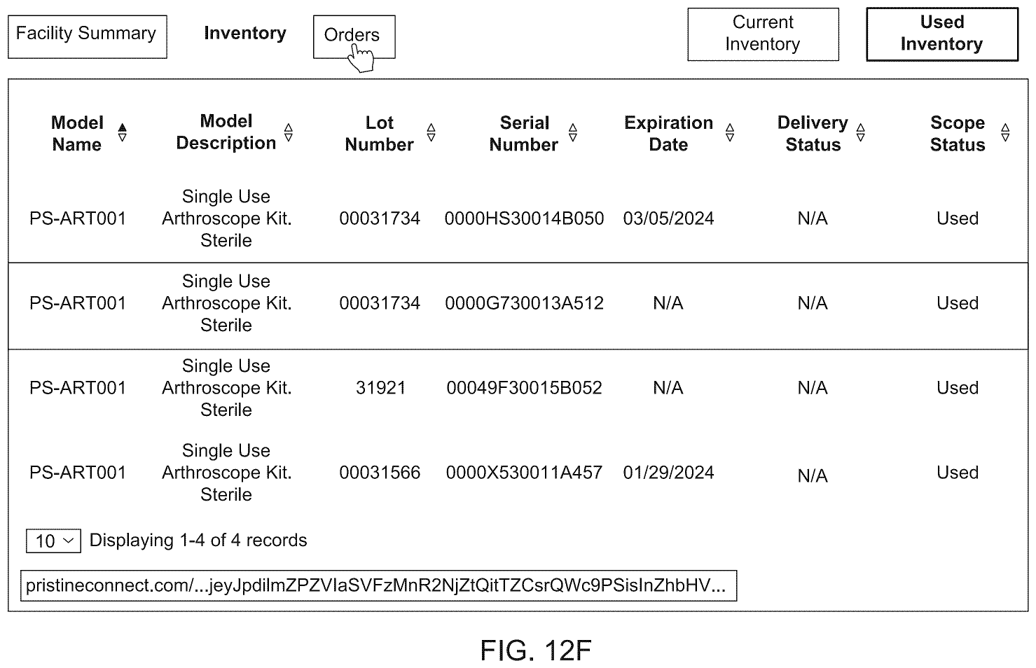

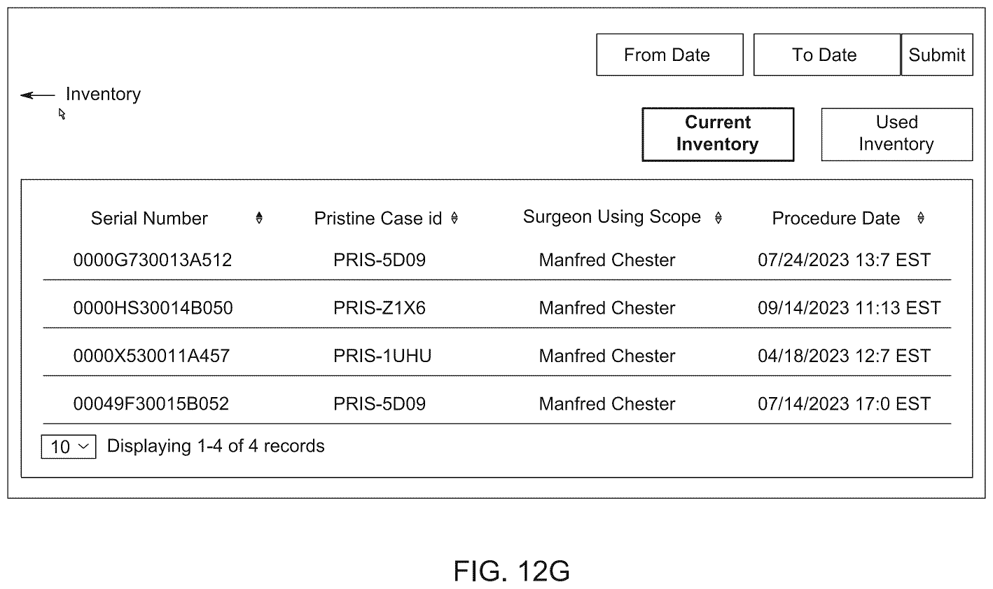

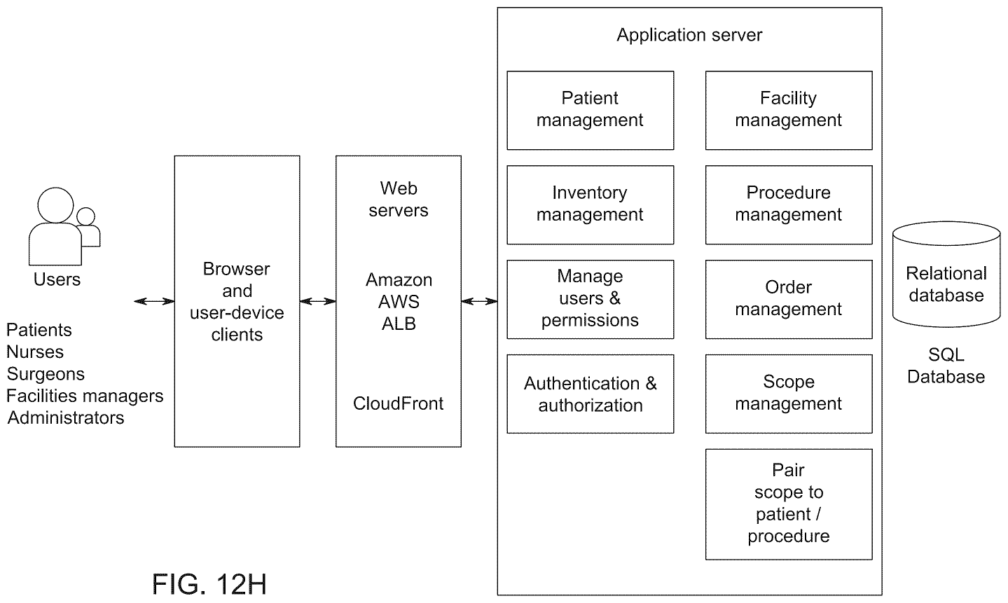

Referring to FIGS. 12A and 12B, as one example of pairing a scope with a specific patient and procedure, a member of surgical staff may request a computer display of a set of patient names, for example, the patients scheduled to be seen during the next few hours. The staff member may click on a button associated with the specific patient to be paired to the scope. The computer may respond by turning on a camera and asking the staff member to present a Matrix (2D) barcode on scope packaging to the camera. When the camera finds the Matrix (2D) barcode, the computer may pair the scope associated with that Matrix (2D) barcode with that patient and the patient's medical record. Once the human patient, patient electronic medical record, and scope are paired to each other, one or more of the following operations may occur:
Database records for the patient and scope may be linked to each other.
The database record for the scope may be marked to indicate that the scope is about to be used.
The database may be queried to ensure that the scope has not been previously paired to a different patient. (In some cases, software may provide an emergency override so long as the scope has not been placed in actual use with another patient.)
The computer may make a number of validation checks and use constraints:
   Based on the machine-readable serial number and the database, a computer may check whether this scope is assigned to the facility/location at which use has been initiated.
   Based on the machine-readable serial number and the database, a computer may check whether the scope to be used is appropriate for the procedure for which the patient is scheduled. For example, if the scope is an arthroscope for joints and the surgeon is a GI physician or the scheduled procedure is a colonoscopy, the computer may flag a warning to the human user and seek confirmation.
   The computer may check that all expiration dates are satisfied, that the scope is not subject to a recall or other product actions.
The database record for the scope may be updated to indicate a storage location for video, still images, or voice dictation to be stored in association with the patient's medical record.

In another example, the computer may read the 2D barcode or other machine-readable optical code first, and based on that code, present a list of patients that are scheduled for procedures using that form of scope, or may request a patient identifier such as name, patient record number, or the like.

When the scope is plugged into the IPU for use, the steps and cross-checks listed above may be performed or repeated. If the scope was not previously paired to the patient, then the checks may be performed for the first time, and the computer may prompt a human user to provide the patient identity. In addition, in the database record for the scope, initial connection to the IPU may cause the following updates to a database of scope data:
A flag may be set to indicate that the scope has been removed from its packaging and is no longer sterile.
If the scope is sold for single use, to ensure sterility, reliability, the fact that use has been initiated may be recorded at the manufacturer's database and/or in the memory of the scope itself. That single use may be recorded as a single binary flag, that, when set, forbids further use.
The first use may be marked as a timestamp and/or location, so that in some period of time (for example two or four hours), the scope cannot be reused. This would allow for the scope to be plugged in multiple times during a single procedure (for example to untangle a cable, or to reset after a power failure), but still be sufficient to prevent reuse.

As a procedure begins, or when a scope is plugged into the IPU, the IPU may run through a dialog to confirm that the scope and procedure are appropriate for each other. For example, the IPU may query the patient's electronic medical record to confirm the procedure to be performed, and confirm that the attached scope is appropriate for the procedure. If a mismatch is detected, the IPU may offer a warning and request a confirmation and override. The serial number of the exact scope used may be stored in the medical record in case of an audit issue.

During a procedure, the scope or IPU may encounter an error or failure. In such event, the IPU or scope may communicate with a computer or database to initiate complaint handling or feedback process with manufacturer, hospital or appropriate reporting body. Alternatively, software may provide an ability for a user to initiate a feedback event. An error code may be stored in the medical record in case of audit.

The stored data may allow a single IPU to be useable with multiple scope configurations, reducing complexity of stocking, supplying, and using different scopes for different purposes.

If the scope is refurbished, that flag can be cleared to allow reuse.

The machine-readable serial number may provide an index to the manufacturer's literature library. This can provide near instant access to information should it be required during a surgical procedure.

XIII.C. Use of Machine-Readable Serial Number to Communicate Patient Data into Electronic Medical Record

Referring to FIG. 12C, the pairing of the scope to the patient may improve efficiency of getting information into the patient's electronic medical record. During the procedure, the surgeon or an assistant may mark the entirety or marked portions of the video for permanent storage in the patient's electronic medical record, or into another database maintained by the hospital/customer or the scope manufacturer. In some cases, the IPU may compute voice-to-text of the physician's narration during the procedure. The IPU may connect to a cloud application via Wi-Fi or Ethernet. Images and videos may be sent to this cloud application in real time, after each procedure, or stored on the USB memory. The images and video may be sent to the cloud application as a live stream, or may be collected in storage in the IPU for periodic uploading, such as at the end of the day. Images and video may be stored with the patient's electronic medical record, either in raw form, or after some selection and editing. They may be annotated with text or dictation by a physician or assistant. The video or images from the procedure may be automatically routed to storage associated with a specific patient (such as the patient's electronic medical record, or a patient portal hosted via a database separate from the hospital's or surgeon's electronic medical record) based on the pairing established by means of the machine-readable serial number.

This video may be anonymized, edited and/or delivered to the patient, perhaps with voice over dictation, as described in patent application Ser. No. 16/278,112, filed Feb. 17, 2019, incorporated by reference. This video may improve the patient's post-operative rehab, and may provide patient-specific reporting.

### XIII.D. Use of Machine-Readable Serial Number for Inventory Control, Location Tracking, Reordering, and Stock Management

Referring to FIGS. 12D and 12E, the purchaser/hospital may interact with the database to set a minimum inventory level. Alternatively, a computer system accessible to the manufacturer may ascertain an average rate of use, time for delivery based on location, and any pending or in-transit inventory, to compute a reorder inventory level. As each scope is used, one or more computers may decrement the existing stock level, and if that decremented level, compared against the reorder stock level, suggests reorder, the computer may automatically enter a reorder to maintain the stock at a proper level. The computer may integrate via an API or similar EDI (electronic data interchange) connection with a distribution center or 3^{rd} party to place reordered quantity. A given user ID may have one or more billing locations and one or more delivery addresses.

Scope packages may be scanned as necessary, typically as scopes arrive at the hospital/purchaser site, so that inventory can be checked in, and as inventory is moved from one internal location to another (for example, store rooms on different floors or wings). Additionally, the system may use tracking information from UPS or FedEx or another shipper/logistics manager to determine location of in-transit inventory from manufacturer, though the distribution chain to the final hospital/purchaser. The system may use tracking proof of delivery as a signal that a product was received by the customer site.

The system may issue a warning if it detects that a scope seems to have gotten lost. For example, the system may compute a typical inventory time for a given location (for example, perhaps two weeks), and may notice if one scope has not been scanned or moved for some multiple of that time. Similarly, the system may warn for unexpected inventory movement. The system may be programmed to eliminate false positives and over-reporting—for example, movement to a shipping hub, or movement via a hospital's internal distribution system may take a scope on an unexpected route, but should be suppressed to avoid over-reporting.

Referring to FIGS. 12F and 12G, the system may provide dashboards to provide real-time data to surgeons, facility managers and staff, and the supply chain, to provide ready information about inventory levels and flow through the supply chain. For example, dashboards may give orders placed and not yet received, shipment tracking information, delivery estimates, current on-hand inventor levels, and recent use patterns. FIG. 12F shows an inventory list of current inventory of scopes on hand ready for use, including model name, model description, lot number (typically useful in case of a product recall), serial number, and expiration date. FIG. 12G shows recently used scopes—the serial number, and where, when, and by whom used. This information may be presented at a total or statistical level, or as a list of individual scopes and their current status.

This tracking may improve utilization and inventory management by ensuring “just in time” ordering.

A database may store information that tracks the history of the scope. If the serial number is remotely scannable (for example, in an RFID tag), then the location of the scope may be tracked through the distribution channel and storage at the purchaser hospital. This information may be used to ensure that the scope has not exceeded any time limits, that it has not been stored in locations that were known to go over temperature limits, etc. For example, the 24-hour limit after first use may be enforced by the IPU by reading the time of first use from the non-volatile memory on the handle board PCBA. As a procedure begins, the IPU may do a query over the internet to confirm the scope has not exceeded a manufacturer's date, and that the scope remains within specification and is not subject to any safety recall.

### XIII.E. Configuration/Capability Information

Each machine-readable serial number may be related to information describing specific configuration and/or capability information relating to the scope. The serial number may be used to trace the device back to the records of how it was built and the components used in the build. In some cases, a reusable scope handle may be designed to be used with interchangeable parts, and each interchangeable or replacement component may have a machine-readable serial number, which, in turn, may be related to a database record with a specific model number, and that model number's database record may have a list of specific configuration and/or capability information. This information can be checked as the scope is paired to a patient or a similar time when specific capabilities of a specific scope or component should be compared to the specific needs of a patient. The serial number may be used to associate with a patient and to track inventory.

XIII.F. Calibration information

During manufacture, specific measurements and calibration data may be measured. For example, white balance, general level setting, color correction gamma curves, coefficients for distortion correction, sensor color sensitivity, illumination color, any anomalies of focus in different parts of the visual field, and the like. These data may be stored in a database indexed by the scope's serial number, so that it may be available to the image processing pipeline for compensation.

### XIII.G. Implementation

A database for implementation of the above features may include tables that track information such as the following:

| Table Name | Data Included |
|---|---|
| scope_product_id | Properties of a class of scopes: model name and |
| | number, list price, video resolution, video |
| | color scheme, surgical indications for which the |
| | scope is appropriate, etc. |
| scope_device | the structure that tracks the information for an |
| | individual scope: serial number, date of |
| | manufacture, date of sale, expiration date, actual |
| | sell price, , and relates an individual scope to |
| | orders, the facility thatnowns the scope, the |
| | product ID, manufacture date, |
| procedure_type | Information about a class of procedure: billing code, |
| | which scope_product_ids are suitable for the |
| | procedure type, which physicians specialize in that |
| | procedure, etc. |
| procedure | the table that creates the one-to-many relation |
| | between an individual patient, the procedures that |
| | have been performed on that patient, the scopes |
| | used in each procedure, data related to each |
| | specific procedures and scope uses, and the data |
| | created as a result of usage of a scope for a patient |
| scope_order_{—} | The table that tracks sales orders from booking |
| management | of the sale through manufacture and shipping to |
| | delivery and storage in the facility's inventory |
| patient | Information about a specific patient, with one- |
| | to-many links to procedures, scopes, and the like |
| facility_scope_{—} | for each scope_product_id and facility, the |
| inventory | stock levels |

The database may have tables describing scopes. One table may describe properties of models of scope, properties that are common to a product ID class of scopes. A second table may describe individual scopes.

Table scope_product_id

| Field | Type | Data Included |
|---|---|---|
| scope_id | | |
| scope_mode1 | text | The scope's model number |
| scope_name | text | The scope's model name |
| scope_udi | integer | universal product ID |
| list_price | integer | undiscounted list price |
| gtin | integer | Global Trade Item Number |
| video_color_{—} | integer | is the camera of this scope class RGB |
| scheme | | or CYM? |
| video_resolution | integer | code to identify 720×480, 1280×720, |
| | | etc |
| surgical_{—} | relation | one-to-many link to surgical |
| indications | | indications for which this class |
| | | is appropriate, used as an error check |
| scope_device | relation | a one-to-many link to all “scope_device” |
| | | records, for all scopes of this |
| | | scope_product_id |
| regulatory | relation | link to a row of table of regulatory |
| | | issues: experimental vs approved, |
| | | export restrictions, etc |

Table scope_device

| Field | Type | Data Included |
|---|---|---|
| serial_number | text | Serial number for the scope |
| product_id | relation | link to a row of table “scope_product_id” |
| | | for the class of this scope |
| order_id | relation | link to row of table “orders” |
| lot_number | | Manufacturing lot number (e.g. for recall) |
| facility_id | relation | link to row of table “facility” to |
| | | indicate the owner of this scope |
| order | relation | link to row of table “scope_order_{—} |
| | | management” to show the order in which |
| | | this scope is shipped |
| scope_price | | actual sell price |
| date_manufacture | date | date of manufacture |
| scope_white_{—} | | RGB or CYM or similar numerical |
| balance | | representation of the white balance for |
| | | compensation by the image pipeline |
| other | | other parameters/calibration values |
| | | measured during manufacture such as |
| | | color correction gamma curves, |
| | | coefficients for distortion correction, |
| | | sensor color sensitivity, illumination color |
| expiry_date | date | date of expiration |
| status | integer | integer code indicating status |

The database may have tables describing procedures. One table may describe properties of a class of procedures. A second table may describe individual procedures on individual patients.

Table procedure_type

| Field | Type | Data Included |
|---|---|---|
| procedure_name | text | name of procedure |
| billing_code | integer | AAPC/CPT/HCPS category code |
| [other] | | links to table “scope_product_id” for |
| | | scopes useable for this procedure, surgeons |
| | | and facilities authorized to perform this |
| | | procedure, etc. |
| status | integer | status |
| is_deleted | boolean | |

Table procedure

| Field | Type | Data Included |
|---|---|---|
| procedure_type | relation | link to row of table “procedure_type” |
| procedure_name | text | Name of procedure (if different than the |
| | | name from “procedure_type”) |
| patient | relation | link to a row of table “patient” for the |
| | | patient of this procedure |
| scope | relation | link to a row of table “scope_device” for |
| | | the specific scope used in this procedure |
| facility_id | relation | link to a row of table “facility” for the |
| | | facility at which the procedure is performed |
| procedure_date | date | date on which the procedure performed |
| status | integer | status |
| surgeon_id | | link to a row of table “surgeon” |
| is_deleted | boolean | |

The database may have tables describing orders and inventory states.

Table scope_order_management

| Field | Type | Data Included |
|---|---|---|
| scopes | relation | link to one-to-many relation to the |
| | | scopes shipped in this order |
| facility_id | relation | link to a row of table “facility” |
| | | indicating the facility that owns the |
| | | scope |
| customer_po | relation | link to image of purchase order |
| customer_po_id | text | the customer's purchase order |
| | | number |
| date_po | date | the purchase order date |
| date_sold | date | date of sale |
| date_po_received | date | date received by purchaser |
| bill_to_id | relation | link to purchaser |
| bill_of_lading | relation | link to image of bill of lading |
| ship_to_id | relation | link to row of table of addressees |
| date_shipped | date | date order shipped |
| carrier_requested | relation | link to a row of table of carriers |
| requested_receive_date | date | customer's requested date for receipt |
| price | | total price |
| no_of_scope | integer | total number of scopes |
| shipment_weight | num | shipping weight |
| status | integer | a status |
| shipper_tracking_num | text | the shipper's tracking number |
| trackmore_api_status | | link to API for tracking shipment |
| delivered_status | integer | status of shipment and delivery |
| date_delivered | date | date delivered |
| expiry_date | date | date to expire this database |

Table facility_scope_inventory

| Field | Type | Data Included |
|---|---|---|
| scope_product_id | relation | link to a row of table “scope_product_id” |
| | | for the class of this scope |
| facility_id | relation | link to a row of table “facility |
| inventory | integer | |
| current_inventory | integer | current number of “product_id” scopes at |
| | | facility “facility_id” |
| par_level | integer | Par stock level |
| reorder_level | integer | Reorder when inventory reaches this level |
| status | integer | a status |

The database may have tables describing patients.

Table patient

| Field | Type | Data Included |
|---|---|---|
| patient_id | integer | A serial or record number |
| patient_name | text | Patient name |
| patient_dob | date | Patient date of birth |
| patient_gender | text | Patient gender |
| patient_email | text | Patient email |
| patient_phone | integer | Patient phone number |
| password | relation | link to an encrypted table |
| symptom | relation | link to a row in table “symptom” |
| procedure | relation | link to a one-to-many relation to the |
| | | patient's procedures |

The database may have tables that set up the relations between scopes, procedures, patients, surgeons, inventory states, and the like.

Other tables may represent surgeons, surgical specialties, surgical offices, surgical staff members, system administrators, addresses, passwords and similar security information; links to link video, voice dictation, and images into patient medical records; procedure checklists, access permissions so that patients are able to see only the patient information to which each is entitled, and the like.

Referring to FIG. 12H, an architecture of a system may be arranged around client-server principles. Users may interact with user-device client software, such as browser HTML or a downloaded app on either a phone or a desktop computer. On the server side, the front-end may be provided by off-the-shelf components such as Amazon AWS, ALB (application load balancer), and/or CloudFront content delivery network. The application server may include components for patient management, inventory management, management of user IDs, permissions, authentication, and authorization; facility management; procedure management; order management; scope management; pairing between scopes and patients and procedures, and other administrative functions. A relational/SQL database may provide storage and lookup.

### XIV. Embodiments

Embodiments of the invention may include any one or more of the following features, singly or in any combination.

Endoscope 100 may have a handle, and an insertion shaft, the insertion shaft having at its distal end a camera. The insertion shaft may have solid state illumination and imaging circuitry at or near a tip designed to provide illumination and imaging of the interior of a body cavity for a surgeon during surgery. The proximal portion of the handle may have electronics for drive of the illumination circuitry and to receive imaging signal from the imaging circuitry. The proximal handle portion may be designed to permit sterilization between uses. A joint between the proximal handle portion and the insertion shaft may designed to separably connect the insertion shaft to the proximal handle portion. When it is separated, the joint may permit removal of the insertion shaft for disposal and replacement. The joint may be designed so that, when connected, the joint can transfer mechanical force from a surgeon's hand to the insertion shaft, and provides electrical connectivity between the proximal handle circuitry and the illumination and imaging circuitry. The handle may have proximal and distal portions. The distal portion may lie between the insertion shaft and proximal handle portion. The insertion shaft may be rigidly affixed to the distal handle portion. The joint may be disposed to connect and disconnect the distal and proximal portions of the handle. The distal handle portion may be designed to indirectly transfer mechanical force between a surgeon's hand to the insertion shaft, and provide indirect electrical connectivity between the proximal handle circuitry and the illumination and imaging circuitry. The handle may have a rotation collar having surface features designed to assist the surgeon in rotating the insertion shaft in the roll dimension about the axis of the insertion shaft relative to the proximal handle portion. The electronics inside the proximal handle portion may be designed to sense roll of the insertion shaft, and provide an angular rotation signal designed to permit righting of a displayed image received from the imaging circuitry. A mounting for the image sensor may be designed to permit panning of the image sensor about a pitch or yaw axis perpendicular to the central axis of the insertion shaft. One or more ultraviolet LEDs internal to the endoscope may be designed to sterilize a region of the interior of the endoscope. Hoses for insufflation fluid or gas may be designed on lie on or near a central axis of proximal handle portion. Two or more insertion shafts each having dimensions different than the others, may each be connectable to the proximal handle portion at the joint, to permit use of the proximal handle in surgery with different requirements for insertion shaft. A sterilization cabinet may be designed to sterilize components of the endoscope. An insertion shaft of an endoscope tip has a rigid proximal portion and a distal portion. The distal portion is bendable to direct a field of view of imaging circuitry in a desired direction. An illuminator and solid state imaging circuitry are at or near a distal tip of the articulable distal portion. The illuminator is designed to illuminate, and the imaging circuitry being designed to capture imaging of, an interior of a body cavity for a surgeon during surgery. A coupling of the replaceable endoscope tip is designed to separably connect the insertion shaft at a joint to a handle portion, and to disconnect the joint. The coupling has mechanical connectors. When the joint is separated, the mechanical connectors permit removal of the insertion shaft from the handle for disposal and replacement. When the joint is connected, the joint is designed to provide mechanical force transfer between a surgeon's hand to the insertion shaft. Electrical connectors are designed to connect the insertion shaft to electronics in the handle. The handle electronics are designed for drive of the illuminator and to receive imaging signal from the imaging circuitry, the handle being designed to permit sterilization between uses. Control force transfer elements are designed to permit a surgeon to direct a direction of the imaging circuitry by transfer of mechanical force directed by a surgeon to the articulable distal portion. The distal bendable portion includes a series of articulated rigid segments. A sheath or cover over the articulated rigid segments is designed to reduce intrusion or pinching. The distal bendable portion is formed of a solid component, bendable in its lateral and elevation dimensions, and relatively incompressible in compression in its longitudinal dimension. The distal bendable portion is extendable from and retractable into a solid sheath. The distal bendable portion is bendable in one dimension. The distal bendable portion is bendable in two orthogonal dimensions. The imaging circuitry is mounted within at or near a distal tip of the articulable distal portion via a pannable mounting. The pannable mounting is designed as two sides of a parallelogram. The imaging circuitry is mounted on a structural segment hinged to the two parallelogram sides. Passages and apertures are designed to pass irrigation fluid to improve view from a lens or window over the imaging circuitry. Passages and apertures are designed to pass inflation fluid to enlarge a cavity for surgery. Mechanical connectors of the coupling include a twist-lock designed to affix the endoscope insertion shaft to the handle portion. A plurality of the endoscope tips are bundled and packaged together with a handle. The handle has electronics designed for drive of the illuminator and to receive imaging signal from the imaging circuitry. The plurality of tips and handle are packaged for integrated shipment and sale. The illuminator is an illumination LED mounted at or near the distal tip. The illuminator is an emission end of a fiber optic fiber driven by an illumination source in the handle. Camera 410 may be enclosed within a plastic casing. The plastic casing may be formed as an overmolded jacket that is designed to protect camera 410 from bodily fluids and to structurally hold components of the tip in an operating configuration. The overmolded jacket may be designed to retain a transparent window in operating configuration with camera 410. The overmolded component may be formed of transparent plastic. The overmolded component may be designed to function as a lens for image sensor 410. Image sensor 410 may be mounted on a flexible circuit board. Flexible circuit board 416 may mount an illumination LED 418. LED 418 and image sensor 410 may be mounted on opposite sides of flexible circuit board 416. Image sensor 410 may be protected behind a transparent window. The window may be molded in two thicknesses, a thinner portion designed for mounting and to allow passage of illumination light, a thicker portion over camera 410. The handle may contain a circuit board with circuitry for control of and receipt of signals from camera 410. The handle and its components may be designed with no metal fasteners, and no adhesives, except those captured by overmolding. Control buttons of the endoscope may be molded with projections that function as return springs. The projections may be adhered into the endoscope handle via melting. The circuit board may be overmolded by plastic that encapsulate the circuit board from contact with water. The circuit board may be mounted into the handle via melting. Components of the handle may be joined to each other into a unitary structure via melting. Components of the handle may be joined by resilient clips designed to held the two components to each other before joining into unitary structure via melting. The handle may be formed of two shells concentric with each other. Rotation of the two shells relative to each other may be controlled via one or more O-rings frictionally engaged with the two respective shells. The handle may have overmolded a layer of a high-friction elastomer. The insertion shaft may be connected to the handle via a separable joint. A water joint of the separable joint may be molded for an interference seal without O-rings. A water cavity of the separable joint may be designed to impart swirl to water flowing from the handle to the insertion shaft. The insertion shaft may be formed of stainless steel and connected to the handle via a separable joint. Plastic components of the endoscope may be joined to the insertion shaft via overmolding of plastic into slots aligned at an oblique angle in the wall of the insertion shaft, without adhesives. The water joint may be formed as two cones in interference fit. The cones may interfere at a large diameter. The cones may interfere via a ridge raised on a lip of the inner male cone. Obturator 104 may be designed to pierce tissue for introduction of the endoscope. Features for twist-locking obturator 104 into trocar 102 may be compatible with features for twist-locking the endoscope into trocar.

An endoscope may have a handle and an insertion shaft. The insertion shaft has solid state illumination and imaging circuitry at or near a tip designed to provide illumination and imaging of the interior of a body cavity for a surgeon during surgery. The proximal portion of the handle has electronics for drive of the illumination circuitry and to receive a video signal from the image sensor, the proximal handle portion being designed to permit sterilization between uses. A joint between the proximal handle portion and the insertion shaft is designed to separably connect the insertion shaft to the proximal handle portion. When it is separated, the joint permits removal of the insertion shaft for disposal and replacement. The joint is designed so that, when connected, the joint can transfer mechanical force from a surgeon's hand to the insertion shaft, and provides electrical connectivity between the proximal handle circuitry and the illumination and imaging circuitry.

An endoscope may have a handle and an insertion shaft, the insertion shaft having solid state illumination and imaging circuitry at or near a tip designed to provide illumination and imaging of the interior of a body cavity for a surgeon during surgery, and the proximal portion of the handle having electronics for drive of the illumination circuitry and to receive a video signal from the image sensor, the proximal handle portion being designed to permit sterilization between uses; and a joint between the proximal handle portion and the insertion shaft designed to separably connect the insertion shaft to the proximal handle portion. The joint is separated to permit removal of the insertion shaft for disposal and replacement. The joint is reconnected with a new insertion shaft, the connection designed to provide mechanical force transfer between a surgeon's hand to the insertion shaft, and electrical connectivity between the proximal handle circuitry and the illumination and imaging circuitry.

Embodiments of the invention may include one or more of the following features. The handle may have proximal and distal portions. The distal portion may lie between the insertion shaft and proximal handle portion. The insertion shaft may be rigidly affixed to the distal handle portion. The joint may be disposed to connect and disconnect the distal and proximal portions of the handle. The distal handle portion may be designed to indirectly transfer mechanical force between a surgeon's hand to the insertion shaft, and provide indirect electrical connectivity between the proximal handle circuitry and the illumination and imaging circuitry. The handle may have a rotation collar having surface features designed to assist the surgeon in rotating the insertion shaft in the roll dimension about the axis of the insertion shaft relative to the proximal handle portion. The electronics inside the proximal handle portion may be designed to sense roll of the insertion shaft, and provide an angular rotation signal designed to permit righting of a displayed image received from the image sensor. A mounting for the image sensor may be designed to permit panning of the image sensor about a pitch or yaw axis perpendicular to the central axis of the insertion shaft. One or more ultraviolet LEDs internal to the endoscope may be designed to sterilize a region of the interior of the endoscope. Hoses for insufflation fluid or gas may be designed on lie on or near a central axis of proximal handle portion. Two or more insertion shafts each having dimensions different than the others, may each be connectable to the proximal handle portion at the joint, to permit use of the proximal handle in surgery with different requirements for insertion shaft. A sterilization cabinet may be designed to sterilize components of the endoscope.

An endoscope may have a handle, and an insertion shaft. The insertion shaft may have at its distal end a camera. Camera 410 may be enclosed within a plastic casing with an overmolded jacket that is designed to protect camera 410 from bodily fluids and to structurally hold components of the tip in an operating configuration. Camera 410 may be protected behind a transparent window. The window may be molded in two thicknesses. A thinner portion designed for mounting and to allow passage of illumination light, a thicker portion over camera 410. The handle may have retained within a circuit board with circuitry for control of and receipt of signals from camera 410. The handle and its components may be designed with no metal fasteners, and no adhesives, except those captured by overmolding. The handle may be formed of two shells concentric with each other. Rotation of the two shells relative to each other may be controlled via one or more O-rings frictionally engaged with the two respective shells. The handle may have an overmolded layer of a high-friction elastomer. The insertion shaft may be connected to the handle via a separable joint, a water joint of the separable joint may be molded for an interference seal without O-rings. The insertion shaft may be connected to the handle via a separable joint. A water cavity of the separable joint may be designed to impart swirl to water flowing from the handle to the insertion shaft. The insertion shaft may be formed of stainless steel and connected to the handle via a separable joint. Plastic components of the endoscope may be joined to the insertion shaft via overmolding of plastic into slots aligned at an oblique angle in the wall of the insertion shaft, without adhesives. The insertion shaft may be connected to the handle via a separable joint. Obturator 104 may be designed to pierce tissue for introduction of the endoscope. Features for twist-locking obturator 104 into trocar 102 may be compatible with features for twist-locking the endoscope into trocar 102.

The overmolded jacket may be designed to retain a transparent window in operating configuration with camera 410. The overmolded component may be formed of transparent plastic and designed to function as a lens for camera 410. Camera 410 may be mounted on a flexible circuit board: Flexible circuit board 416 may have mounted thereon an illumination LED 418. LED and camera 410 may be mounted on opposite sides of flexible circuit board 416. Control buttons of the endoscope may be molded with projections that function as return springs, the projections to be adhered into the endoscope handle via melting. The circuit board may be overmolded by plastic that encapsulates the circuit board from contact with water. The circuit board may be mounted into the handle via melting. Components of the handle may be joined to each other into a unitary structure via melting Components of the handle may be further joined by resilient clips designed to held the two components to each other before joining into unitary structure via melting. The joint may be formed as two frusta of cones in interference fit. The two frusta may interfere at their large diameters. The frusta may interfering via a ridge raised on a lip of the inner male cone.

An endoscope may have a handle and an insertion shaft. The insertion shaft has solid state illumination and imaging circuitry at or near a tip designed to provide illumination and imaging of the interior of a body cavity for a surgeon during surgery. The proximal portion of the handle has electronics for drive of the illumination circuitry and to receive imaging signal from the imaging circuitry, the proximal handle portion may be designed to permit sterilization between uses. A joint between the proximal handle portion and the insertion shaft is designed to separably connect the insertion shaft to the proximal handle portion. When it is separated, the joint permits removal of the insertion shaft for disposal and replacement. The joint is designed so that, when connected, the joint can transfer mechanical force from a surgeon's hand to the insertion shaft, and provides electrical connectivity between the proximal handle circuitry and the illumination and imaging circuitry.

An endoscope may have a handle and an insertion shaft, the insertion shaft having solid state illumination and imaging circuitry at or near a tip designed to provide illumination and imaging of the interior of a body cavity for a surgeon during surgery. The proximal portion of the handle may have electronics for drive of the illumination circuitry and to receive imaging signal from the imaging circuitry. The proximal handle portion may be designed to permit sterilization between uses. A joint between the proximal handle portion and the insertion shaft designed to separably connect the insertion shaft to the proximal handle portion. The joint may be separated to permit removal of the insertion shaft for disposal and replacement. The joint may be reconnected with a new insertion shaft, the connection designed to provide mechanical force transfer between a surgeon's hand to the insertion shaft, and electrical connectivity between the proximal handle circuitry and the illumination and imaging circuitry.

Embodiments of the invention may include one or more of the following features. The handle may have proximal and distal portions. The distal portion may lie between the insertion shaft and proximal handle portion. The insertion shaft may be rigidly affixed to the distal handle portion. The joint may be disposed to connect and disconnect the distal and proximal portions of the handle. The distal handle portion may be designed to indirectly transfer mechanical force between a surgeon's hand to the insertion shaft, and provide indirect electrical connectivity between the proximal handle circuitry and the illumination and imaging circuitry. The handle may have a rotation collar having surface features designed to assist the surgeon in rotating the insertion shaft in the roll dimension about the axis of the insertion shaft relative to the proximal handle portion. The electronics inside the proximal handle portion may be designed to sense roll of the insertion shaft, and provide an angular rotation signal designed to permit righting of a displayed image received from the image sensor. A mounting for the image sensor may be designed to permit panning of the image sensor about a pitch or yaw axis perpendicular to the central axis of the insertion shaft. One or more ultraviolet LEDs internal to the endoscope may be designed to sterilize a region of the interior of the endoscope. Hoses for insufflation fluid or gas may be designed on lie on or near a central axis of proximal handle portion. Two or more insertion shafts each having dimensions different than the others, may each be connectable to the proximal handle portion at the joint, to permit use of the proximal handle in surgery with different requirements for insertion shaft. A sterilization cabinet may be designed to sterilize components of the endoscope.

A replaceable endoscope tip for an endoscope may have a rigid proximal portion and a distal portion. The distal portion may be bendable to direct a field of view of imaging circuitry in a desired direction. Illuminator and image sensor may be located at or near a distal tip of the articulable distal portion. The illuminator may be designed to illuminate, and the image sensor may be designed to capture imaging of, an interior of a body cavity for a surgeon during surgery. A coupling is designed to separably connect the replaceable endoscope tip at a joint to a handle portion, and to disconnect the joint. The coupling has mechanical connectors designed: (a) when separated, the mechanical connectors permitting removal of the replaceable endoscope tip from the handle for disposal and replacement; and (b) when connected, the joint designed to provide mechanical force transfer between a surgeon's hand to the insertion shaft. Electrical connectors are designed to connect the replaceable endoscope tip to electronics in the handle, the handle electronics designed for drive of the illuminator and to receive video signal from the image sensor, the handle may be designed to permit sterilization between uses. Control force transfer elements are designed to permit a surgeon to direct a direction of the imaging circuitry by transfer of mechanical force directed by a surgeon to the bendable distal portion.

An optical prism may be designed to displace a field of view offset angle of an endoscope. A connector is designed to affix the optical prism to a tip of an endoscope that has a field of view at an initial offset angle displaced off-axis of the endoscope, and to retain the optical prism against displacement forces during insertion of the endoscope into a body cavity. The optical prism and connector are designed to reduce the offset angle of the field of view of the endoscope toward on-axis relative to the initial offset when the prism and connector are affixed to an optical tip of the endoscope. The endoscope may be inserted into a body cavity. The endoscope has a field of view at an initial offset angle displaced off-axis of the endoscope. The endoscope has affixed to its distal end an optical prism designed to reduce the offset angle of the field of view of the endoscope toward on-axis relative to the initial offset. The prism is affixed to the distal end of the endoscope by a connector designed to retain the optical prism against displacement forces during insertion of the endoscope into a body cavity. The endoscope is withdrawn from the body with the prism affixed. The prism is removed from the endoscope. The endoscope is reinserted back into the body cavity with its field of view at the initial offset angle. The optical prism may be designed to reduce the offset angle of the endoscope's field of view to no more than 10°, or to no more than 5°, or to no more than 3°. The optical prism may be optically convex to magnify an image. The optical prism may be optically concave to enlarge the endoscope's field of view. The connector may be designed to affix to the endoscope by mechanical forces. An optical filter may be coupled with the prism. The endoscope may have a wetting surface designed to entrain an anti-adhesive lubricant in a layer over a lens or window of the endoscope. The wetting surface may be a porous solid. The porous solid may be formed by sintering or other heating of particles. The optical prism and connector may be affixed to the endoscope for shipment, and designed to retain an anti-adhesive lubricant in contact with a lens or window of the endoscope during shipment. The vial, well, or cavity may have a cap with a seal to seal around a shaft of the endoscope. The anti-adhesive lubricant may comprise silicone oil, or mixtures thereof. The anti-adhesive lubricant may comprise a mixture of silicone oils of different viscosities. The vial or cavity may include an optical prism designed to displace a field of view of an endoscope.

Packaging for an endoscope may have mechanical features designed to retain components of an endoscope, and to protect the endoscope for shipping and/or delivery. The packaging has a vial, well, or cavity designed to retain anti-adhesive lubricant in contact with a lens or window of the endoscope.

The distal bendable portion may include a series of articulated rigid segments. A sheath or cover may cover the articulated rigid segments designed to reduce intrusion or pinching. The distal bendable portion may be formed of a solid component, bendable in its lateral and elevation dimensions, and relatively incompressible in compression in its longitudinal dimension. The distal bendable portion may be extendable from and retractable into a solid sheath. The distal bendable portion may be bendable in one dimension. The distal bendable portion may be bendable in two orthogonal dimensions. The camera may be mounted within at or near a distal tip of the bendable distal portion via a pannable mounting. The pannable mounting may be designed as two sides of a parallelogram, and the camera may be mounted on a structural segment hinged to the two parallelogram sides. Passages and apertures may be designed to pass irrigation fluid to improve view from a lens or window over the imaging circuitry. Passages and apertures may be designed to pass inflation fluid to enlarge a cavity for surgery. Mechanical connectors of the coupling may include a twist-lock designed to affix the endoscope replaceable endoscope tip to the handle portion. A plurality of the endoscope replaceable endoscope tips may be packaged for integrated shipment and sale with a reusable handle, the handle having electronics designed for drive of the illuminator and to receive imaging signal from the imaging circuitry. The illuminator may be an illumination LED mounted at or near the distal tip. The illuminator may be an emission end of a fiber optic fiber driven by an illumination source in the handle.

An arthroscope may have a handle and an insertion shaft. The insertion shaft may have near its distal end a solid state camera. The shaft may enclosed therein light conductors designed to conduct illumination light to the distal end. The shaft may have an outer diameter of no more than 6 mm. The shaft may have rigidity and strength for insertion of the camera into joints for arthroscopic surgery. The light conductors in the region of the camera may be designed to conduct illumination light from a light fiber to the distal end through a space between the camera and the inner surface of the insertion shaft.

A light conduction fiber may have a flattened region shaped to lie between an endoscope camera and an inner surface of an outer wall of an endoscope shaft, and shaped to conduct illumination light to a distal end of the endoscope shaft for illumination of a surgical cavity to be viewed by the camera. The shaft may be no more than 6 mm in diameter. The flattened region is formed by heating a region of a plastic optical fiber, and squeezing the heated region in a polished mold.

Embodiments of the invention may include one or more of the following features. One or more light guides may be designed to conduct illumination light from a light fiber to the distal end. The light guide may have a cross-section other than circular. The light guide may have a coupling to accept illumination light from a circular-cross-section optical fiber. The light guide's cross-section in the region of the camera may be narrower than the diameter if the light fiber in the light guide's dimension corresponding to a radius of the insertion shaft. At least one of an inner and outer surface of the one or more light guides may be longitudinally fluted. A distal surface of the one or more light guides or flattened region may be designed to diffuse emitted light. A distal surface of the one or more light guides may have surface microdomes designed to diffuse emitted light, or may be otherwise configured to improve uniformity of illumination into a surgical cavity accessed by the arthroscope. One or more light conductors in the region of the camera may be formed as a flattened region of an optical fiber. The flattened region may be shaped to lie between the endoscope camera and an inner surface of an outer wall of an endoscope shaft. The flattened region may be shaped to conduct illumination light to a distal end of the endoscope shaft for illumination of a surgical cavity to be viewed by the camera. The shaft may be no more than 6 mm in outer diameter. The flattened region may be formed by heating a region of a plastic optical fiber. The flattened region may be formed by squeezing an optical fiber in a polished mold. Component parts for mounting near the distal end of the endoscope may be shaped using poka-yoke design principles to ensure correct assembly. Component parts of a lens assembly for mounting near the distal end may be shaped using poka-yoke design principles to ensure correct assembly. Component parts near the distal end may be formed to permit focus adjustment of a lens assembly during manufacturing. The endoscope may have a terminal window designed to seal with the shaft to prevent intrusion of bodily fluids, bodily tissues, and/or insufflation fluid. The terminal window may be designed to reduce optical artifacts. The artifacts may reduced may be reflection, light leakage within the endoscope, fouling by bodily fluids and/or bodily tissues, and fogging. The light conductors in the region of the camera may include at least one optical fiber of essentially continuous diameter from a light source, the light fibers being no more than about 0.5 mm diameter, and arrayed around or partially around the circumference of the distal end of the endoscope. An arthroscope insertion shaft may have near its distal end a camera. The shaft may have enclosed therein light conductors designed to conduct illumination light to the distal end. The shaft may have rigidity and strength for insertion of the camera into joints for arthroscopic surgery. The flattened region may be dimensioned to conduct illumination light from a light fiber to the distal end through a space between the camera and the inner surface of the insertion shaft.

An apparatus may include a computer processor and a memory. The processor is programmed to receive video image data from an image sensor at the distal end of an endoscope and to display the image data to a surgeon in real time. The processor is programmed to process the image data received from the image sensor via a machine learning model, the machine learning model trained to simultaneously upsample the image data to a resolution higher than that captured by the image sensor, to sharpen edges, and to enhance local contrast.

An apparatus may include a computer processor and a memory. The processor is programmed to receive video image data from an image sensor at the distal end of an endoscope and to display the image data to a surgeon in real time. The video image data have a frame rate at which the image data are generated by the image sensor. The processor is programmed to control the image sensor and/or an illumination source designed to illuminate a scene viewed by the image sensor, the controlling programmed to underexpose or overexpose every other frame of the video image data. The processor is programmed to process the image data received from the image sensor to combine successive pairs of frames of the image data to adjust dynamic range to enhance over-bright or over-dark portions of the image to expose detail, and to generate combined frames at the full frame rate of the video as generated by the image sensor.

An apparatus may include a computer processor and a memory. An apparatus may include a computer processor and a memory. The processor is programmed to receive video image data from an image sensor at the distal end of an endoscope and to display the image data to a surgeon in real time. The processor is programmed to sum an error for an intensity of the image relative to a setpoint intensity. The processor is programmed to simultaneously control at least two of gain, exposure, and illumination via a PID control algorithm to achieve image display at the setpoint intensity, maximum change per step of the PID control damped to prevent oscillation.

Embodiments may include one or more of the following features, singly or in any combination. The processor may be further programmed to control the image sensor and/or an illumination source designed to illuminate a scene viewed by the image sensor. The controlling may be programmed to underexpose or overexpose every other frame of the video image data. The processor may be further programmed to process the image data received from the image sensor to combine successive pairs of frames of the image data to adjust dynamic range to enhance over-bright or over-dark portions of the image to expose detail. The processor may be further programmed to generate combined frames at the full frame rate of the video as generated by the image sensor. The processor may be further programmed to sum an error for an intensity of the image relative to a setpoint intensity. The processor may be further programmed to simultaneously control at least two of gain, exposure, and illumination via a PID control algorithm to achieve image display at the setpoint intensity. A maximum change per step of the PID control may be damped to prevent oscillation. The processor may be further programmed to process the image data received from the image sensor via a machine learning model, the machine learning model trained to simultaneously upsample the image data to a resolution higher than that captured by the image sensor, to sharpen edges, and to enhance local contrast. The processor may be further programmed to enhance the video image data via dynamic range compensation. The processor may be further programmed to adjust exposure time, illumination intensity, and/or gain in image capture to adjust exposure saturation. The processor may be further programmed to enhance the video image data via noise reduction. The processor may be further programmed to enhance the video image data via lens correction. The processor may be further programmed to in addition to resolution, enhance at least two of dynamic range compensation, noise reduction, and lens correction. The processor may be further programmed to rotate the image display to compensate for rotation of the endoscope. The processor may be further programmed to adjust exposure time, illumination intensity, and/or gain in image capture to adjust exposure saturation.

A computer reads a machine-readable serial number of a medical device as the medical device is put into use. Based on the reading of the serial number, a computer records a reduction of an on-hand inventory level of a class of medical devices of which the medical device is a member. A computer computes when the reduction of on-hand inventory of the class indicates a level that warrants reordering to replenish the on-hand inventory. When warranted, a computer places an order with a supplier of medical devices of the class to replenish on-hand inventory of medical devices of the class.

A computer system one or more processors and a machine readable, non-transitory memory having stored therein instructions. The instructions are programmed to cause the processor(s) to read a machine-readable serial number of a medical device as the medical device is put into use, and based on the reading of the serial number, to record a reduction of an on-hand inventory level of a class of medical devices of which the medical device is a member. The instructions are programmed to cause the processor(s) to compute when the reduction of on-hand inventory of the class indicates a level that warrants reordering to replenish the on-hand inventory. The instructions are programmed to cause the processor(s) to place an order with a supplier of medical devices of the class to replenish on-hand inventory of medical devices of the class.

An endoscope has an image sensor designed to gather photons to produce video. The image sensor is of a type that has properties that vary from one sensor to another within manufacturing tolerances. The endoscope has a connector to connect the endoscope to a computer image processor. The image processor is programmed to obtain data from a database designed to store properties of the endoscope's image sensor and/or the image sensor's behavior, the database storing properties specific to specific sensors or a specific class of sensors. The obtained data describes the image sensor's properties from the database. The image processor is programmed to compute normalized video based on the obtained properties and the video from the image sensor.

A computer processor is programmed to receive image date from an image sensor of an endoscope, and to obtain data from a database designed to store properties of endoscopes' image sensors and/or the image sensors' behavior. The image sensor is of a type that has properties that vary from one sensor to another, the database storing properties specific to specific sensors or a specific class of sensors. A computer computes normalized video based on the obtained properties and the video from the image sensor.

A database stores information relating to specific individual medical devices, including physical location and/or ownership. An optical reader reads 2D identification codes on packaging of the medical devices. Database records are updated show current physical location and/or ownership based on the read 2D identification codes.

A computer system has one or more processors, and a machine readable, non-transitory memory having stored therein instructions. The instructions are programmed to cause the processor(s) to store information relating to specific individual medical devices, including physical location and/or ownership. An optical reader reads 2D identification codes on packaging of the medical devices. Database records are updated to show current physical location and/or ownership based on the read 2D identification codes.

A computer may read a machine-readable serial number of a endoscope as the endoscope is put into use. Based on the reading of the serial number, an on-hand inventory level of a class of endoscopes may be reduced and recorded. A reorder level may be computed based on the reduction in on-hand inventory level. A computer may compute when on-hand inventory level warrants reordering to replenish the on-hand inventory. Based on the computed warrant to reorder, a computer may place an order with a supplier of endoscopes of the class to replenish on-hand inventory of endoscopes of the class. The medical device may be an endoscope. The endoscope may have an image sensor. The image sensor may be designed to gather photons to produce video. The image sensor may be of a type that has properties that vary from one sensor to another within manufacturing tolerances. The programs may be programmed to connect a one of the medical devices or endoscopes to a computer processor. The processor may be programmed to obtain properties of the connected medical device from a database. The database may be designed to store information relating to specific individual medical devices, endoscopes, or image sensors. The database may be accessed based on the read serial number. The database may store properties of the endoscope's image sensor. The database may store properties of the image sensor's behavior in a database. The database may store properties specific to specific sensors. The database may provide properties of the one endoscope's image sensor from the database, and to compute normalized video based on the obtained properties and the video from the image sensor. The database may store physical location of individual medical devices, endoscopes, or components. The database may store physical location and/or ownership of individual medical devices, endoscopes, or components. An optical reader may read identification codes on packaging of the medical devices. Database records of physical location and/or ownership may be updated to show current physical location and/or ownership based on the read identification codes. When a medical device or endoscope may be connected to its control computer, the computer may be programmed to obtain properties of the connected device from a database. The identification code may be a machine-readable 2D optical code. The identification code may be stored in machine-readable electronic memory. The database stores information relating to a device model of a class of medical devices manufactured to be interchangeable. The database may be designed to index to the device model information based on the read identification code of an individual medical device. The database may be designed to store data describing a color spectrum capability of the image sensor. The database may be designed to store data describing an image plane resolution of the image sensor. The database may be designed to store calibration properties of the endoscope's image sensor specific to the specific image sensor of the specific endoscope. The image processor may be programmed to compute normalized video based on the obtained calibration properties and the video from the image sensor. The calibration properties may describe a white balance of the image sensor, or a color correction gamma curve of the image sensor, or a distortion correction of the image sensor, or any two or more, or any three or more of these properties. The computer may compute a prediction of delivery date based on the read 2D identification codes. The computer may update the physical location and/or ownership database records to show current physical location based on the read 2D identification codes or read of the machine-readable memory.

Various processes described herein may be implemented by appropriately programmed general purpose computers, special purpose computers, and computing devices. Typically a processor (e.g., one or more microprocessors, one or more microcontrollers, one or more digital signal processors) will receive instructions (e.g., from a memory or like device), and execute those instructions, thereby performing one or more processes defined by those instructions. Instructions may be embodied in one or more computer programs, one or more scripts, or in other forms. The processing may be performed on one or more microprocessors, central processing units (CPUs), computing devices, microcontrollers, digital signal processors, graphics processing units (GPUs), field programmable gate arrays (FPGAs), or like devices or any combination thereof. Programs that implement the processing, and the data operated on, may be stored and transmitted using a variety of media. In some cases, hard-wired circuitry or custom hardware may be used in place of, or in combination with, some or all of the software instructions that can implement the processes. Algorithms other than those described may be used.

Programs and data may be stored in various media appropriate to the purpose, or a combination of heterogeneous media that may be read and/or written by a computer, a processor or a like device. The media may include non-volatile media, volatile media, optical or magnetic media, dynamic random access memory (DRAM), static ram, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, other non-volatile memories, any other memory chip or cartridge or other memory technologies.

Databases may be implemented using database management systems or ad hoc memory organization schemes. Alternative database structures to those described may be readily employed. Databases may be stored locally or remotely from a device which accesses data in such a database.

In some cases, the processing may be performed in a network environment including a computer that is in communication (e.g., via a communications network) with one or more devices. The computer may communicate with the devices directly or indirectly, via any wired or wireless medium (e.g. the Internet, LAN, WAN or Ethernet, Token Ring, a telephone line, a cable line, a radio channel, an optical communications line, commercial on-line service providers, bulletin board systems, a satellite communications link, a combination of any of the above). Transmission media include coaxial cables, copper wire and fiber optics 430, including the wires that comprise a system bus coupled to the processor. Transmission may occur over transmission media, or over electromagnetic waves, such as via infrared, Wi-Fi, Bluetooth, and the like, at various frequencies using various protocols. Each of the devices may themselves comprise computers or other computing devices, such as those based on the Intel® Pentium® or Centrino™ processor, that are adapted to communicate with the computer. Any number and type of devices may be in communication with the computer.

A server computer or centralized authority may or may not be necessary or desirable. In various cases, the network may or may not include a central authority device. Various processing functions may be performed on a central authority server, one of several distributed servers, or other distributed devices.

The following applications are incorporated by reference. U.S. Provisional application Ser. No. 63/544,608, filed Oct. 17, 2023, titled Artificial Intelligence Agent for Preparing Physician's Note; U.S. application Ser. No. 18/370,375, filed Sep. 19, 2023, titled Image Enhancement for Endoscope; U.S. Provisional application Ser. No. 63/538,485, filed Sep. 14, 2023, titled Endoscope; U.S. Provisional application Ser. No. 63/534,855, filed Aug. 27, 2023, titled Endoscope; U.S. Provisional application Ser. No. 63/531,239, filed Aug. 7, 2023, titled Endoscope; U.S. Provisional application Ser. No. 63/437,115, filed Jan. 4, 2023, titled Endoscope with Identification and Configuration Information; U.S. application Ser. No. 17/954,893, filed Sep. 28, 2022, titled Illumination for Endoscope; U.S. Provisional App. Ser. No. 63/376,432, filed Sep. 20, 2022, titled Super Resolution for Endoscope Visualization; U.S. application Ser. No. 17/896,770, filed Aug. 26, 2022, titled Endoscope; U.S. Provisional App. Ser. No. 63/400,961, filed Aug. 25, 2022, titled Endoscope; U.S. application Ser. No. 17/824,857, filed May 25, 2022, titled Endoscope; U.S. Prov. App. Ser. No. 63/249,479, filed Sep. 28, 2021, titled Endoscope; U.S. Prov. App. Ser. No. 63/237,906, fled Aug. 27, 2021, titled Endoscope; U.S. application Ser. No. 17/361,711, filed Jun. 29, 2021, titled Endoscope with Bendable Camera Shaft; U.S. Prov. App. Ser. No. 63/214,296, filed Jun. 24, 2021, titled Endoscope with Bendable Camera Shaft; U.S. Provisional App. Ser. No. 63/193,387 titled Anti-adhesive Window or Lens for Endoscope Tip; U.S. Provisional App. Ser. No. 63/067,781, filed Aug. 19, 2020, titled Endoscope with Articulated Camera Shaft; U.S. Provisional Application Ser. No. 63/047,588, filed Jul. 2, 2020, titled Endoscope with Articulated Camera Shaft; U.S. Provisional App. Ser. No. 63/046,665, filed Jun. 30, 2020, titled Endoscope with Articulated Camera Shaft; U.S. application Ser. No. 16/434,766, filed Jun. 7, 2019, titled Endoscope with Disposable Camera Shaft and Reusable Handle; U.S. Provisional App. Ser. No. 62/850,326, filed May 20, 2019, titled Endoscope with Disposable Camera Shaft; U.S. application Ser. No. 16/069,220, filed Oct. 24, 2018, titled Anti-Fouling Endoscopes and Uses Thereof; U.S. Provisional App. Ser. No. 62/722,150, filed Aug. 23, 2018, titled Endoscope with Disposable Camera Shaft; U.S. Provisional App. Ser. No. 62/682,585 filed Jun. 8, 2018, titled Endoscope with Disposable Camera Shaft.

For clarity of explanation, the above description has focused on a representative sample of all possible embodiments, a sample that teaches the principles of the invention and conveys the best mode contemplated for carrying it out. The invention is not limited to the described embodiments. Well known features may not have been described in detail to avoid unnecessarily obscuring the principles relevant to the claimed invention. Throughout this application and its associated file history, when the term “invention” is used, it refers to the entire collection of ideas and principles described; in contrast, the formal definition of the exclusive protected property right is set forth in the claims, which exclusively control. The description has not attempted to exhaustively enumerate all possible variations. Other undescribed variations or modifications may be possible. Where multiple alternative embodiments are described, in many cases it will be possible to combine elements of different embodiments, or to combine elements of the embodiments described here with other modifications or variations that are not expressly described. A list of items does not imply that any or all of the items are mutually exclusive, nor that any or all of the items are comprehensive of any category, unless expressly specified otherwise. In many cases, one feature or group of features may be used separately from the entire apparatus or methods described. Many of those undescribed alternatives, variations, modifications, and equivalents are within the literal scope of the following claims, and others are equivalent. The claims may be practiced without some or all of the specific details described in the specification. In many cases, method steps described in this specification can be performed in different orders than that presented in this specification, or in parallel rather than sequentially.

The invention claimed is:

1. A method, comprising the steps of:
in a database. storing information relating to specific individual endoscopes, the endoscopes having respective image sensors of a type that has properties that vary from one sensor to another within manufacturing tolerances, the stored information including:
information recording properties of individual endoscopes' image sensors and/or the image sensors' behavior; and
physical location and/or ownership of individual endoscopes;
by an optical reader, reading 2D identification codes on packaging of the endoscopes, and updating the physical location and/or ownership database records to show current physical location and/or ownership based on the read 2D identification codes.
as respective endoscopes are put into use, by computer, reading a machine-readable serial number of the endoscope, and
based on the reading of the serial number, recording a reduction of an on-hand inventory level of a class of endoscopes of which the endoscope is a member;
based on the read serial number, recording a relation between the serial number's endoscope and a patent for a procedure in which the endoscope is to be used;
by computer, computing when the reduction of on-hand inventory of the class indicates a level that warrants reordering to replenish the on-hand inventory;
by computer, based on the computed warrant to reorder, placing an order with a supplier of endoscopes of the class to replenish on-hand inventory of endoscopes of the class;
based on the read serial number, obtaining from the database the recorded properties of the endoscope's image sensor, and computing normalized video based on the obtained properties and the video from the image sensor.

2. A method, comprising the steps of:
by computer, reading a machine-readable serial number of a medical device as the medical device is put into use, and based on the reading of the serial number, recording a reduction of an on-hand inventory level of a class of medical devices of which the medical device is a member;
by computer, computing when the reduction of on-hand inventory of the class indicates a level that warrants reordering to replenish the on-hand inventory; and
by computer, based on the computed warrant to reorder, placing an order with a supplier of medical devices of the class to replenish on-hand inventory of medical devices of the class.

3. The method of claim 2:
connecting a one of the medical devices to a computer processor, the processor programmed to obtain properties of the connected medical device from a database, the database being designed to store information relating to specific individual medical devices, the database being accessed based on the read serial number.

4. The method of claim 3:
the medical device being an endoscope having an image sensor, the image sensor designed to gather photons to produce video, the image sensor being of a type that has properties that vary from one sensor to another within manufacturing tolerances;
and further comprising the steps of:
storing properties of the endoscope's image sensor and/or the image sensor's behavior in a database, the database storing properties specific to specific sensors; and
the image processor programmed to obtain the properties of the one endoscope's image sensor from the database, and to compute normalized video based on the obtained properties and the video from the image sensor.

5. The method of claim 3:
the database being further designed to store physical location and/or ownership of individual medical devices;
and further comprising the steps of:
by an optical reader, reading identification codes on packaging of the medical devices; and
updating the physical location and/or ownership database records to show current physical location and/or ownership based on the read identification codes.

6. The method of claim 5:
the medical device being an endoscope having an image sensor, the image sensor designed to gather photons to produce video, the image sensor being of a type that has properties that vary from one sensor to another within manufacturing tolerances;
and further comprising the steps of:
storing properties of the endoscope's image sensor and/or the image sensor's behavior in a database, the database storing properties specific to specific sensors; and
the image processor programmed to obtain the properties of the one endoscope's image sensor from the database, and to compute normalized video based on the obtained properties and the video from the image sensor.

7. The method of claim 3, wherein:
the identification code is a machine-readable 2D optical code.

8. The method of claim 3, wherein:
the identification code is stored in machine-readable electronic memory.

9. The method of claim 3, wherein:
the database stores information relating to a device model of a class of medical devices manufactured to be interchangeable, the database designed to index to the device model information based on the read identification code of an individual medical device.

10. A computer system, comprising:
one or more processors; and
a machine readable, non-transitory memory having stored therein instructions programmed to cause the processor(s) to:
read a machine-readable serial number of a medical device as the medical device is put into use, and based on the reading of the serial number, to record a reduction of an on-hand inventory level of a class of medical devices of which the medical device is a member;
compute when the reduction of on-hand inventory of the class indicates a level that warrants reordering to replenish the on-hand inventory; and
based on the computed warrant to reorder, place an order with a supplier of medical devices of the class to replenish on-hand inventory of medical devices of the class.

11. The computer system of claim 10:
the medical device being an endoscope having an image sensor, the image sensor designed to gather photons to produce video, the image sensor being of a type that has properties that vary from one sensor to another within manufacturing tolerances;
the instructions being further programmed to cause the processor(s) to:
connect a one of the medical devices to a computer processor, the processor programmed to obtain properties of the connected medical device from a database, the database being designed to store information relating to specific individual medical devices, the database being accessed based on the read serial number;
store properties of the endoscope's image sensor and/or the image sensor's behavior in a database, the database storing properties specific to specific sensors; and
obtain the properties of the one endoscope's image sensor from the database, and to compute normalized video based on the obtained properties and the video from the image sensor.

12. The computer system of claim 10:
the database being further designed to store physical location and/or ownership of individual medical devices;
the instructions being further programmed to cause the processor(s) to:
direct an optical reader to read identification codes on packaging of the medical devices and connect a one of the medical devices to a computer processor, the processor programmed to obtain properties of the connected medical device from a database, the database being designed to store information relating to specific individual medical devices, the database being accessed based on the read serial number.
update the physical location and/or ownership database records to show current physical location and/or ownership based on the read identification codes.

13. An endoscope, comprising:
an image sensor designed to gather photons to produce video, the image sensor being of a type that has properties that vary from one sensor to another within manufacturing tolerances;
a connector to connect the endoscope to a computer image processor, the image processor programmed to:
obtain data from a database designed to store properties of the endoscope's image sensor and/or the image sensor's behavior, the database storing properties specific to specific sensors or a specific class of sensors;
the obtained data to describe the image sensor's properties from the database, and
compute normalized video based on the obtained properties and the video from the image sensor.

14. The endoscope of claim 13, further designed to connect to a computer processor programmed to:
read a machine-readable serial number of a endoscope as the endoscope is put into use, and based on the reading of the serial number, record a reduction of an on-hand inventory level of a class of endoscopes of which the endoscope is a member;
compute when the reduction of on-hand inventory of the class indicates a level that warrants reordering to replenish the on-hand inventory; and
based on the computed warrant to reorder, to place an order with a supplier of endoscopes of the class to replenish on-hand inventory of endoscopes of the class.

15. The endoscope of claim 13:
the database being further designed to store information relating to specific individual medical devices, including physical location and/or ownership;
by an optical reader, reading 2D identification codes on packaging of the medical devices; and
updating the physical location and/or ownership database records to show current physical location and/or ownership based on the read 2D identification codes.

16. The endoscope of claim 13, the database designed to store data describing a color spectrum capability of the image sensor.

17. The endoscope of claim 13, the database designed to store data describing an image plane resolution of the image sensor.

18. The endoscope of claim 13:
obtain data from a database designed to store calibration properties of the endoscope's image sensor specific to the specific image sensor of the specific endoscope;
the image processor being programmed to compute normalized video based on the obtained calibration properties and the video from the image sensor.

19. The endoscope of claim 18, wherein the calibration properties describe a white balance of the image sensor.

20. The endoscope of claim 18, wherein the calibration properties describe a color correction gamma curve of the image sensor.

21. The endoscope of claim 18, wherein the calibration properties describe a distortion correction of the image sensor.

22. A method comprising the steps of:
at a computer processor programmed to receive image date from an image sensor of an endoscope, obtaining data from a database designed to store properties of endoscopes' image sensors and/or the image sensors' behavior, the image sensor being of a type that has properties that vary from one sensor to another, the database storing properties specific to specific sensors or a specific class of sensors;
at the computer, compute normalized video based on the obtained properties and the video from the image sensor.

23. The method of claim 22, further comprising the steps of:
at a computer, reading a machine-readable serial number of a endoscope as the endoscope is put into use, and based on the reading of the serial number, record a reduction of an on-hand inventory level of a class of endoscopes of which the endoscope is a member;
computing when the reduction of on-hand inventory of the class indicates a level that warrants reordering to replenish the on-hand inventory; and
by computer, based on the computed warrant to reorder, placing an order with a supplier of endoscopes of the class to replenish on-hand inventory of endoscopes of the class.

24. The method of claim 22:
the database being further designed to store information relating to specific individual medical devices, including physical location and/or ownership;
by an optical reader, reading 2D identification codes on packaging of the medical devices; and
updating the physical location and/or ownership database records to show current physical location and/or ownership based on the read 2D identification codes.

25. The method of claim 22, further comprising the steps of:
obtain data from a database designed to store calibration properties of the endoscope's image sensor specific to the specific image sensor of the specific endoscope;
the image processor being programmed to compute normalized video based on the obtained calibration properties and the video from the image sensor.

26. The method of claim 25, wherein the calibration properties describe a white balance of the image sensor.

27. A method, comprising the steps of:
in a database. storing information relating to specific individual medical devices, including physical location and/or ownership;
by an optical reader, reading 2D identification codes on packaging of the medical devices; and
updating the physical location and/or ownership database records to show current physical location and/or ownership based on the read 2D identification codes.

28. The method of claim 27, further comprising the steps of:
by computer, reading a machine-readable serial number of a medical device as the medical device is put into use, and based on the reading of the serial number, recording a reduction of an on-hand inventory level of a class of medical devices of which the medical device is a member;
by computer, computing when the reduction of on-hand inventory of the class indicates a level that warrants reordering to replenish the on-hand inventory; and
by computer, based on the computed warrant to reorder, placing an order with a supplier of medical devices of the class to replenish on-hand inventory of medical devices of the class.

29. The method of claim 27:
the medical devices being endoscopes having respective image sensors, the image sensors designed to gather photons to produce video, the image sensors being of a type that has properties that vary from one sensor to another within manufacturing tolerances;
and further comprising the steps of:
storing properties of the endoscope's image sensor and/or the image sensor's behavior in a database, the database storing properties specific to specific sensors; and
connecting a one of the endoscopes to a computer image processor, the image processor programmed to obtain the properties of the one endoscope's image sensor from the database, and to compute normalized video based on the obtained properties and the video from the image sensor.

30. The method of claim 27, further comprising the step of:
for medical devices between the time of a sale and the time of delivery, computing a prediction of delivery date based on the read 2D identification codes.

31. The method of claim 27, further comprising the step of:
in a database. storing information relating to specific individual medical devices, including physical location;
by an optical reader, reading 2D identification codes on packaging of the medical devices; and
updating the physical location and/or ownership database records to show current physical location based on the read 2D identification codes.

32. The method of claim 27, further comprising the step of:
in a database. storing information relating to specific individual medical devices, including ownership;
by an optical reader, reading 2D identification codes on packaging of the medical devices; and
updating the physical location and/or ownership database records to show current ownership based on the read 2D identification codes.

33. A computer system, comprising:
one or more processors; and
a machine readable, non-transitory memory having stored therein instructions programmed to cause the processor(s) to:
in a database. storing information relating to specific individual medical devices, including physical location and/or ownership;
by an optical reader, reading 2D identification codes on packaging of the medical devices; and
updating the physical location and/or ownership database records to show current physical location and/or ownership based on the read 2D identification codes.

34. The computer system of claim 33, the instructions being further programmed to cause the processor(s) to:
read a machine-readable serial number of a medical device as the medical device is put into use, and based on the reading of the serial number, recording a reduction of an on-hand inventory level of a class of medical devices of which the medical device is a member;
compute when the reduction of on-hand inventory of the class indicates a level that warrants reordering to replenish the on-hand inventory; and
based on the computed warrant to reorder, place an order with a supplier of medical devices of the class to replenish on-hand inventory of medical devices of the class.

35. The computer system of claim 33:
the medical devices being endoscopes having respective image sensors, the image sensors designed to gather photons to produce video, the image sensors being of a type that has properties that vary from one sensor to another within manufacturing tolerances;
the instructions being further programmed to cause the processor(s) to:
store properties of the endoscope's image sensor and/or the image sensor's behavior in a database, the database storing properties specific to specific sensors; and
connect a one of the endoscopes to a computer image processor, the image processor programmed to obtain the properties of the one endoscope's image sensor from the database, and to compute normalized video based on the obtained properties and the video from the image sensor.
